(19) United States
(12) Patent Application Publication
(10) Pub. No.: US 2024/0376448 A1
(43) Pub. Date: Nov. 14, 2024

(54) ENGINEERED RNA POLYMERASE VARIANTS

(71) Applicant: Codexis, Inc., Redwood City, CA (US)

(72) Inventors: Jacob Hansen Artz, Black Hawk, CO (US); Martha Camacho Hernandez, Houston, TX (US); Jason Fell, Martinez, CA (US); Mathew G. Miller, San Carlos, CA (US); Jovana Nazor, Milpitas, CA (US); James D. Nguyen, Winnetka, CA (US); Anand Patel, San Ramon, CA (US); Zhe Rui, Newark, CA (US); Jenna Tawil Abyad, River Vale, NJ (US); Hugo Juhn, Bronx, NY (US); Jason Arnold Lotvin, West Nyack, NY (US); Qi Liu, Scarsdale, NY (US); Joanna Sztuba-Solinska, Orangeburg, NY (US)

(21) Appl. No.: 18/661,523

(22) Filed: May 10, 2024

Related U.S. Application Data
(60) Provisional application No. 63/502,017, filed on May 12, 2023.

(51) Int. Cl. C12N 9/12; C12N 15/63

(52) U.S. Cl. CPC C12N 9/1247; C12N 15/63; C12Y 207/07006

(57) ABSTRACT

The present disclosure relates to engineered RNA polymerase polypeptides and compositions thereof, as well as polynucleotides encoding the engineered RNA polymerase polypeptides. The present disclosure also provides methods of using the engineered RNA polymerase polypeptides or compositions thereof for producing RNA.

### CROSS-REFERENCE TO RELATED APPLICATIONS

This application claims the benefit of U.S. Provisional Application No. 63/502,017, filed May 12, 2023, which is incorporated by reference herein.

### REFERENCE TO SEQUENCE LISTING, TABLE OR COMPUTER PROGRAM

The Sequence Listing concurrently submitted herewith as file name CX9-243US1_ST26.xml, created on May 10, 2024, with a file size of 2,511,974 bytes, is part of the specification and is incorporated by reference herein.

### TECHNICAL FIELD

The present disclosure relates to engineered RNA polymerase variants, compositions thereof, and methods of using the engineered RNA polymerase variants.

### BACKGROUND

RNA polymerases transcribe a DNA, and in some instances RNA, into RNA transcripts. These enzymes represent the primary machinery that drives transcription. RNA polymerases have been isolated and purified sufficiently that they are useful for producing RNA in vitro. In vitro transcription allows for polynucleotide template directed synthesis of RNA molecules of any sequence, ranging in size from short oligonucleotides to several kilobases. Typically, in vitro transcription involves engineering of a template that includes a promoter sequence upstream of the sequence of interest followed by transcription using the corresponding RNA polymerase. RNA transcripts can be further modified by, among others, capping, splicing, and/or addition of a poly-A tail. Some modifications, such as capping and addition of a poly-A tail, can occur as part of the transcription reaction, for example, by co-transcriptional capping by the RNA polymerase and by inclusion of appropriate poly-dT sequences in the polynucleotide template. In other instances, the capping and addition of the poly-A tail can be done post-transcriptionally, for example by use of an RNA capping enzyme (e.g., Faustovirus or Vaccinia virus capping enzyme) and poly-A tailing with a poly(A) polymerase (e.g., E. coli. poly(A) polymerase). In vitro generated transcripts are used in analytical techniques (e.g., hybridization analysis), structural studies (e.g., NMR and X ray crystallography), in biochemical and genetic studies (e.g., as antisense reagents), as functional molecules (e.g., ribozymes and aptamers), and as therapeutic agents.

RNA has become the focus of therapeutic applications, including, among others mRNA based vaccines, cancer immunotherapeutic, genome engineering (e.g., CRISPR), and enzyme replacement supplementation therapies. Important to such applications is the availability of stable RNA polymerases that can produce RNA at high yield and with high fidelity, while having minimal amount of undesirable products, including, among others, incomplete transcripts, and double stranded RNA.

### SUMMARY

The present disclosure provides engineered RNA polymerase polypeptides and compositions thereof, as well as polynucleotides encoding the engineered RNA polymerase polypeptides, where the engineered RNA polymerases are engineered to have improved properties, such as increased activity, increased thermostability, increased processivity, and increased yield or integrity of RNA product. The present disclosure also provides methods of using the engineered RNA polymerase polypeptides and compositions thereof for nucleic acid synthesis and other purposes.

In one aspect, the present disclosure provides engineered RNA polymerases, or a functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of SEQ ID NO: 2, 4, 32, 44, 156, 234, 238, 386, 394, 588, 604, 686, 692, 712, or 800, or to a reference sequence corresponding to SEQ ID NO: 2, 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 2, or to the reference sequence corresponding to SEQ ID NO: 2, wherein the amino acid sequence comprises one or more substitutions relative to the reference sequence corresponding to residues 8 to 890 of SEQ ID NO: 2, or relative to the reference sequence corresponding to SEQ ID NO: 2.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 2, or relative to the reference sequence corresponding to SEQ ID NO: 2.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to residues 8 to 890 of an even numbered SEQ ID NO. of SEQ ID NOs: 4-828, or to the sequence corresponding to an even numbered SEQ ID NO. of SEQ ID NOs: 4-828, wherein the amino acid sequence comprises one or more substitutions relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution at amino acid position 14, 15, 17, 19, 21, 32, 33, 38, 45, 52, 54, 55, 56, 56, 57, 60, 61, 62, 63, 67, 69, 72, 75, 78, 81, 82, 83, 84, 89, 93, 95, 100, 105, 111, 115, 122, 124, 127, 131, 133, 135, 137, 138, 139, 140, 141, 143, 150, 160, 161, 162, 164, 166, 168, 169, 170, 171, 172, 173, 179, 185, 187, 193, 196, 207, 212, 217, 218, 221, 227, 237, 239, 240, 245, 246, 250, 253, 254, 257, 261, 272, 274, 275, 311, 336, 340, 342, 351, 357, 367, 375, 379, 382, 388, 391, 392, 394, 397, 401, 402, 403, 404, 419, 424, 437, 440, 494, 510, 514, 517, 527, 529, 530, 531, 533, 536, 589, 594, 598, 607, 609, 615, 616, 617, 625, 629, 648, 650, 655, 659, 660, 663, 664, 666, 670, 672, 690, 693, 696, 718, 720, 721, 749, 759, 763, 773, 775, 779, 782, 782, 810, 854, 857, 867, 875, or 878, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution at amino acid position 32, 38, 56, 138, 150, 170, 171, 173, 246, 311, 336, 340, 357, 388, 394, 401, 404, 437, 514, 536, 609, 629, 659, 660, 664, 779, or 782, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set at amino acid position(s) 401/404, 336, 138, 401/403/404, 336/510, 388, 340, 401/402/404, or 351/357, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution as set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set of an engineered RNA polymerase set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising at least a substitution or substitution set as set forth in Tables 5.1, 6.1, 7.1, 8.1, 9.1, 10.1, 11.1, 12.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to residues 8 to 890 of an even numbered SEQ ID NO. of SEQ ID NOs: 4-828, or to the reference sequence corresponding to an even numbered SEQ ID NO. of SEQ ID NOs: 4-828.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of an even-number SEQ ID NO. of SEQ ID NOs: 4-828, or to a reference sequence corresponding to an even-numbered SEQ ID NO. of SEQ ID NOs: 4-828, wherein the amino acid sequence comprises one or more substitutions relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the amino acid sequence of the engineered RNA polymerase comprises at least a substitution at amino acid position 13, 14, 15, 17, 19, 21, 32, 33, 38, 45, 52, 54, 55, 56, 56, 57, 60, 61, 62, 63, 67, 69, 72, 75, 78, 81, 82, 83, 84, 89, 93, 95, 100, 105, 111, 115, 122, 124, 127, 131, 133, 135, 137, 138, 139, 140, 141, 143, 150, 160, 161, 162, 164, 166, 168, 169, 170, 171, 172, 173, 179, 185, 187, 193, 196, 207, 212, 217, 218, 221, 227, 237, 239, 240, 245, 246, 250, 253, 254, 257, 261, 272, 274, 275, 311, 336, 340, 342, 351, 357, 367, 375, 379, 382, 388, 391, 392, 394, 397, 401, 402, 403, 404, 419, 424, 437, 440, 494, 510, 514, 517, 527, 529, 530, 531, 533, 536, 589, 594, 598, 607, 609, 615, 616, 617, 625, 629, 648, 650, 655, 659, 660, 663, 664, 666, 670, 672, 690, 693, 696, 718, 720, 721, 749, 759, 763, 773, 775, 779, 782, 782, 810, 854, 857, 867, 875, or 878, or combinations thereof, or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the amino acid sequence of the engineered RNA polymerase comprises at least a substitution at amino acid position 32, 38, 56, 138, 150, 170, 171, 173, 246, 311, 336, 340, 357, 388, 394, 401, 404, 437, 514, 536, 609, 629, 659, 660, 664, 779, or 782, or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, or to the reference sequence corresponding to SEQ ID NO: 4, wherein the amino acid sequence comprises one or more substitutions relative to the reference sequence corresponding to residues 8 to 890 of SEQ ID NO: 4, or relative to the reference sequence corresponding to SEQ ID NO: 4.

In some embodiments, the amino acid sequence of the engineered RNA polymerase comprises at least a substitution set at amino acid positions 311/388/404/440, 138/340, 138/311/336/340/388, 311/388/440, 138/311/340/388/404/440, 138/336/342, or 351/388, wherein the amino acid positions are relative to the reference sequence corresponding to residues 8 to 890 of SEQ ID NO: 4, or relative to the reference sequence corresponding to SEQ ID NO: 4.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 32, or to the reference sequence corresponding to SEQ ID NO: 32, wherein the amino acid sequence comprises one or more substitutions relative to the reference sequence corresponding to residues 8 to 890 of SEQ ID NO: 32, or relative to the reference sequence corresponding to SEQ ID NO: 32.

In some embodiments, the amino acid sequence of the engineered RNA polymerase comprises at least a substitution or substitution set at amino acid position(s) 514, 38/514, 38/394/514, 38/394/514/664, 394/514/875, 394/514, 38/124/394/514, 171/878, 170, 173, 172, 170/629, 171, 168, 166, 166/272/274/275, 169, 207, 168/217/218, 171/530, 382, 773, 670, 379, 392, 419, 57, 164, 810, 659, 54, 172/173/514/659/660, 170/171/172/514/659, 173/514/659/660, 173/514/659/810, 170/340/514/659/660/810, 170/171/173/336/340/514/659/660, 57/171/173/514/659/660/810, 170/173/514/659/660/810, 57/171/172/336/340/514/659, 173/514, 171/172/173/514/659/660, 57/170/171/173/514/659/660/810, 57/173/514, 172/173/514/857, 57/173/340/514, 170/171/173/336/514, 171/514, 57/170/173/336/514, 170/336, 173/514/659, 57/514, or 173/810, wherein the amino acid positions are relative to the reference sequence corresponding to residues 8 to 890 of SEQ ID NO: 32, or relative to the reference sequence corresponding to SEQ ID NO: 32.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 44, or to the reference sequence corresponding to SEQ ID NO: 44, wherein the amino acid sequence comprises one or more substitutions relative to the reference sequence corresponding to residues 8 to 890 of SEQ ID NO: 44, or relative to the reference sequence corresponding to SEQ ID NO: 44.

In some embodiments, the amino acid sequence of the engineered RNA polymerase comprises at least a substitution set at amino acid position(s) 336, 143/336/340/782 or 143/336/340/357/514, wherein the amino acid positions are relative to the reference sequence corresponding to residues 8 to 890 of SEQ ID NO: 44, or relative to the reference sequence corresponding to SEQ ID NO: 44.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 156, or to a reference sequence corresponding to SEQ ID NO: 156, wherein the amino acid sequence comprises one or more substitutions relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerase comprises at least a substitution or substitution set at amino acid position(s) 207, 419, 207/336/340, 166/169, 207/670, 166/336/340, 207/419/670, 207/336/340/382/419/670, 207/382, 670, 336/670, 340/670, 382, 127/141/207/336/382/670, 207/336/382/419/670, 419/670, 166/168/207/336/340/419/670, 166/340, or 207/336/340/670, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerase comprises at least a substitution or substitution set at amino acid position(s) 38/437/779, 38/437, 38, 38/207/437/779, 517/779, 38/437/527/664, 38/207/379/437, 664/670/779, 38/779, 38/437/517, 437/779, 38/437/607, 38/379/779, or 517/670/779, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerase comprises at least a substitution set at amino acid positions 357/394/664 or 138/311/336/340/357/388/394/404/664, wherein the amino acid positions are relative to the reference sequence corresponding to residues 8 to 890 of SEQ ID NO: 156, or relative to the reference sequence corresponding to SEQ ID NO: 156.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 234, or to the reference sequence corresponding to SEQ ID NO: 234, wherein the amino acid sequence comprises one or more substitutions relative to the reference sequence corresponding to residues 8 to 890 of SEQ ID NO: 234, or relative to the reference sequence corresponding to SEQ ID NO: 234.

In some embodiments, the amino acid sequence of the engineered RNA polymerase comprises at least a substitution or substitution set at amino acid position(s) 720, 196, 609, 246, 185, 594, 212, 494, 375, 721, 72, 867, 160, 718, 187, 170/173, 615, 137/138, 854, 131, 38/185/187/437, 185/187, 437/609, 38/609, 38/185/187/437/609, 437/609/720, 185/187/609, 185/187/437, 437/721, 609/720, 185/187/246, or 185/187/437/609/720, wherein the amino acid positions are relative to the reference sequence corresponding to residues 8 to 890 of SEQ ID NO: 234, or relative to the reference sequence corresponding to SEQ ID NO: 234.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 386, or to the reference sequence corresponding to SEQ ID NO: 386, wherein the amino acid sequence comprises one or more substitutions relative to the reference sequence corresponding to residues 8 to 890 of SEQ ID NO: 386, or relative to the reference sequence corresponding to SEQ ID NO: 386.

In some embodiments, the amino acid sequence of the engineered RNA polymerase comprises at least a substitution or substitution set at amino acid position(s) 170/609, 38/185/187/609, 38/185/187, 38/170/173, 38/173/185/187/720, 170/185/187/609, 609, 170/185/187/609/721, 38/609, 38/170/246/609, 185/187/609/718, 38/185/187/609/718/720, 38/170/246/609/718, 720, 246/609, 38/185/187/246/720, 38/609/721, 38/721, 38/720/721, 38/170/609/718/720, 38/718, 38/185/187/609/718/721, 529/609, 38/609/718, 185/187/609/718/720, 185/187/609/718/721, 38/170/718/720/721, 170/185/187/718/720, 38/173/185/187/246/609/720, 609/718/720, 170/185/187/718/720/721, 38/170/173/246/609/718, 246/721, 38/718/720, or 38/609/718/720, wherein the amino acid positions are relative to the reference sequence corresponding to residues 8 to 890 of SEQ ID NO: 386, or relative to the reference sequence corresponding to SEQ ID NO: 386.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 238, or to the reference sequence corresponding to SEQ ID NO: 238, wherein the amino acid sequence comprises one or more substitutions relative to the reference sequence corresponding to residues 8 to 890 of SEQ ID NO: 238, or relative to the reference sequence corresponding to SEQ ID NO: 238.

In some embodiments, the amino acid sequence of the engineered RNA polymerase comprises at least a substitution or substitution set at amino acid position(s) 143, 105, 775/779, 135, 100, 246, 779/782, 749, 237, 162, 83, 218, 227, 115, 274, 81, 122, 61, 32, 139, 75, 239, 257, 240, 69/161, 253, 14, 221, 84, 133/150, 60, 275, 89, 15, 254, 625, 14/67, 21, 93, 250, 217, 19, 95, 759, 62, or 140, wherein the amino acid positions are relative to the reference sequence corresponding to residues 8 to 890 of SEQ ID NO: 238, or relative to the reference sequence corresponding to SEQ ID NO: 238.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 394, or to the reference sequence corresponding to SEQ ID NO: 394, wherein the amino acid sequence comprises one or more substitutions relative to the reference sequence corresponding to residues 8 to 890 of SEQ ID NO: 394, or relative to the reference sequence corresponding to SEQ ID NO: 394.

In some embodiments, the amino acid sequence of the engineered RNA polymerase comprises at least a substitution or substitution set at amino acid position(s) 609/779, 246/609, 246, 609/775/779, 105, 143/609/775, 105/135/609/775, or 111/609, 609, wherein the amino acid positions are relative to the reference sequence corresponding to residues 8 to 890 of SEQ ID NO: 394, or relative to the reference sequence corresponding to SEQ ID NO: 394.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 588, or to the reference sequence corresponding to SEQ ID NO: 588, wherein the amino acid sequence comprises one or more substitutions relative to the reference sequence corresponding to residues 8 to 890 of SEQ ID NO: 588, or relative to the reference sequence corresponding to SEQ ID NO: 588.

In some embodiments, the amino acid sequence of the engineered RNA polymerase comprises at least a substitution or substitution set at amino acid position(s) 32, 531/533, 115, 83, 32/60/61, 55, 589, 357, 617, 172, 598, 56/193, 56, 536, 52, 17, 261, 78, 629, 63, 82, 207, 666, 150, 45, 135, 13, 33, or 245, wherein the amino acid positions are relative to the reference sequence corresponding to residues 8 to 890 of SEQ ID NO: 588, or relative to the reference sequence corresponding to SEQ ID NO: 588.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 604, or to the reference sequence corresponding to SEQ ID NO: 604, wherein the amino acid sequence comprises one or more substitutions relative to the reference sequence corresponding to residues 8 to 890 of SEQ ID NO: 604, or relative to the reference sequence corresponding to SEQ ID NO: 604.

In some embodiments, the amino acid sequence of the engineered RNA polymerase comprises at least a substitution or substitution set at amino acid position(s) 207/245/629, 17/56/150/598, 56/629, 536, 185, 56/150, 17/56/150/207/245/598/617/629, 56/150/536/598, 56/536, 185/666, 150, or 143/150/246/782, wherein the amino acid positions are relative to the reference sequence corresponding to residues 8 to 890 of SEQ ID NO: 604, or relative to the reference sequence corresponding to SEQ ID NO: 604.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 686, or to the reference sequence corresponding to SEQ ID NO: 686, wherein the amino acid sequence comprises one or more substitutions relative to the reference sequence corresponding to residues 8 to 890 of SEQ ID NO: 686, or relative to the reference sequence corresponding to SEQ ID NO: 686.

In some embodiments, the amino acid sequence of the engineered RNA polymerase comprises at least a substitution or substitution set at amino acid position(s) 143, 246/424/648/782, 246/659/782, 56/150/629/660/782, 721/782, 782, 196/659/782, or 720/721/782, wherein the amino acid positions are relative to the reference sequence corresponding to residues 8 to 890 of SEQ ID NO: 686, or relative to the reference sequence corresponding to SEQ ID NO: 686.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 692, or to the reference sequence corresponding to SEQ ID NO: 692, wherein the amino acid sequence comprises one or more substitutions relative to the reference sequence corresponding to residues 8 to 890 of SEQ ID NO: 692, or relative to the reference sequence corresponding to SEQ ID NO: 692.

In some embodiments, the amino acid sequence of the engineered RNA polymerase comprises at least a substitution or substitution set at amino acid position(s) 143/659/782, 166/171/246/311, 143/721, 782, or 143/171/659/782, wherein the amino acid positions are relative to the reference sequence corresponding to residues 8 to 890 of SEQ ID NO: 692, or relative to the reference sequence corresponding to SEQ ID NO: 692.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 712, or to the reference sequence corresponding to SEQ ID NO: 712, wherein the amino acid sequence comprises one or more substitutions relative to the reference sequence corresponding to residues 8 to 890 of SEQ ID NO: 712, or relative to the reference sequence corresponding to SEQ ID NO: 712.

In some embodiments, the amino acid sequence of the engineered RNA polymerase comprises at least a substitution or substitution set at amino acid position(s) 367, 382, 670, 672, 357, 388, 693, 397, 375, 137/382/782, 655, 137/382/392, 391, 137/655/721/763, 696, 663, 137/382, 137/382/392/650/655/782, 137/382/629/660/672, 616, 690, 179, 137, 357/382/670, 388/670/672, 357/375, 357/672, 629, or 655/670, wherein the amino acid positions are relative to the reference sequence corresponding to residues 8 to 890 of SEQ ID NO: 712, or relative to the reference sequence corresponding to SEQ ID NO: 712.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 800, or to the reference sequence corresponding to SEQ ID NO: 800, wherein the amino acid sequence comprises one or more substitutions relative to the reference sequence corresponding to residues 8 to 890 of SEQ ID NO: 800, or relative to the reference sequence corresponding to SEQ ID NO: 800.

In some embodiments, the amino acid sequence of the engineered RNA polymerase comprises at least a substitution or substitution set at amino acid position(s) 137, 56/311/382, 56/137/143/721, 137/392, 56/137/392, 56/137/143/404/720, 56/143/246, 56/392, 392/404, 392, 56/137, or 56/196/392, wherein the amino acid positions are relative to the reference sequence corresponding to residues 8 to 890 of SEQ ID NO: 800, or relative to the reference sequence corresponding to SEQ ID NO: 800.

In some embodiments, the amino acid sequence of the engineered RNA polymerase comprises at least a substitution as set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the amino acid sequence of the engineered RNA polymerase comprises at least a substitution or substitution set of an engineered RNA polymerase set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set as set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the engineered RNA polymerase comprises an amino acid sequence comprising residues 8 to 890 of an even numbered SEQ ID NO. of SEQ ID NOs: 4-828, or comprises an even numbered SEQ ID NO. of SEQ ID NOs: 4-828, optionally wherein the amino acid sequence has 1, 2, 3, 4, 5, 6, 7, 8, 9, or up to 10 substitutions.

In some embodiments, the engineered RNA polymerase comprises an amino acid sequence comprising residues 8 to 890 of SEQ ID NO: 4, 32, 44, 156, 234, 238, 386, 394, 588, 604, 686, 692, 712, or 800, or comprising SEQ ID NO: 4, 32, 44, 156, 234, 238, 386, 394, 588, 604, 686, 692, 712, or 800, optionally wherein the amino acid sequence has 1, 2, 3, 4, 5, 6, 7, 8, 9, or up to 10 substitutions.

In some embodiments, the engineered RNA polymerase is characterized by at least one improved property as compared to a reference RNA polymerase. In some embodiments, the improved property is selected from i) increased activity, ii) increased thermostability, iii) increased processivity, or iv) increased RNA product integrity, or any combination of i), ii), iii) and iv), as compared to a reference RNA polymerase, wherein the reference RNA polymerase has the sequence corresponding to residues 8 to 890 of SEQ ID NO: 2, 4, 32, 44, 156, 234, 238, 386, 394, 588, 604, 686, 692, 712, or 800, or the sequence corresponding to SEQ ID NO: 2, 4, 32, 44, 156, 234, 238, 386, 394, 588, 604, 686, 692, 712, or 800. In some embodiments, the reference RNA polymerase has the sequence corresponding to residues 8 to 890 of SEQ ID NO: 2, or the sequence corresponding to SEQ ID NO: 2.

In some further embodiments, the engineered RNA polymerase is purified. In some embodiments, the engineered RNA polymerase is provided in solution, as a lyophilizate, or is immobilized on a substrate or support medium, such as surfaces of solid substrates or membranes or particles.

In another aspect, the present disclosure provides recombinant polynucleotides encoding the engineered RNA polymerases disclosed herein.

In some embodiments, the recombinant polynucleotide comprises a polynucleotide sequence having at least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 21 to 2670 of an odd-numbered SEQ ID NO. of SEQ ID NOs: 3-827, or to an odd-numbered SEQ ID NO. of SEQ ID NOs: 3-827, wherein the recombinant polynucleotide encodes an RNA polymerase.

In some embodiments, the recombinant polynucleotide comprises a polynucleotide sequence having at least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 21 to 2670 of SEQ ID NO: 3, 31, 43, 155, 233, 237, 381, 393, 587, 603, 685, 691, 711, or 799, or a reference polynucleotide sequence corresponding to SEQ ID NO: 3, 31, 43, 155, 233, 237, 381, 393, 587, 603, 685, 691, 711, or 799, wherein the recombinant polynucleotide encodes an RNA polymerase.

In some embodiments, the polynucleotide sequence of the recombinant polynucleotide encoding an engineered RNA polymerase is codon optimized. In some embodiments, the polynucleotide sequence is codon optimized for expression in a bacterial cell, fungal cell, insect cell, or mammalian cell.

In some embodiments, the recombinant polynucleotide comprises a polynucleotide sequence comprising nucleotide residues 21 to 2670 of an odd-numbered SEQ ID NO. of SEQ ID NOs: 3-827, or comprising an odd numbered SEQ ID NO. of SEQ ID NOs: 3-827.

In a further aspect, the present disclosure provides expression vectors comprising at least one recombinant polynucleotide encoding an engineered RNA polymerase described herein. In some embodiments, the recombinant polynucleotide in the expression vector is operably linked to a control sequence. In some embodiments, the control sequence comprises a promoter, particularly a heterologous promoter.

In another aspect, the present disclosure also provides a host cell transformed with at least one expression vector provided herein. In some embodiments, the host cell is a prokaryotic cell or a eukaryotic cell. In some embodiments, the host cell is a bacterial cell, fungal cell, insect cell, or mammalian cell. In some preferred embodiments, the host cell is a bacterial cell, such as E. coli. or B. subtilis.

In a further aspect, the present disclosure provides a method of producing an engineered RNA polymerase polypeptide, the method comprising culturing a host cell described herein under suitable culture conditions such that at least one engineered RNA polymerase is produced. In some embodiments, the method further comprises recovering or isolating the expressed engineered RNA polymerase from the culture and/or host cells. In some embodiments, the method further comprises purifying the engineered RNA polymerase.

In another aspect, the present disclosure provides a composition comprising at least one engineered RNA polymerase disclosed herein. In some embodiments, the composition further comprises at least a buffer. In some embodiments, the composition further comprises a reducing agent, such as dithiothreitol or mercaptoethanol. In some embodiments, the composition further comprises one or more nucleotide triphosphate (NTP) substrates, particularly rNTP substrates. In some embodiments, the composition further comprises a cap analog. In some embodiments, the composition further comprises a target DNA template. In some embodiments, the target DNA template comprises a promoter recognized by the engineered RNA polymerase. In some embodiments, the composition further comprises a pyrophosphatase and/or an RNase inhibitor. In some embodiments, the composition further comprises an additive, such as a molecular crowding agent.

In a further aspect, the present disclosure provides a method of producing RNA, the method comprising contacting a target DNA template with an engineered RNA polymerase described herein in presence of one or more nucleotide triphosphates under conditions suitable for transcription of the target DNA template. In some embodiments, the method further comprises providing a cap analog. In some embodiments, the method further comprises a pyrophosphatase and/or an RNase inhibitor in the reaction with the engineered RNA polymerase. In some embodiments, the suitable conditions comprise a temperature of about 25° C. to about 40° C. In some embodiments, the suitable conditions include an additive, such as a molecular crowding agent.

In a further aspect, the present disclosure also provides a kit comprising at least one engineered RNA polymerase disclosed herein. In some embodiments, the kit further comprises one or more of a buffer, one or more rNTPs, Mg⁺², a reducing agent, and a molecular crowding agent. In some embodiments, the kit further comprises a suitable DNA template.

### DETAILED DESCRIPTION

The present disclosure provides engineered RNA polymerase polypeptides and compositions thereof, where the engineered RNA polymerase displays one or more improved properties, including, among others, increased activity, increased stability, increased thermal stability, and increased product integrity. The present disclosure further provides recombinant polynucleotides encoding the engineered RNA polymerase polypeptides and methods of using the engineered RNA polymerase for producing RNA. In some embodiments, the expressed RNA encodes a polypeptide or is a non-coding RNA (ncRNA), such as shRNA, miRNA, or guide RNA.

### Abbreviations and Definitions

In reference to the present disclosure, the technical and scientific terms used in the descriptions herein will have the meanings commonly understood by one of ordinary skill in the art, unless specifically defined otherwise.

It is to be understood that this invention is not limited to the particular methodology, protocols, and reagents described, as these may vary, depending upon the context they are used by those of skill in the art. Accordingly, the terms defined immediately below are more fully described by reference to the application as a whole.

As used herein, the singular “a”, “an,” and “the” include the plural references, unless the context clearly indicates otherwise.

As used herein, the term “comprising” and its cognates are used in their inclusive sense (i.e., equivalent to the term “including” and its corresponding cognates).

It is also to be understood that where description of embodiments use the term “comprising” and its cognates, the embodiments can also be described using language “consisting essentially of” or “consisting of.”

Moreover, numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glycine (Gly or G), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (Cα). For example, whereas “Ala” designates alanine without specifying the configuration about the α carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“RNA polymerase” or “RNAP” refers to an enzyme that catalyzes the synthesis of RNA in the 5′ to 3′ direction using a polynucleotide as a template. In some embodiments, the RNA polymerase uses a DNA template and is, in some embodiments, referred to as a DNA-directed or DNA-dependent RNA polymerase. In some embodiments, the RNA polymerase uses an RNA template and is, in some embodiments, referred to as an RNA-directed or RNA-dependent RNA polymerase. In some embodiments, an RNA polymerase is capable of using DNA and RNA as a template for synthesis of the RNA. In some embodiments, the RNA product is referred to as a “RNA transcript.”

“Cap” as used herein refers to the nucleoside that is joined via its 5′ carbon to a triphosphate group that is, in turn joined to the 5′ carbon of the most 5′ nucleotide of an RNA transcript. In some embodiments, the nucleoside of the cap is a guanine (G). In some embodiments, the nitrogen at the 7-position of guanine in the cap is methylated and is denoted as m7G. In some embodiments, the cap is a dinucleotide cap, trinucleotide cap, or a tetranucleotide cap. The terms “capped RNA,” “5′ capped RNA,” and “capped mRNA” refer to RNA and mRNA, respectively that comprise the cap.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“Polynucleotide,” “nucleic acid,” or “oligonucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When polynucleotide, nucleic acid, or oligonucleotide sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid. The polynucleotide or nucleic acid may be single-stranded or double-stranded, or may include both single-stranded regions and double-stranded regions.

“Duplex” and “ds” refer to a double-stranded nucleic acid (e.g., DNA or RNA) molecule comprised of two single-stranded polynucleotides that are complementary in their sequence (A pairs to T or U, C pairs to G), arranged in an antiparallel 5′ to 3′ orientation, and held together by hydrogen bonds between the nucleobases (e.g., adenine [A], guanine [G], cytosine [C], thymine [T], uridine [U]).

“Engineered,” “recombinant,” “non-naturally occurring,” and “variant,” when used with reference to a cell, a polynucleotide or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally occurring” refer to the form found in nature. For example, a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein.

“Percent (%) sequence identity” refers to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et al., J. Mol. Biol., 1990, 215: 403-410; and Altschul et al., Nucleic Acids Res., 1977, 3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (see Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “a reference sequence corresponding to SEQ ID NO: 2, having a glycine (G) at the residue corresponding to X38” (or “a reference sequence corresponding to SEQ ID NO: 2, having an glycine (G) at the residue corresponding to position 38”) refers to a reference sequence in which the corresponding residue at position X38 in SEQ ID NO: 2 (e.g., an arginine, R), has been changed to glycine (G).

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to”, “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered RNA polymerase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. In some embodiments, the reference sequence is tagged (e.g., with a histidine tag).

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence.

“Amino acid difference” and “residue difference” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X38 as compared to SEQ ID NO: 2” (or a “residue difference at position 38 as compared to SEQ ID NO: 2”) refers to a difference of the amino acid residue at the polypeptide position corresponding to position 38 of SEQ ID NO: 2. Thus, if the reference polypeptide of SEQ ID NO: 2 has an arginine (R) at position 38, then a “residue difference at position X38 as compared to SEQ ID NO: 2” refers to an amino acid substitution of any residue other than arginine (R) at the position of the polypeptide corresponding to position 38 of SEQ ID NO: 2. In some instances herein, the specific amino acid residue difference at a position is indicated as “XnY” where “Xn” specified the corresponding residue and position of the reference polypeptide (as described above), and “Y” is the single letter identifier of the amino acid found in the engineered polypeptide (i.e., the different residue than in the reference polypeptide). In some instances (e.g., in the Tables in the Examples), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some embodiments, the amino acid difference, e.g., a substitution, is denoted by the abbreviation “nB,” without the identifier for the residue in the reference sequence. In some embodiments, the phrase “an amino acid residue nB” denotes the presence of the amino residue in the engineered polypeptide, which may or may not be a substitution in context of a reference sequence. In some embodiments, the “substitution” comprises the deletion of an amino acid, and can be denoted by “−” symbol.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X340E/X340W, X340E/W, X340E/W or 340E/W). The present disclosure includes engineered polypeptide sequences comprising one or more amino acid differences that include either/or both conservative and non-conservative amino acid substitutions, as well as insertions and deletions of amino acids in the sequence.

“Amino acid substitution set” and “substitution set” refers to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the variant RNA polymerase polypeptides listed in any of the Tables in the Examples. In these substitution sets, the individual substitutions are separated by a semicolon (e.g., V401R;Y404L) or slash (“/”; e.g., V401R/Y404L or 401R/404L).

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affect: (a) the structure of the peptide backbone in the area of the substitution (e.g., proline for glycine); (b) the charge or hydrophobicity; and/or (c) the bulk of the side chain. By way of example and not limitation, exemplary non-conservative substitutions include an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide while retaining biological activity and/or retaining the improved properties of an engineered RNA polymerase. Deletions can be directed to the internal portions and/or terminal portions of the polypeptide. In various embodiments, the deletion can comprise a continuous segment or can be discontinuous. Deletions are indicated by “−”, and may be present in substitution sets.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full length engineered RNA polymerase of the present disclosure) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally occurring environment or expression system (e.g., host cell or in vitro synthesis). The recombinant RNA polymerase polypeptides may be present within a cell, present in the cellular medium, or prepared in various forms, such as lysates or isolated preparations. As such, in some embodiments, the recombinant RNA polymerase polypeptides provided herein are isolated polypeptides.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure RNA polymerase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant RNA polymerase polypeptides are substantially pure polypeptide compositions.

“Improved property” refers to an engineered RNA polymerase polypeptide that exhibits an improvement in any RNA polymerase property as compared to a reference RNA polymerase polypeptide, such as a wild-type RNA polymerase polypeptide or another engineered RNA polymerase polypeptide. Improved properties include, but are not limited to, such properties as increased protein expression, increased activity, increased stability, increased thermostability, increased pH stability, increased chemical stability, improved solvent stability, increased solubility, and increased product integrity.

“Increased enzyme activity” and “enhanced enzyme activity” refer to an improved property of the engineered RNA polymerase polypeptides, which can be represented by an increase in specific activity as compared to the reference RNA polymerase polypeptide (e.g., wild-type RNA polymerase and/or another engineered RNA polymerase). Exemplary methods to determine enzyme activity are provided in the Examples. Improvements in enzyme activity can be from about 1.1 fold the enzyme activity of the corresponding wild-type or reference polypeptide, to about 1.5 fold, 2-fold, 5-fold, 10-fold, 20-fold, 25-fold, 50-fold, 75-fold, 100-fold, 150-fold, 200-fold or more enzyme activity than the naturally-occurring RNA polymerase or another engineered RNA polymerase from which the RNA polymerase polypeptides were derived.

“Processivity” in context of an RNA polymerase refers to the ability of the nucleic acid polymerase to catalyze the production of the total RNA product. In some embodiments, processivity is measured by the titer of the total RNA product, which is the concentration of the total RNA, for example, as measured by the methods described in the Examples. In some embodiments, increased “processivity” in context of nucleic acid polymerase refers to the increase in titer of full-length product compared with a reference polymerase.

“Integrity” in context of RNA product refers to the homogeneity of the RNA product from a transcription reaction, e.g., in vitro transcription reaction (IVT). In some embodiments, “integrity” is defined as the amount of the desired or target full-length RNA divided by the total amount of RNA produced, e.g., the area of the full length RNA electrophoretic peak divided by the area of the electropherogram trace through Fragment Analyzer, as described in the Examples. “Integrity score” is the reciprocal of one minus integrity:

$$Integrity score = \frac{1}{1 - integrity} = \frac{1}{1 - \frac{desired RNA peak area}{total RNA area}} .$$

An increased “integrity” or “integrity score” in context of RNA product refers to the increase in purity of full-length product compared with that from a reference transcription reaction e.g., IVT.

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the RNA polymerase polypeptides are codon optimized for optimal production from the host organism selected for expression.

“Control sequence” refers herein to include all components that are necessary or advantageous for the expression of a polynucleotide and/or polypeptide of the present disclosure. Each control sequence may be native or foreign to the nucleic acid sequence encoding the polypeptide. Such control sequences include, but are not limited to, leaders, polyadenylation sequences, propeptide sequences, promoter sequences, signal peptide sequences, initiation sequences, and transcription terminators. In some embodiments, at a minimum, the control sequences include a promoter, and transcriptional and translational stop signals. In some embodiments, the control sequences are provided with linkers for the purpose of introducing specific restriction sites facilitating ligation of the control sequences with the coding region of the nucleic acid sequence encoding a polypeptide.

“Operably linked” or “operatively linked” refers to a configuration in which a control sequence is appropriately placed (i.e., in a functional relationship) at a position relative to a polynucleotide of interest such that the control sequence directs or regulates the expression of the polynucleotide, and where relevant, expression of an encoded polypeptide of interest.

“Promoter” or “promoter sequence” refers to a nucleic acid sequence that is recognized by a host cell for expression of a polynucleotide of interest, such as a coding sequence. The promoter sequence contains transcriptional control sequences that mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell. In some embodiments, a “promoter” or “promoter sequence” refers to the nucleic acid sequence recognized by an engineered RNA polymerase.

“Suitable reaction conditions” or “suitable conditions” refers to those conditions (e.g., temperature, pH, buffers, salts, co-solvents, etc.) under which an RNA polymerase of the present disclosure is capable of synthesizing an RNA transcript. Exemplary “suitable conditions” are provided herein (see, e.g., the Examples).

“Culturing” refers to the growing of a population of cells, e.g., host cells, under suitable conditions using any suitable medium (e.g., liquid, gel, or solid).

“Vector” is a recombinant construct for introducing a polynucleotide of interest into a cell. In some embodiments, the vector is an expression vector that is operably linked to a suitable control sequence capable of effecting the expression in a suitable host of the polynucleotide and/or a polypeptide encoded in the polynucleotide. In some embodiments, an “expression vector” has a promoter sequence operably linked to the polynucleotide (e.g., transgene) to drive expression in a host cell, and in some embodiments, also comprises a transcription terminator sequence.

“Expression” includes any step involved in the production of the polypeptide including, but not limited to, transcription, post-transcriptional modification, translation, and post-translational modification. In some embodiments, the term also encompasses secretion of the polypeptide from a cell.

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Host cell” and “host strain” refer to suitable hosts for expression vectors comprising a polynucleotide provided herein (e.g., a polynucleotide sequences encoding at least one RNA polymerase variant). In some embodiments, the host cells are prokaryotic or eukaryotic cells that have been transformed or transfected with vectors constructed using recombinant DNA techniques as known in the art.

### Engineered RNA Polymerase Polypeptides

In one aspect, the present disclosure provides RNA polymerases engineered to have improved properties, including, among others, increased activity, increased stability, increased thermostability, and/or increased product integrity. In some embodiments, the engineered RNA polymerase variants find use in applications for preparing RNA and other molecular biological techniques.

In some embodiments, the engineered RNA polymerase, or a functional fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of SEQ ID NO: 2, 4, 32, 44, 156, 234, 238, 386, 394, 588, 604, 686, 692, 712, or 800, or to a reference sequence corresponding to SEQ ID NO: 2, 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 2, or to the reference sequence corresponding to SEQ ID NO: 2, wherein the amino acid sequence comprises one or more substitutions relative to the reference sequence corresponding to residues 8 to 890 of SEQ ID NO: 2, or relative to the reference sequence corresponding to SEQ ID NO: 2.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 2, or relative to the reference sequence corresponding to SEQ ID NO: 2.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 8 to 890 of an even numbered SEQ ID NO. of SEQ ID NOs: 4-828, or to a reference sequence corresponding to an even numbered SEQ ID NO. of SEQ ID NOs: 4-828, wherein the amino acid sequence comprises one or more substitutions relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution at amino acid position 13, 14, 15, 17, 19, 21, 32, 33, 38, 45, 52, 54, 55, 56, 56, 57, 60, 61, 62, 63, 67, 69, 72, 75, 78, 81, 82, 83, 84, 89, 93, 95, 100, 105, 111, 115, 122, 124, 127, 131, 133, 135, 137, 138, 139, 140, 141, 143, 150, 160, 161, 162, 164, 166, 168, 169, 170, 171, 172, 173, 179, 185, 187, 193, 196, 207, 212, 217, 218, 221, 227, 237, 239, 240, 245, 246, 250, 253, 254, 257, 261, 272, 274, 275, 311, 336, 340, 342, 351, 357, 367, 375, 379, 382, 388, 391, 392, 394, 397, 401, 402, 403, 404, 419, 424, 437, 440, 494, 510, 514, 517, 527, 529, 530, 531, 533, 536, 589, 594, 598, 607, 609, 615, 616, 617, 625, 629, 648, 650, 655, 659, 660, 663, 664, 666, 670, 672, 690, 693, 696, 718, 720, 721, 749, 759, 763, 773, 775, 779, 782, 782, 810, 854, 857, 867, 875, or 878, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or amino acid residue 13N, 14Q/Y, 15T, 17N, 19E, 21G, 32S/T/V, 33E, 38G, 45K, 52T, 54H, 55D/S/W, 56E/G/N/T/V, 57G, 60G/L/M/R, 61W/Y, 62T, 63S, 67R, 69S, 72R, 75S/Y, 78S, 81C/L, 82S, 83K, 84A, 89Q, 93Y, 95Y, 100R, 105R, 111K, 115L/V, 122I, 124F, 127G, 131T, 133C, 135G/K, 137G, 138R, 139R, 140R, 141A, 143N/Q/R/V, 150Q/Y, 160F/L, 161R, 162V, 164W, 166D/R/T/V, 168K/L/R/S/T/V/W, 169G/L, 170L/Q/R/S, 171E/F/L/Y, 172A/E/G/K/R/V, 173D/H/L/Y, 179N, 185I/L/P, 187G/H/L, 193G, 196A/C/G/S/V, 207S, 212R/Y, 217P/V, 218Q/T, 221C, 227V, 237L, 239L/R, 240M, 245C, 246K/R/V, 250R, 253Y, 254G/S, 257L/V, 261M, 272N, 274G/L, 275A/Y, 311G, 336L/M/S/V, 340E/W, L342Q, 351L, 357E/P, 367F/P/T, 375F/P, 379A/S, 382G/L/R, 388K/R, 391F/S, 392A/M/R, 394K, 397S, 401R, 402T, 403V, 404I/L/S/Y, 419R/S, 424A, 437P, 440K, 494G, 510V, 514I/S, 517R, 527E, 529R, 530N, 531Q, 533S, 536E/V, 589C, 594G, 598A/K/L/R/V, 607Y, 609C/E/F/P/W, 615A, 616A, 617R, 625C, 629D/K/R, 648F, 650M, 655M/R, 659R, 660H/W/Y, 663L, 664K/P, 666M, 670Q/R, 672G/P/R/S/T, 690S, 693R, 696L, 718F/N/S/V/Y, 720A/G/I/P/S, 721A/E/M/S/T/Y, 749R, 759M, 763S, 773A, 775K/R, 779R, 782R/V, 810K, 854T, 857V, 867Q, 875C, or 878M, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution I13N, A14Q/Y, K15T, D17N, S19E, I21G, A32S/T/V, D33E, R38G, A45K, E52T, G54H, E55D/S/W, A56E/G/N/T/V, R57G, K60G/L/M/R, M61W/Y, F62T, E63S, K67R, G69S, A72R, A75S/Y, K78S, 181C/L, T82S, T83K, L84A, I89Q, N93Y, W95Y, K100R, K105R, Q111K, E115L/V, A122I, I124F, K127G, A131T, L133C, S135G/K, D137G, N138R, T139R, T140R, V141A, A143N/Q/R/V, R150Q/Y, R160F/L, I161R, R162V, L164W, A166D/R/T/V, H168K/L/R/S/T/V/W, F169G/L, K170L/Q/R/S, K171E/F/L/Y, N172A/E/G/K/R/V, V173D/H/L/Y, K179N, Y185I/L/P, K187G/H/L, V193G, D196A/C/G/S/V, A207S, H212R/Y, 1217P/V, H218Q/T, V221C, L227V, H237L, Q239L/R, N240M, G245C, Q246K/R/V, T250R, L253Y, A254G/S, Y257L/V, I261M, S272N, M274G/L, F275A/Y, A311G, K336L/M/S/V, K340E/W, L342Q, W351L, R357E/P, L367F/P/T, D375F/P, E379A/S, T382G/L/R, A388K/R, V391F/S, Y392A/M/R, R394K, A397S, V401R, R402T, I403V, L404I/S/Y, K419R/S, P424A, S437P, N440K, E494G, A510V, L514I/S, C517R, G527E, Q529R, H530N, H531Q, L533S, N536E/V, L589C, I594G, D598A/K/L/R/V, E607Y, T609C/E/F/P/W, K615A, V616A, K617R, G625C, A629D/K/R, S648F, E650M, Q655M/R, E659R, D660H/W/Y, Q663L, W664K/P, I666M, K670Q/R, L672G/P/R/S/T, E690S, S693R, V696L, K718F/N/S/V/Y, K720A/G/I/P/S, K721A/E/M/S/T/Y, P749R, L759M, R763S, D773A, E775K/R, H779R, E782R/V, G810K, D854T, A857V, K867Q, G875C, or N878M, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution at amino acid position 32, 38, 56, 138, 150, 170, 171, 173, 246, 311, 336, 340, 357, 388, 394, 401, 404, 437, 514, 536, 609, 629, 659, 660, 664, 779, or 782, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or amino acid residue 32V, 38G, 56T, 138R/N, 150Y, 170Q, 171E, 173Y, 246K, 311A/G, 336K/S, 340K/W, 357E, 388A/K, 394K, 401R, 404L/S, 437P, 514S, 536V, 609E, 629A, 659R, 660H/W, 664P, 779R, or 782V, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution A32V, R38G, A56T, N138R/N, R150Y, K170Q, K171E, V173Y, Q246K, A311G, K336K/S, K340E/W, R357E, A388K/R, R394K, V401R, Y404L/S, S437P, L514S, N536V, T609E, R629A, E659R, D660H/W, W664P, H779R, or E782V, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set at amino acid position(s) 401/404, 138/311/336/340/388, 38/514, 170/171/173/336/340/514/659/660, 38/437/779, 357/394/664, 38, 138/311/336/340/357/388/394/404/664, 246/609, 32, 536, 56/150, 56/150/629/660/782, or 629, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set, or amino acid residue(s) 401R/404L, 138R/311G/336S/340W/388K, 38G/514S, 170Q/171E/173Y/336K/340K/514S/659R/660H, 38G/437P/779R, 357E/394K/664P, 38G, 138N/311A/336K/340K/357E/388A/394K/404S/664P, 246K/609E, 32V, 536V, 56T/150Y, 56T/150Y/629A/660W/782V, or 629K, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set V401R/Y404L, N138R/A311G/K336S/K340W/A388K, R38G/L514S, K170Q/K171E/V173Y/S336K/W340K/L514S/E659R/D660H, R38G/S437P/H779R, R357E/R394K/W664P, R38G, R138N/G311A/S336K/W340K/R357E/K388A/R394K/L404S/W664P, Q246K/T609E, A32V, N536V, A56T/R150Y, E56T/R150Y/R629A/H660W/E782V, or A629K, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set at amino acid position(s) 401/404, 336, 138, 401/403/404, 336/510, 388, 340, 401/402/404, or 351/357, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set, or amino acid residue(s) 401R/404L, 336S, 138R, 336V, 401R/403V/404S, 336M/510V, 336L, 388K, 340E, 401R/402T/404S, 351L/357E, or 401R/404I, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set V401R/Y404L, K336S, N138R, K336V, V401R/I403V/Y404S, K336M/A510V, K336L, A388K, K340E, V401R/R402T/Y404S, W351L/R357E, or V401R/Y404I, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 311/388/401/440, 138/340/401/404, 138/311/336/340/388/401/404, 311/388/401/404/440, 138/311/340/388/401/440, 138/336/342/401/404, or 351/388/401/404, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residues 311G/388K/401R/440K, 138R/340W/401R/404L, 138R/311G/336S/340W/388K/401R/404L, 311G/388K/401R/404L/440K, 138R/311G/340E/388K/401R/440K, 138R/336M/342Q/401R/404L, or 351L/388K/401R/404L, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 138/311/336/340/388/401/404/514, 38/138/311/336/340/388/401/404/514, 38/138/311/336/340/388/394/401/404/514, 38/138/311/336/340/388/394/401/404/514/664, 138/311/336/340/388/394/401/404/514/875, 138/311/336/340/388/394/401/404/514, 38/124/138/311/336/340/388/394/401/404/514, 138/171/311/336/340/388/401/404/878, 138/170/311/336/340/388/401/404, 138/173/311/336/340/388/401/404, 138/172/311/336/340/388/401/404, 138/170/311/336/340/388/401/404/629, 138/171/311/336/340/388/401/404, 138/168/311/336/340/388/401/404, 138/166/311/336/340/388/401/404, 138/166/272/274/275/311/336/340/388/401/404, 138/169/311/336/340/388/401/404, 138/207/311/336/340/388/401/404, 138/168/217/218/311/336/340/388/401/404, 138/171/311/336/340/388/401/404/530, 138/311/336/340/382/388/401/404, 138/311/336/340/388/401/404/773, 138/311/336/340/388/401/404/670, 138/311/336/340/379/388/401/404, 138/311/336/340/388/392/401/404, 138/311/336/340/388/401/404/419, 57/138/311/336/340/388/401/404, 138/164/311/336/340/388/401/404, 138/311/336/340/388/401/404/810, 138/311/336/340/388/401/404/659, 54/138/311/336/340/388/401/404, 138/172/173/311/336/340/388/401/404/514/659/660, 138/170/171/172/311/336/340/388/401/404/514/659, 138/173/311/336/340/388/401/404/514/659/660, 138/173/311/336/340/388/401/404/514/659/810, 138/170/311/336/388/401/404/514/659/660/810, 138/170/171/173/311/388/401/404/514/659/660, 57/138/171/173/311/336/340/388/401/404/514/659/660/810, 138/170/173/311/336/340/388/401/404/514/659/660/810, 57/138/171/172/311/388/401/404/514/659, 138/173/311/336/340/388/401/404/514, 138/171/172/173/311/336/340/388/401/404/514/659/660, 57/138/170/171/173/311/336/340/388/401/404/514/659/660/810, 57/138/173/311/336/340/388/401/404/514, 138/172/173/311/336/340/388/401/404/514/857, 57/138/173/311/336/388/401/404/514, 138/170/171/173/311/340/388/401/404/514, 138/171/311/336/340/388/401/404/514, 57/138/170/173/311/340/388/401/404/514, 138/170/311/340/388/401/404, 138/173/311/336/340/388/401/404/514/659, 57/138/311/336/340/388/401/404/514, or 138/173/311/336/340/388/401/404/810, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set, or amino acid residues 138R/311G/336S/340W/388K/401R/404L/514S, 38G/138R/311G/336S/340W/388K/401R/404L/514S, 38G/138R/311G/336S/340W/388K/394K/401R/404L/514S, 38G/138R/311G/336S/340W/388K/394K/401R/404L/514S/664P, 138R/311G/336S/340W/388K/394K/401R/404L/514S/875C, 138R/311G/336S/340W/388K/394K/401R/404L/514S, 38G/124F/138R/311G/336S/340W/388K/394K/401R/404L/514I, 138R/171E/311G/336S/340W/388K/401R/404L/878M, 138R/170Q/311G/336S/340W/388K/401R/404L, 138R/173Y/311G/336S/340W/388K/401R/404L, 138R/172V/311G/336S/340W/388K/401R/404L, 138R/173L/311G/336S/340W/388K/401R/404L, 138R/172E/311G/336S/340W/388K/401R/404L, 138R/170S/311G/336S/340W/388K/401R/404L/629D, 138R/171F/311G/336S/340W/388K/401R/404L, 138R/168V/311G/336S/340W/388K/401R/404L, 138R/172K/311G/336S/340W/388K/401R/404L, 138R/166R/311G/336S/340W/388K/401R/404L, 138R/168K/311G/336S/340W/388K/401R/404L, 138R/166V/272N/274L/275Y/311G/336S/340W/388K/401R/404L, 138R/170R/311G/336S/340W/388K/401R/404L, 138R/169L/311G/336S/340W/388K/401R/404L, 138R/207S/311G/336S/340W/388K/401R/404L, 138R/169G/311G/336S/340W/388K/401R/404L, 138R/168L/217P/218Q/311G/336S/340W/388K/401R/404L, 138R/168W/311G/336S/340W/388K/401R/404L, 138R/172R/311G/336S/340W/388K/401R/404L, 138R/166D/311G/336S/340W/388K/401R/404L, 138R/171L/311G/336S/340W/388K/401R/404L, 138R/171Y/311G/336S/340W/388K/401R/404L/530N, 138R/172A/311G/336S/340W/388K/401R/404L, 138R/173H/311G/336S/340W/388K/401R/404L, 138R/311G/336S/340W/382R/388K/401R/404L, 138R/311G/336S/340W/388K/401R/404L/773A, 138R/168R/311G/336S/340W/388K/401R/404L, 138R/311G/336S/340W/388K/401R/404L/670R, 138R/168T/311G/336S/340W/388K/401R/404L, 138R/172G/311G/336S/340W/388K/401R/404L, 138R/311G/336S/340W/379A/388K/401R/404L, 138R/311G/336S/340W/388K/392M/401R/404L, 138R/311G/336S/340W/388K/401R/404L/419R, 138R/311G/336S/340W/388K/392R/401R/404L, 138R/311G/336S/340W/388K/392A/401R/404L, 57G/138R/311G/336S/340W/388K/401R/404L, 138R/168S/311G/336S/340W/388K/401R/404L, 138R/164W/311G/336S/340W/388K/401R/404L, 138R/311G/336S/340W/388K/401R/404L/670Q, 138R/311G/336S/340W/388K/401R/404L/810K, 138R/311G/336S/340W/382G/388K/401R/404L, 138R/311G/336S/340W/388K/401R/404L/659R, 54H/138R/311G/336S/340W/388K/401R/404L, 138R/311G/336S/340W/388K/401R/404L/419S, 138R/172E/173Y/311G/336S/340W/388K/401R/404L/514S/659R/660H, 138R/170Q/171E/172E/311G/336S/340W/388K/401R/404L/514S/659R, 138R/173Y/311G/336S/340W/388K/401R/404L/514S/659R/660H, 138R/173Y/311G/336S/340W/388K/401R/404L/514S/659R/810K, 138R/170Q/311G/336S/388K/401R/404L/514S/659R/660H/810K, 138R/170Q/171E/173Y/311G/388K/401R/404L/514S/659R/660H, 57G/138R/171E/173Y/311G/336S/340W/388K/401R/404L/514S/659R/660H/810K, 138R/170Q/173Y/311G/336S/340W/388K/401R/404L/514S/659R/660H/810K, 57G/138R/171E/172K/311G/388K/401R/404L/514S/659R, 138R/173Y/311G/336S/340W/388K/401R/404L/514S, 138R/171E/172E/173Y/311G/336S/340W/388K/401R/404L/514S/659R/660H, 57G/138R/170Q/171E/173Y/311G/336S/340W/388K/401R/404L/514S/659R/660H/810K, 57G/138R/173Y/311G/336S/340W/388K/401R/404L/514S, 138R/172K/173D/311G/336S/340W/388K/401R/404L/514S/857V, 57G/138R/173Y/311G/336S/388K/401R/404L/514S, 138R/170Q/171E/173Y/311G/340W/388K/401R/404L/514S, 138R/171E/311G/336S/340W/388K/401R/404L/514S, 57G/138R/170Q/173Y/311G/340W/388K/401R/404L/514S, 138R/170Q/311G/340W/388K/401R/404L, 138R/173Y/311G/336S/340W/388K/401R/404L/514S/659R, 57G/138R/311G/336S/340W/388K/401R/404L/514S, or 138R/173Y/311G/336S/340W/388K/401R/404L/810K, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 38/138/311/340/388/401/404/514, 38/138/143/311/388/401/404/514/782, or 38/138/143/311/357/388/401/404, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set, or amino acid residues 38G/138R/311G/340W/388K/401R/404L/514S, 38G/138R/143V/311G/388K/401R/404L/514S/782V, or 38G/138R/143V/311G/357E/388K/401R/404L, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 138/173/207/311/336/340/388/401/404/514/659/660, 138/173/311/336/340/388/401/404/419/514/659/660, 138/173/207/311/388/401/404/514/659/660, 138/166/169/173/311/336/340/388/401/404/514/659/660, 138/173/207/311/336/340/388/401/404/514/659/660/670, 138/166/173/311/388/401/404/514/659/660, 138/173/207/311/336/340/388/401/404/419/514/659/660/670, 138/173/207/311/382/388/401/404/419/514/659/660/670, 138/173/207/311/336/340/382/388/401/404/514/659/660, 138/173/311/336/340/388/401/404/514/659/660/670, 138/173/311/340/388/401/404/514/659/660/670, 138/173/311/336/388/401/404/514/659/660/670, 138/173/311/336/340/382/388/401/404/514/659/660, 127/138/141/173/207/311/340/382/388/401/404/514/659/660/670, 138/173/207/311/340/382/388/401/404/419/514/659/660/670, 138/173/311/336/340/388/401/404/419/514/659/660/670, 138/166/168/173/207/311/388/401/404/419/514/659/660/670, 138/166/173/311/336/388/401/404/514/659/660, or 138/173/207/311/388/401/404/514/659/660/670, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set, or amino acid residues 138R/173Y/207S/311G/336S/340W/388K/401R/404L/514S/659R/660H, 138R/173Y/311G/336S/340W/388K/401R/404L/419R/514S/659R/660H, 138R/173Y/207S/311G/388K/401R/404L/514S/659R/660H, 138R/166R/169L/173Y/311G/336S/340W/388K/401R/404L/514S/659R/660H, 138R/173Y/207S/311G/336S/340W/388K/401R/404L/514S/659R/660H/670R, 138R/166R/173Y/311G/388K/401R/404L/514S/659R/660H, 138R/173Y/207S/311G/336S/340W/388K/401R/404L/419R/514S/659R/660H/670R, 138R/173Y/207S/311G/382G/388K/401R/404L/419R/514S/659R/660H/670R, 138R/173Y/207S/311G/336S/340W/382G/388K/401R/404L/514S/659R/660H, 138R/173Y/311G/336S/340W/388K/401R/404L/514S/659R/660H/670R, 138R/173Y/311G/340W/388K/401R/404L/514S/659R/660H/670R, 138R/173Y/311G/336S/388K/401R/404L/514S/659R/660H/670R, 138R/173Y/311G/336S/340W/382G/388K/401R/404L/514S/659R/660H, 127G/138R/141A/173Y/207S/311G/340W/382G/388K/401R/404L/514S/659R/660H/670R, 138R/173Y/207S/311G/340W/382G/388K/401R/404L/419R/514S/659R/660H/670R, 138R/173Y/311G/336S/340W/388K/401R/404L/419R/514S/659R/660H/670R, 138R/166R/168V/173Y/207S/311G/388K/401R/404L/419R/514S/659R/660H/670R, 138R/166V/173Y/311G/336S/388K/401R/404L/514S/659R/660H, or 138R/173Y/207S/311G/388K/401R/404L/514S/659R/660H/670R, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 38/138/173/311/336/340/388/401/404/437/514/659/660/779, 38/138/173/311/336/340/388/401/404/437/514/659/660, 38/138/173/311/336/340/388/401/404/514/659/660, 38/138/173/207/311/336/340/388/401/404/437/514/659/660/779, 138/173/311/336/340/388/401/404/514/517/659/660/779, 38/138/173/311/336/340/388/401/404/437/514/527/659/660/664, 38/138/173/207/311/336/340/379/388/401/404/437/514/659/660, 138/173/311/336/340/388/401/404/514/659/660/664/670/779, 38/138/173/311/336/340/388/401/404/514/659/660/779, 38/138/173/311/336/340/388/401/404/437/514/517/659/660, 138/173/311/336/340/388/401/404/437/514/659/660/779, 38/138/173/311/336/340/388/401/404/437/514/607/659/660, 38/138/173/311/336/340/379/388/401/404/514/659/660/779, or 138/173/311/336/340/388/401/404/514/517/659/660/670/779, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set, or amino acid residues 38G/138R/173Y/311G/336S/340W/388K/401R/404L/437P/514S/659R/660H/779R, 38G/138R/173Y/311G/336S/340W/388K/401R/404L/437P/514S/659R/660H, 38G/138R/173Y/311G/336S/340W/388K/401R/404L/514S/659R/660H, 38G/138R/173Y/207S/311G/336S/340W/388K/401R/404L/437P/514S/659R/660H/779R, 138R/173Y/311G/336S/340W/388K/401R/404L/514S/517R/659R/660H/779R, 38G/138R/173Y/311G/336S/340W/388K/401R/404L/437P/514S/527E/659R/660H/664K, 38G/138R/173Y/207S/311G/336S/340W/379S/388K/401R/404L/437P/514S/659R/660H, 138R/173Y/311G/336S/340W/388K/401R/404L/514S/659R/660H/664K/670R/779R, 38G/138R/173Y/311G/336S/340W/388K/401R/404L/514S/659R/660H/779R, 38G/138R/173Y/311G/336S/340W/388K/401R/404L/437P/514S/517R/659R/660H, 138R/173Y/311G/336S/340W/388K/401R/404L/437P/514S/659R/660H/779R, 38G/138R/173Y/311G/336S/340W/388K/401R/404L/437P/514S/607Y/659R/660H, 38G/138R/173Y/311G/336S/340W/379S/388K/401R/404L/514S/659R/660H/779R, 138R/173Y/311G/336S/340W/388K/401R/404L/514S/517R/659R/660H/670R/779R, 38G/138R/173Y/311G/336S/340W/388K/401R/404L/437P/514S/659R/660H/779R, 38G/138R/173Y/311G/336S/340W/388K/401R/404L/437P/514S/659R/660H, 38G/138R/173Y/311G/336S/340W/388K/401R/404L/514S/659R/660H, 38G/138R/173Y/207S/311G/336S/340W/388K/401R/404L/437P/514S/659R/660H/779R, 138R/173Y/311G/336S/340W/388K/401R/404L/514S/517R/659R/660H/779R, 38G/138R/173Y/311G/336S/340W/388K/401R/404L/437P/514S/527E/659R/660H/664K, 38G/138R/173Y/207S/311G/336S/340W/379S/388K/401R/404L/437P/514S/659R/660H, 138R/173Y/311G/336S/340W/388K/401R/404L/514S/659R/660H/664K/670R/779R, 38G/138R/173Y/311G/336S/340W/388K/401R/404L/L514S/659R/660H/779R, 38G/138R/173Y/311G/336S/340W/388K/401R/404L/437P/514S/517R/659R/660H, 138R/173Y/311G/336S/340W/388K/401R/404L/437P/514S/659R/660H/779R, 38G/138R/173Y/311G/336S/340W/388K/401R/404L/437P/514S/607Y/659R/660H, 38G/138R/173Y/311G/336S/340W/379S/388K/401R/404L/514S/659R/660H/779R, or 138R/173Y/311G/336S/340W/388K/401R/404L/514S/517R/659R/660H/670R/779R, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 138/173/311/336/340/357/388/394/401/404/514/659/660/664, or 173/357/394/401/404/514/659/660/664, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set, or amino acid residues 138R/173Y/311G/336S/340W/357E/388K/394K/401R/404L/514S/659R/660H/664P, or 173Y/357E/394K/401R/404S/514S/659R/660H/664P, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 38/138/173/311/336/340/388/401/404/437/514/659/660/720/779, 38/138/173/196/311/336/340/388/401/404/437/514/659/660/779, 38/138/173/311/336/340/388/401/404/437/514/609/659/660/779, 38/138/173/246/311/336/340/388/401/404/437/514/659/660/779, 38/138/173/185/311/336/340/388/401/404/437/514/659/660/779, 38/138/173/311/336/340/388/401/404/437/514/594/659/660/779, 38/138/173/212/311/336/340/388/401/404/437/514/659/660/779, 38/138/173/311/336/340/388/401/404/437/494/514/659/660/779, 38/138/173/311/336/340/375/388/401/404/437/514/659/660/779, 38/138/173/311/336/340/388/401/404/437/514/659/660/721/779, 38/72/138/173/311/336/340/388/401/404/437/514/659/660/779, 38/138/173/311/336/340/388/401/404/437/514/659/660/779/867, 38/138/160/173/311/336/340/388/401/404/437/514/659/660/779, 38/138/173/311/336/340/388/401/404/437/514/659/660/718/779, 38/138/173/187/311/336/340/388/401/404/437/514/659/660/779, 38/138/170/311/336/340/388/401/404/437/514/659/660/779, 38/138/173/311/336/340/388/401/404/437/514/615/659/660/779, 38/137/173/311/336/340/388/401/404/437/514/659/660/779, 38/138/173/311/336/340/388/401/404/437/514/659/660/779/854, 38/131/138/173/311/336/340/388/401/404/437/514/659/660/779, 138/173/185/187/311/336/340/388/401/404/514/659/660/779, 38/138/173/185/187/311/336/340/388/401/404/437/514/659/660/779, 38/138/173/311/336/340/388/401/404/514/609/659/660/779, 138/173/311/336/340/388/401/404/437/514/609/659/660/779, 138/173/185/187/311/336/340/388/401/404/514/609/659/660/779, 38/138/173/311/336/340/388/401/404/514/609/659/660/720/779, 38/138/173/185/187/311/336/340/388/401/404/437/514/609/659/660/779, 38/138/173/185/187/311/336/340/388/401/404/514/659/660/779, 38/138/173/311/336/340/388/401/404/514/659/660/721/779, 38/138/173/311/336/340/388/401/404/437/514/609/659/660/720/779, 38/138/173/185/187/246/311/336/340/388/401/404/437/514/659/660/779, or 38/138/173/185/187/311/336/340/388/401/404/514/609/659/660/720/779, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set, or amino acid residues 38G/138R/173Y/311G/336S/340W/388K/401R/404L/437P/514S/659R/660H/720A/779R, 38G/138R/173Y/196C/311G/336S/340W/388K/401R/404L/437P/514S/659R/660H/779R, 38G/138R/173Y/311G/336S/340W/388K/401R/404L/437P/514S/609W/659R/660H/779R, 38G/138R/173Y/196V/311G/336S/340W/388K/401R/404L/437P/514S/659R/660H/779R, 38G/138R/173Y/246V/311G/336S/340W/388K/401R/404L/437P/514S/659R/660H/779R, 38G/138R/173Y/185L/311G/336S/340W/388K/401R/404L/437P/514S/659R/660H/779R, 38G/138R/173Y/311G/336S/340W/388K/401R/404L/437P/514S/594G/659R/660H/779R, 38G/138R/173Y/212Y/311G/336S/340W/388K/401R/404L/437P/514S/659R/660H/779R, 38G/138R/173Y/311G/336S/340W/388K/401R/404L/437P/494G/514S/659R/660H/779R, 38G/138R/173Y/196A/311G/336S/340W/388K/401R/404L/437P/514S/659R/660H/779R, 38G/138R/173Y/212R/311G/336S/340W/388K/401R/404L/437P/514S/659R/660H/779R, 38G/138R/173Y/311G/336S/340W/375F/388K/401R/404L/437P/514S/659R/660H/779R, 38G/138R/173Y/311G/336S/340W/388K/401R/404L/437P/514S/659R/660H/721T/779R, 38G/138R/173Y/311G/336S/340W/388K/401R/404L/437P/514S/659R/660H/721A/779R, 38G/72R/138R/173Y/311G/336S/340W/388K/401R/404L/437P/514S/659R/660H/779R, 38G/138R/173Y/185I/311G/336S/340W/388K/401R/404L/437P/514S/659R/660H/779R, 38G/138R/173Y/311G/336S/340W/388K/401R/404L/437P/514S/659R/660H/721S/779R, 38G/138R/173Y/311G/336S/340W/388K/401R/404L/437P/514S/659R/660H/779R/867Q, 38G/138R/173Y/311G/336S/340W/388K/401R/404L/437P/514S/659R/660H/721Y/779R, 38G/138R/160F/173Y/311G/336S/340W/388K/401R/404L/437P/514S/659R/660H/779R, 38G/138R/173Y/311G/336S/340W/388K/401R/404L/437P/514S/659R/660H/718F/779R, 38G/138R/173Y/311G336S/K340W/388K/401R/404L/437P/514S/609F/659R/660H/779R, 38G/138R/173Y/311G/336S/340W/388K/401R/404L/437P/514S/659R/660H/720S/779R, 38G/138R/173Y/187L/311G/336S/340W/388K/401R/404L/437P/514S/659R/660H/779R, 38G/138R/173Y/311G/336S/340W/388K/401R/404L/437P/514S/659R/660H/718V/779R, 38G/138R/170L/311G/336S/340W/388K/401R/404L/437P/514S/659R/660H/779R, 38G/138R/173Y/311G/336S/340W/388K/401R/404L/437P/514S/609E/659R/660H/779R, 38G/138R/173Y/311G/336S/340W/388K/401R/404L/437P/514S/659R/660H/718S/779R, 38G/138R/173Y/311G/336S/340W/388K/401R/404L/437P/514S/609C/659R/660H/779R, 38G/138R/173Y/311G/336S/340W/388K/401R/404L/437P/514S/609P/659R/660H/779R, 38G/138R/173Y/311G/336S/340W/388K/401R/404L/437P/514S/659R/660H/721M/779R, 38G/138R/173Y/311G/336S/340W/388K/401R/404L/437P/514S/615A/659R/660H/779R, 38G/138R/173Y/187H/311G/336S/340W/388K/401R/404L/437P/514S/659R/660H/779R, 38G/138R/173Y/196S/311G/336S/340W/388K/401R/404L/437P/514S/659R/660H/779R, 38G/138R/173Y/311G/336S/340W/388K/401R/404L/437P/514S/659R/660H/721E/779R, 38G/138R/173Y/311G/336S/340W/388K/401R/404L/437P/514S/659R/660H/718Y/779R, 38G/137G/173Y/311G/336S/340W/388K/401R/404L/437P/514S/659R/660H/779R, 38G/138R/173Y/187G/311G/336S/340W/388K/401R/404L/437P/514S/659R/660H/779R, 38G/138R/173Y/311G/336S/340W/388K/401R/404L/437P/514S/659R/660H/720G/779R, 38G/138R/173Y/311G/336S/340W/388K/401R/404L/437P/514S/659R/660H/720P/779R, 38G/138R/173Y/311G/336S/340W/388K/401R/404L/437P/514S/659R/660H/779R/854T, 38G/138R/173Y/311G/336S/340W/388K/401R/404L/437P/514S/659R/660H/720I/779R, 38G/138R/160L/173Y/311G/336S/340W/388K/401R/404L/437P/514S/659R/660H/779R, 38G/131T/138R/173Y/311G/336S/340W/388K/401R/404L/437P/514S/659R/660H/779R, 138R/173Y/185L/187H/311G/336S/340W/388K/401R/404L/514S/659R/660H/779R, 38G/138R/173Y/185L/187H/311G/336S/340W/388K/401R/404L/437P/514S/659R/660H/779R, 38G/138R/173Y/185L/187L/311G/336S/340W/388K/401R/404L/437P/514S/659R/660H/779R, 38G/138R/173Y/311G/336S/340W/388K/401R/404L/514S/609C/659R/660H/779R, 138R/173Y/311G/336S/340W/388K/401R/404L/437P/514S/609P/659R/660H/779R, 138R/173Y/185L/187H/311G/336S/340W/388K/401R/404L/514S/609C/659R/660H/779R, 38G/138R/173Y/311G/336S/340W/388K/401R/404L/514S/609P/659R/660H/720A/779R, 38G/138R/173Y/185L/187L/311G/336S/340W/388K/401R/404L/437P/514S/609P/659R/660H/779R, 38G/138R/173Y/185L/187L/311G/336S/340W/388K/401R/404L/514S/659R/660H/779R, 38G/138R/173Y/185L/187H/311G/336S/340W/388K/401R/404L/514S/659R/660H/779R, 38G/138R/173Y/311G/336S/340W/388K/401R/404L/514S/609P/659R/660H/779R, 38G/138R/173Y/311G/336S/340W/388K/401R/404L/514S/659R/660H/721S/779R, 38G/138R/173Y/185L/187H/311G/336S/340W/388K/401R/404L/437P/514S/609E/659R/660H/779R, 38G/138R/173Y/311G/336S/340W/388K/401R/404L/437P/514S/609P/659R/660H/720A/779R, 38G/138R/173Y/185L/187L/246V/311G/336S/340W/388K/401R/404L/437P/514S/659R/660H/779R, or 38G/138R/173Y/185L/187H/311G/336S/340W/388K/401R/404L/514S/609P/659R/660H/720A/779R, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 138/173/311/336/340/357/388/394/401/404/514/659/660/664, 138/170/173/311/336/340/357/388/394/401/404/514/609/659/660/664, 38/138/173/185/187/311/336/340/357/388/394/401/404/514/609/659/660/664, 38/138/173/185/187/311/336/340/357/388/394/401/404/514/659/660/664, 38/138/170/311/336/340/357/388/394/401/404/514/659/660/664, 38/138/185/187/311/336/340/357/388/394/401/404/514/659/660/664/720, 138/170/173/185/187/311/336/340/357/388/394/401/404/514/609/659/660/664, 138/173/311/336/340/357/388/394/401/404/514/609/659/660/664, 138/170/173/185/187/311/336/340/357/388/394/401/404/514/609/659/660/664/721, 38/138/173/311/336/340/357/388/394/401/404/514/609/659/660/664, 38/138/170/173/246/311/336/340/357/388/394/401/404/514/609/659/660/664, 138/173/185/187/311/336/340/357/388/394/401/404/514/609/659/660/664/718, 38/138/173/185/187/311/336/340/357/388/394/401/404/514/609/659/660/664/718/720, 38/138/170/173/246/311/336/340/357/388/394/401/404/514/609/659/660/664/718, 138/173/311/336/340/357/388/394/401/404/514/659/660/664/720, 138/173/246/311/336/340/357/388/394/401/404/514/609/659/660/664, 38/138/173/185/187/246/311/336/340/357/388/394/401/404/514/659/660/664/720, 38/138/173/311/336/340/357/388/394/401/404/514/609/659/660/664/721, 38/138/173/311/336/340/357/388/394/401/404/514/659/660/664/721, 38/138/173/311/336/340/357/388/394/401/404/514/659/660/664/720/721, 38/138/170/173/311/336/340/357/388/394/401/404/514/609/659/660/664/718/720, 38/138/173/311/336/340/357/388/394/401/404/514/659/660/664/718, 38/138/173/185/187/311/336/340/357/388/394/401/404/514/609/659/660/664/718/721, 138/173/311/336/340/357/388/394/401/404/514/529/609/659/660/664, 38/138/173/311/336/340/357/388/394/401/404/514/609/659/660/664/718, 138/173/185/187/311/336/340/357/388/394/401/404/514/609/659/660/664/718/720, 138/173/185/187/311/336/340/357/388/394/401/404/514/609/659/660/664/718/721, 38/138/170/173/311/336/340/357/388/394/401/404/514/659/660/664/718/720/721, 138/170/173/185/187/311/336/340/357/388/394/401/404/514/659/660/664/718/720, 38/138/185/187/246/311/336/340/357/388/394/401/404/514/609/659/660/664/720, 138/173/311/336/340/357/388/394/401/404/514/609/659/660/664/718/720, 138/170/173/185/187/311/336/340/357/388/394/401/404/514/659/660/664/718/720/721, 38/138/170/246/311/336/340/357/388/394/401/404/514/609/659/660/664/718, 138/173/246/311/336/340/357/388/394/401/404/514/659/660/664/721, 38/138/173/311/336/340/357/388/394/401/404/514/659/660/664/718/720, or 38/138/173/311/336/340/357/388/394/401/404/514/609/659/660/664/718/720, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residues 138R/173Y/311G/336S/340W/357E/388K/394K/401R/404L/514S/659R/660H/664P, 138R/170L/173Y/311G/336S/340W/357E/388K/394K/401R/404L/514S/609P/659R/660H/664P, 38G/138R/173Y/185L/187L/311G/336S/340W/357E/388K/394K/401R/404L/514S/609C/659R/660H/664P, 38G/138R/173Y/185L/187H/311G/336S/340W/357E/388K/394K/401R/404L/514S/659R/660H/664P, 38G/138R/170L/311G/336S/340W/357E/388K/394K/401R/404L/514S/659R/660H/664P, 38G/138R/185L/187L/311G/336S/340W/357E/388K/394K/401R/404L/514S/659R/660H/664P/720A, 138R/170L/173Y/185L/187H/311G/336S/340W/357E/388K/394K/401R/404L/514S/609C/659R/660H/664P, 138R/173Y/311G/336S/340W/357E/388K/394K/401R/404L/514S/609E/659R/660H/664P, 138R/170L/173Y/185L/187H/311G/336S/340W/357E/388K/394K/401R/404L/514S/609C/659R/660H/664P/721S, 38G/138R/173Y/311G/336S/340W/357E/388K/394K/401R/404L/514S/609E/659R/660H/664P, 138R/170L/173Y/185L/187L/311G/336S/340W/357E/388K/394K/401R/404L/514S/609E/659R/660H/664P, 38G/138R/170L/173Y/246V/311G/336S/340W/357E/388K/394K/401R/404L/514S/609P/659R/660H/664P, 138R/173Y/185L/187H/311G/336S/340W/357E/388K/394K/401R/404L/514S/609E/659R/660H/664P/718S, 38G/138R/173Y/185L/187L/311G/336S/340W/357E/388K/394K/401R/404L/514S/609W/659R/660H/664P/718N/720A, 38G/138R/170L/173Y/246V/311G/336S/340W/357E/388K/394K/401R/404L/514S/609C/659R/660H/664P/718S, 138R/173Y/311G/336S/340W/357E/388K/394K/401R/404L/514S/659R/660H/664P/720A, 138R/173Y/246V/311G/336S/340W/357E/388K/394K/401R/404L/514S/609E/659R/660H/664P, 38G/138R/173Y/185L/187L/246V/311G/336S/340W/357E/388K/394K/401R/404L/514S/659R/660H/664P/720A, 38G/138R/173Y/311G/336S/340W/357E/388K/394K/401R/404L/514S/609C/659R/660H/664P/721S, 38G/138R/173Y/311G/336S/340W/357E/388K/394K/401R/404L/514S/659R/660H/664P/721S, 38G/138R/173Y/311G/336S/340W/357E/388K/394K/401R/404L/514S/659R/660H/664P/720A/721S, 38G/138R/170L/173Y/311G/336S/340W/357E/388K/394K/401R/404L/514S/609C/659R/660H/664P/718S/720A, 38G/138R/173Y/311G/336S/340W/357E/388K/394K/401R/404L/514S/659R/660H/664P/718S, 38G/138R/170L/173Y/246V/311G/336S/340W/357E/388K/394K/401R/404L/514S/609P/659R/660H/664P/718S, 38G/138R/173Y/185L/187L/311G/336S/340W/357E/388K/394K/401R/404L/514S/609P/659R/660H/664P/718S/721S, 138R/173Y/311G/336S/340W/357E/388K/394K/401R/404L/514S/529R/609E/659R/660H/664P, 38G/138R/173Y/311G/336S/340W/357E/388K/394K/401R/404L/514S/609P/659R/660H/664P/718S, 138R/173Y/185L/187L/311G/336S/340W/357E/388K/394K/401R/404L/514S/609C/659R/660H/664P/718S/720A, 138R/173Y/185L/187H/311G/336S/340W/357E/388K/394K/401R/404L/514S/609C/659R/660H/664P/718S/721S, 38G/138R/170L/173Y/311G/336S/340W/357E/388K/394K/401R/404L/514S/659R/660H/664P/718S/720A/721S, 138R/170L/173Y/185L/187L/311G/336S/340W/357E/388K/394K/401R/404L/514S/659R/660H/664P/718S/720A, 38G/138R/185L/187L/246V/311G/336S/340W/357E/388K/394K/401R/404L/514S/609E/659R/660H/664P/720A, 138R/173Y/311G/336S/340W/357E/388K/394K/401R/404L/514S/609E/659R/660H/664P/718S/720A, 138R/170L/173Y/185L/187H/311G/336S/340W/357E/388K/394K/401R/404L/514S/659R/660H/664P/718S/720A/721S, 38G/138R/170L/246V/311G/336S/340W/357E/388K/394K/401R/404L/514S/609E/659R/660H/664P/718S, 138R/173Y/246V/311G/336S/340W/357E/388K/394K/401R/404L/514S/659R/660H/664P/721S, 38G/138R/173Y/311G/336S/340W/357E/388K/394K/401R/404L/514S/659R/660H/664P/718S/720A, or 38G/138R/173Y/311G/336S/340W/357E/388K/394K/401R/404L/514S/609E/659R/660H/664P/718S/720A, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 38/138/143/173/311/336/340/388/401/404/514/659/660, 38/105/138/173/311/336/340/388/401/404/514/659/660, 38/138/173/311/336/340/388/401/404/514/659/660/775/779, 38/135/138/173/311/336/340/388/401/404/514/659/660, 38/100/138/173/311/336/340/388/401/404/514/659/660, 38/138/173/246/311/336/340/388/401/404/514/659/660, 38/138/173/311/336/340/388/401/404/514/659/660/779/782, 38/138/173/311/336/340/388/401/404/514/659/660/749, 38/138/173/237/311/336/340/388/401/404/514/659/660, 38/138/162/173/311/336/340/388/401/404/514/659/660, 38/83/138/173/311/336/340/388/401/404/514/659/660, 38/138/173/218/311/336/340/388/401/404/514/659/660, 38/138/173/227/311/336/340/388/401/404/514/659/660, 38/115/138/173/311/336/340/388/401/404/514/659/660, 38/138/173/274/311/336/340/388/401/404/514/659/660, 38/81/138/173/311/336/340/388/401/404/514/659/660, 38/122/138/173/311/336/340/388/401/404/514/659/660, 38/61/138/173/311/336/340/388/401/404/514/659/660, 32/38/138/173/311/336/340/388/401/404/514/659/660, 38/138/139/173/311/336/340/388/401/404/514/659/660, 38/75/138/173/311/336/340/388/401/404/514/659/660, 38/138/173/239/311/336/340/388/401/404/514/659/660, 38/138/173/257/311/336/340/388/401/404/514/659/660, 38/138/173/240/311/336/340/388/401/404/514/659/660, 38/69/138/161/173/311/336/340/388/401/404/514/659/660, 38/138/173/253/311/336/340/388/401/404/514/659/660, 14/38/138/173/311/336/340/388/401/404/514/659/660, 38/138/173/221/311/336/340/388/401/404/514/659/660, 38/84/138/173/311/336/340/388/401/404/514/659/660, 38/133/138/150/173/311/336/340/388/401/404/514/659/660, 38/60/138/173/311/336/340/388/401/404/514/659/660, 38/138/173/275/311/336/340/388/401/404/514/659/660, 38/89/138/173/311/336/340/388/401/404/514/659/660, 15/38/138/173/311/336/340/388/401/404/514/659/660, 38/138/173/254/311/336/340/388/401/404/514/659/660, 38/138/173/311/336/340/388/401/404/514/625/659/660, 14/38/67/138/173/311/336/340/388/401/404/514/659/660, 21/38/138/173/311/336/340/388/401/404/514/659/660, 38/93/138/173/311/336/340/388/401/404/514/659/660, 38/138/173/250/311/336/340/388/401/404/514/659/660, 38/138/173/217/311/336/340/388/401/404/514/659/660, 19/38/138/173/311/336/340/388/401/404/514/659/660, 38/95/138/173/311/336/340/388/401/404/514/659/660, 38/138/173/311/336/340/388/401/404/514/659/660/759, 38/62/138/173/311/336/340/388/401/404/514/659/660, or 38/138/140/173/311/336/340/388/401/404/514/659/660, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residues 38G/138R/143N/173Y/311G/336S/340W/388K/401R/404L/514S/659R/660H, 38G/138R/143Q/173Y/311G/336S/340W/388K/401R/404L/514S/659R/660H, 38G/138R/143R/173Y/311G/336S/340W/388K/401R/404L/514S/659R/660H, 38G/105R/138R/173Y/311G/336S/340W/388K/401R/404L/514S/659R/660H, 38G/138R/173Y/311G/336S/340W/388K/401R/404L/514S/659R/660H/775R/779R, 38G/135K/138R/173Y/311G/336S/340W/388K/401R/404L/514S/659R/660H, 38G/100R/138R/173Y/311G/336S/340W/388K/401R/404L/514S/659R/660H, 38G/138R/173Y/311G/336S/340W/388K/401R/404L/514S/659R/660H/775K/779R, 38G/138R/173Y/246K/311G/336S/340W/388K/401R/404L/514S/659R/660H, 38G/138R/173Y/311G/336S/340W/388K/401R/404L/514S/659R/660H/779R/782R, 38G/138R/173Y/311G/336S/340W/388K/401R/404L/514S/659R/660H/749R, 38G/138R/173Y/237L/311G/336S/340W/388K/401R/404L/514S/659R/660H, 38G/138R/162V/173Y/311G/336S/340W/388K/401R/404L/514S/659R/660H, 38G/83K/138R/173Y/311G/336S/340W/388K/401R/404L/514S/659R/660H, 38G/138R/173Y/218T/311G/336S/340W/388K/401R/404L/514S/659R/660H, 38G/138R/173Y/227V/311G/336S/340W/388K/401R/404L/514S/659R/660H, 38G/115L/138R/173Y/311G/336S/340W/388K/401R/404L/514S/659R/660H, 38G/138R/173Y/274G/311G/336S/340W/388K/401R/404L/514S/659R/660H, 38G/81C/138R/173Y/311G/336S/340W/388K/401R/404L/514S/659R/660H, 38G/122I/138R/173Y/311G/336S/340W/388K/401R/404L/514S/659R/660H, 38G/61Y/138R/173Y/311G/336S/340W/388K/401R/404L/514S/659R/660H, 32S/38G/138R/173Y/311G/336S/340W/388K/401R/404L/514S/659R/660H, 38G/138R/139R/173Y/311G/336S/340W/388K/401R/404L/514S/659R/660H, 38G/75Y/138R/173Y/311G/336S/340W/388K/401R/404L/514S/659R/660H, 38G/138R/173Y/239R/311G/336S/340W/388K/401R/404L/514S/659R/660H, 38G/138R/173Y/257L/311G/336S/340W/388K/401R/404L/514S/659R/660H, 38G/138R/173Y/240M/311G/336S/340W/388K/401R/404L/514S/659R/660H, 38G/69S/138R/161R/173Y/311G/336S/340W/388K/401R/404L/514S/659R/660H, 38G/138R/173Y/253Y/311G/336S/340W/388K/401R/404L/514S/659R/660H, 14Y/38G/138R/173Y/311G/336S/340W/388K/401R/404L/514S/659R/660H, 38G/138R/173Y/221C/311G/336S/340W/388K/401R/404L/514S/659R/660H, 38G/84A/138R/173Y/311G/336S/340W/388K/401R/404L/514S/659R/660H, 38G/115V/138R/173Y/311G/336S/340W/388K/401R/404L/514S/659R/660H, 38G/133C/138R/150Q/173Y/311G/336S/340W/388K/401R/404L/514S/659R/660H, 38G/60L/138R/173Y/311G/336S/340W/388K/401R/404L/514S/659R/660H, 38G/60G/138R/173Y/311G/336S/340W/388K/401R/404L/514S/659R/660H, 32V/38G/138R/173Y/311G/336S/340W/388K/401R/404L/514S/659R/660H, 38G/138R/173Y/275A/311G/336S/340W/388K/401R/404L/514S/659R/660H, 38G/89Q/138R/173Y/311G/336S/340W/388K/401R/404L/514S/659R/660H, 38G/138R/173Y/257V/311G/336S/340W/388K/401R/404L/514S/659R/660H, 38G/138R/173Y/274L/311G/336S/340W/388K/401R/404L/514S/659R/660H, 15T/38G/138R/173Y/311G/336S/340W/388K/401R/404L/514S/659R/660H, 38G/138R/173Y/254S/311G/336S/340W/388K/401R/404L/514S/659R/660H, 38G/61W/138R/173Y/311G/336S/340W/388K/401R/404L/514S/659R/660H, 38G/138R/173Y/311G/336S/340W/388K/401R/404L/514S/625C/659R/660H, 14Q/38G/67R/138R/173Y/311G/336S/340W/388K/401R/404L/514S/659R/660H, 21G/38G/138R/173Y/311G/336S/340W/388K/401R/404L/514S/659R/660H, 38G/138R/173Y/254G/311G/336S/340W/388K/401R/404L/514S/659R/660H, 38G/93Y/138R/173Y/311G/336S/340W/388K/401R/404L/514S/659R/660H, 38G/138R/173Y/250R/311G/336S/340W/388K/401R/404L/514S/659R/660H, 38G/138R/173Y/217V/311G/336S/340W/388K/401R/404L/514S/659R/660H, 38G/60M/138R/173Y/311G/336S/340W/388K/401R/404L/514S/659R/660H, 38G/138R/173Y/239L/311G/336S/340W/388K/401R/404L/514S/659R/660H, 38G/75S/138R/173Y/311G/336S/340W/388K/401R/404L/514S/659R/660H, 32T/38G/138R/173Y/311G/336S/340W/388K/401R/404L/514S/659R/660H, 19E/38G/138R/173Y/311G/336S/340W/388K/401R/404L/514S/659R/660H, 38G/95Y/138R/173Y/311G/336S/340W/388K/401R/404L/514S/659R/660H, 38G/81L/138R/173Y/311G/336S/340W/388K/401R/404L/514S/659R/660H, 38G/60R/138R/173Y/311G/336S/340W/388K/401R/404L/514S/659R/660H, 38G/138R/173Y/311G/336S/340W/388K/401R/404L/514S/659R/660H/759M, 38G/62T/138R/173Y/311G/336S/340W/388K/401R/404L/514S/659R/660H, or 38G/138R/140R/173Y/311G/336S/340W/388K/401R/404L/514S/659R/660H, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 38/138/170/311/336/340/357/388/394/401/404/514/609/659/660/664/779, 38/138/170/246/311/336/340/357/388/394/401/404/514/609/659/660/664, 38/138/170/246/311/336/340/357/388/394/401/404/514/659/660/664, 38/138/170/311/336/340/357/388/394/401/404/514/609/659/660/664/775/779, 38/105/138/170/311/336/340/357/388/394/401/404/514/659/660/664, 38/138/143/170/311/336/340/357/388/394/401/404/514/609/659/660/664/775, 38/105/135/138/170/311/336/340/357/388/394/401/404/514/609/659/660/664/775, 38/111/138/170/311/336/340/357/388/394/401/404/514/609/659/660/664, or 38/138/170/311/336/340/357/388/394/401/404/514/609/659/660/664, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residue 38G/138R/170L/311G/336S/340W/357E/388K/394K/401R/404L/514S/609E/659R/660H/664P/779R, 38G/138R/170L/246K/311G/336S/340W/357E/388K/394K/401R/404L/514S/609E/659R/660H/664P, 38G/138R/170L/246K/311G/336S/340W/357E/388K/394K/401R/404L/514S/659R/660H/664P, 38G/138R/170L/311G/336S/340W/357E/388K/394K/401R/404L/514S/609E/659R/660H/664P/775K/779R, 38G/105R/138R/170L/311G/336S/340W/357E/388K/394K/401R/404L/514S/659R/660H/664P, 38G/138R/143N/170L/311G/336S/340W/357E/388K/394K/401R/404L/514S/609E/659R/660H/664P/775K, 38G/105R/135K/138R/170L/311G/336S/340W/357E/388K/394K/401R/404L/514S/609E/659R/660H/664P/775K, 38G/111K/138R/170L/311G/336S/340W/357E/388K/394K/401R/404L/514S/609E/659R/660H/664P, or 38G/138R/170L/311G/336S/340W/357E/388K/394K/401R/404L/514S/609E/659R/660H/664P, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 32/38/138/170/246/311/336/340/357/388/394/401/404/514/609/659/660/664, 38/138/170/246/311/336/340/357/388/394/401/404/514/531/533/609/659/660/664, 38/115/138/170/246/311/336/340/357/388/394/401/404/514/609/659/660/664, 38/83/138/170/246/311/336/340/357/388/394/401/404/514/609/659/660/664, 32/38/60/61/138/170/246/311/336/340/357/388/394/401/404/514/609/659/660/664, 38/55/138/170/246/311/336/340/357/388/394/401/404/514/609/659/660/664, 38/138/170/246/311/336/340/357/388/394/401/404/514/589/609/659/660/664, 38/138/170/246/311/336/340/357/388/394/401/404/514/609/659/660/664, 38/138/170/246/311/336/340/357/388/394/401/404/514/609/617/659/660/664, 38/138/170/172/246/311/336/340/357/388/394/401/404/514/609/659/660/664, 38/138/170/246/311/336/340/357/388/394/401/404/514/598/609/659/660/664, 38/56/138/170/193/246/311/336/340/357/388/394/401/404/514/609/659/660/664, 38/56/138/170/246/311/336/340/357/388/394/401/404/514/609/659/660/664, 38/138/170/246/311/336/340/357/388/394/401/404/514/536/609/659/660/664, 38/52/138/170/246/311/336/340/357/388/394/401/404/514/609/659/660/664, 17/38/138/170/246/311/336/340/357/388/394/401/404/514/609/659/660/664, 38/138/170/246/261/311/336/340/357/388/394/401/404/514/609/659/660/664, 38/78/138/170/246/311/336/340/357/388/394/401/404/514/609/659/660/664, 38/138/170/246/311/336/340/357/388/394/401/404/514/609/629/659/660/664, 38/63/138/170/246/311/336/340/357/388/394/401/404/514/609/659/660/664, 38/82/138/170/246/311/336/340/357/388/394/401/404/514/609/659/660/664, 38/138/170/207/246/311/336/340/357/388/394/401/404/514/609/659/660/664, 38/138/170/246/311/336/340/357/388/394/401/404/514/609/659/660/664/666, 38/138/150/170/246/311/336/340/357/388/394/401/404/514/609/659/660/664, 38/45/138/170/246/311/336/340/357/388/394/401/404/514/609/659/660/664, 38/135/138/170/246/311/336/340/357/388/394/401/404/514/609/659/660/664, 13/38/138/170/246/311/336/340/357/388/394/401/404/514/609/659/660/664, 33/38/138/170/246/311/336/340/357/388/394/401/404/514/609/659/660/664, or 38/138/170/245/246/311/336/340/357/388/394/401/404/514/609/659/660/664, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residues 32V/38G/138R/170L/246K/311G/336S/340W/357E/388K/394K/401R/404L/514S/609E/659R/660H/664P, 38G/138R/170L/246K/311G/336S/340W/357E/388K/394K/401R/404L/514S/531Q/533S/609E/659R/660H/664P, 38G/115L/138R/170L/246K/311G/336S/340W/357E/388K/394K/401R/404L/514S/609E/659R/660H/664P, 38G/83K/138R/170L/246K/311G/336S/340W/357E/388K/394K/401R/404L/514S/609E/659R/660H/664P, 32V/38G/60M/61W/138R/170L/246K/311G/336S/340W/357E/388K/394K/401R/404L/514S/609E/659R/660H/664P, 38G/55D/138R/170L/246K/311G/336S/340W/357E/388K/394K/401R/404L/514S/609E/659R/660H/664P, 38G/138R/170L/246K/311G/336S/340W/357E/388K/394K/401R/404L/514S/589C/609E/659R/660H/664P, 38G/138R/170L/246K/311G/336S/340W/357P/388K/394K/401R/404L/514S/609E/659R/660H/664P, 38G/138R/170L/246K/311G/336S/340W/357E/388K/394K/401R/404L/514S/609E/617R/659R/660H/664P, 38G/138R/170L/172G/246K/311G/336S/340W/357E/388K/394K/401R/404L/514S/609E/659R/660H/664P, 38G/138R/170L/246K/311G/336S/340W/357E/388K/394K/401R/404L/514S/598A/609E/659R/660H/664P, 38G/56G/138R/170L/193G/246K/311G/336S/340W/357E/388K/394K/401R/404L/514S/609E/659R/660H/664P, 38G/56T/138R/170L/246K/311G/336S/340W/357E/388K/394K/401R/404L/514S/609E/659R/660H/664P, 38G/138R/170L/246K/311G/336S/340W/357E/388K/394K/401R/404L/514S/536V/609E/659R/660H/664P, 38G/52T/138R/170L/246K/311G/336S/340W/357E/388K/394K/401R/404L/514S/609E/659R/660H/664P, 17N/38G/138R/170L/246K/311G/336S/340W/357E/388K/394K/401R/404L/514S/609E/659R/660H/664P, 38G/56N/138R/170L/246K/311G/336S/340W/357E/388K/394K/401R/404L/514S/609E/659R/660H/664P, 38G/138R/170L/246K/311G/336S/340W/357E/388K/394K/401R/404L/514S/598K/609E/659R/660H/664P, 38G/138R/170L/246K/261M/311G/336S/340W/357E/388K/394K/401R/404L/514S/609E/659R/660H/664P, 38G/56E/138R/170L/246K/311G/336S/340W/357E/388K/394K/401R/404L/514S/609E/659R/660H/664P, 38G/55S/138R/170L/246K/311G/336S/340W/357E/388K/394K/401R/404L/514S/609E/659R/660H/664P, 38G/138R/170L/246K/311G/336S/340W/357E/388K/394K/401R/404L/514S/536E/609E/659R/660H/664P, 38G/138R/170L/246K/311G/336S/340W/357E/388K/394K/401R/404L/514S/598L/609E/659R/660H/664P, 38G/78S/138R/170L/246K/311G/336S/340W/357E/388K/394K/401R/404L/514S/609E/659R/660H/664P, 38G/56V/138R/170L/246K/311G/336S/340W/357E/388K/394K/401R/404L/514S/609E/659R/660H/664P, 38G/138R/170L/246K/311G/336S/340W/357E/388K/394K/401R/404L/514S/609E/629R/659R/660H/664P, 38G/138R/170L/246K/311G/336S/340W/357E/388K/394K/401R/404L/514S/598R/609E/659R/660H/664P, 38G/63S/138R/170L/246K/311G/336S/340W/357E/388K/394K/401R/404L/514S/609E/659R/660H/664P, 38G/82S/138R/170L/246K/311G/336S/340W/357E/388K/394K/401R/404L/514S/609E/659R/660H/664P, 38G/138R/170L/207S/246K/311G/336S/340W/357E/388K/394K/401R/404L/514S/609E/659R/660H/664P, 38G/138R/170L/246K/311G/336S/340W/357E/388K/394K/401R/404L/514S/598V/609E/659R/660H/664P, 38G/138R/170L/246K/311G/336S/340W/357E/388K/394K/401R/404L/514S/609E/659R/660H/664P/666M, 38G/138R/150Y/170L/246K/311G/336S/340W/357E/388K/394K/401R/404L/514S/609E/659R/660H/664P, 38G/45K/138R/170L/246K/311G/336S/340W/357E/388K/394K/401R/404L/514S/609E/659R/660H/664P, 38G/55W/138R/170L/246K/311G/336S/340W/357E/388K/394K/401R/404L/514S/609E/659R/660H/664P, 38G/135G/138R/170L/246K/311G/336S/340W/357E/388K/394K/401R/404L/514S/609E/659R/660H/664P, 13N/38G/138R/170L/246K/311G/336S/340W/357E/388K/394K/401R/404L/514S/609E/659R/660H/664P, 33E/38G/138R/170L/246K/311G/336S/340W/357E/388K/394K/401R/404L/514S/609E/659R/660H/664P, or 38G/138R/170L/245C/246K/311G/336S/340W/357E/388K/394K/401R/404L/514S/609E/659R/660H/664P, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 32/38/138/170/207/245/246/311/336/340/357/388/394/401/404/514/609/629/659/660/664, 17/32/38/56/138/150/170/246/311/336/340/357/388/394/401/404/514/598/609/659/660/664, 32/38/56/138/170/246/311/336/340/357/388/394/401/404/514/609/629/659/660/664, 32/38/138/170/246/311/336/340/357/388/394/401/404/514/536/609/659/660/664, 32/38/138/170/185/246/311/336/340/357/388/394/401/404/514/609/659/660/664, 32/38/56/138/150/170/246/311/336/340/357/388/394/401/404/514/609/659/660/664, 17/32/38/56/138/150/170/207/245/246/311/336/340/357/388/394/401/404/514/598/609/617/629/659/660/664, 32/38/56/138/150/170/246/311/336/340/357/388/394/401/404/514/536/598/609/659/660/664, 32/38/56/138/170/246/311/336/340/357/388/394/401/404/514/536/609/659/660/664, 32/38/138/170/185/246/311/336/340/357/388/394/401/404/514/609/659/660/664/666, 32/38/138/150/170/246/311/336/340/357/388/394/401/404/514/609/659/660/664, or 32/38/138/143/150/170/246/311/336/340/357/388/394/401/404/514/609/659/660/664/782, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residues 32V/38G/138R/170L/207S/245C/246K/311G/336S/340W/357E/388K/394K/401R/404L/514S/609E/629R/659R/660H/664P, 17N/32V/38G/56E/138R/150Y/170L/246K/311G/336S/340W/357E/388K/394K/401R/404L/514S/598A/609E/659R/660H/664P, 32V/38G/56E/138R/170L/246K/311G/336S/340W/357E/388K/394K/401R/404L/514S/609E/629R/659R/660H/664P, 32V/38G/138R/170L/246K/311G/336S/340W/357E/388K/394K/401R/404L/514S/536V/609E/659R/660H/664P, 32V/38G/138R/170L/185P/246K/311G/336S/340W/357E/388K/394K/401R/404L/514S/609E/659R/660H/664P, 32V/38G/56T/138R/150Y/170L/246K/311G/336S/340W/357E/388K/394K/401R/404L/514S/609E/659R/660H/664P, 17N/32V/38G/56E/138R/150Y/170L/207S/245C/246K/311G/336S/340W/357E/388K/394K/401R/404L/514S/598A/609E/617R/629R/659R/660H/664P, 32V/38G/56E/138R/150Y/170L/246K/311G/336S/340W/357E/388K/394K/401R/404L/514S/536V/598K/609E/659R/660H/664P, 32V/38G/56V/138R/170L/246K/311G/336S/340W/357E/388K/394K/401R/404L/514S/536V/609E/659R/660H/664P, 32V/38G/138R/170L/185P/246K/311G/336S/340W/357E/388K/394K/401R/404L/514S/609E/659R/660H/664P/666M, 32V/38G/138R/150Y/170L/246K/311G/336S/340W/357E/388K/394K/401R/404L/514S/609E/659R/660H/664P, or 32V/38G/138R/143N/150Y/170L/246V/311G/336S/340W/357E/388K/394K/401R/404L/514S/609E/659R/660H/664P/782V, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 32/38/56/138/143/170/246/311/336/340/357/388/394/401/404/514/609/629/659/660/664, 32/38/56/138/170/246/311/336/340/357/388/394/401/404/424/514/609/629/648/659/660/664/782, 32/38/56/138/170/246/311/336/340/357/388/394/401/404/514/609/629/660/664/782, 32/38/56/138/150/170/246/311/336/340/357/388/394/401/404/514/609/659/660/664/782, 32/38/56/138/170/246/311/336/340/357/388/394/401/404/514/609/629/659/660/664/721/782, 32/38/56/138/170/246/311/336/340/357/388/394/401/404/514/609/629/659/660/664/782, 32/38/56/138/170/196/246/311/336/340/357/388/394/401/404/514/609/629/660/664/782, or 32/38/56/138/170/246/311/336/340/357/388/394/401/404/514/609/629/659/660/664/720/721/782, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residues 32V/38G/56E/138R/143N/170L/246K/311G/336S/340W/357E/388K/394K/401R/404L/514S/609E/629R/659R/660H/664P, 32V/38G/56E/138R/170L/246R/311G/336S/340W/357E/388K/394K/401R/404L/424A/514S/609E/629R/648F/659R/660H/664P/782V, 32V/38G/56E/138R/170L/246R/311G/336S/340W/357E/388K/394K/401R/404L/514S/609E/629R/660H/664P/782V, 32V/38G/56T/138R/150Y/170L/246K/311G/336S/340W/357E/388K/394K/401R/404L/514S/609E/659R/660W/664P/782V, 32V/38G/56E/138R/170L/246K/311G/336S/340W/357E/388K/394K/401R/404L/514S/609E/629R/659R/660H/664P/721T/782V, 32V/38G/56E/138R/170L/246K/311G/336S/340W/357E/388K/394K/401R/404L/514S/609E/629R/659R/660H/664P/782V, 32V/38G/56E/138R/170L/196G/246K/311G/336S/340W/357E/388K/394K/401R/404L/514S/609E/629R/660H/664P/782V, or 32V/38G/56E/138R/170L/246K/311G/336S/340W/357E/388K/394K/401R/404L/514S/609E/629R/659R/660H/664P/720S/721A/782V, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 32/38/56/138/143/150/170/246/311/336/340/357/388/394/401/404/514/609/660/664/782, 32/38/56/138/150/166/170/171/246/336/340/357/388/394/401/404/514/609/659/660/664, 32/38/56/138/143/150/170/246/311/336/340/357/388/394/401/404/514/609/659/660/664/721, 32/38/56/138/150/170/246/311/336/340/357/388/394/401/404/514/609/659/660/664/782, or 32/38/56/138/143/150/170/171/246/311/336/340/357/388/394/401/404/514/609/660/664/782, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residues 32V/38G/56T/138R/143N/150Y/170L/246K/311G/336S/340W/357E/388K/394K/401R/404L/514S/609E/660H/664P/782V, 32V/38G/56T/138R/150Y/166T/170L/171E/246R/336S/340W/357E/388K/394K/401R/404L/514S/609E/659R/660H/664P, 32V/38G/56T/138R/143N/150Y/170L/246K/311G/336S/340W/357E/388K/394K/401R/404L/514S/609E/659R/660H/664P/721T, 32V/38G/56T/138R/150Y/170L/246K/311G/336S/340W/357E/388K/394K/401R/404L/514S/609E/659R/660H/664P/782V, or 32V/38G/56T/138R/143N/150Y/170L/171E/246K/311G/336S/340W/357E/388K/394K/401R/404L/514S/609E/660H/664P/782V, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 32/38/56/138/150/170/246/311/336/340/357/367/388/394/401/404/514/609/659/660/664/782, 32/38/56/138/150/170/246/311/336/340/357/382/388/394/401/404/514/609/659/660/664/782, 32/38/56/138/150/170/246/311/336/340/357/388/394/401/404/514/609/659/660/664/670/782, 32/38/56/138/150/170/246/311/336/340/357/388/394/401/404/514/609/659/660/664/672/782, 32/38/56/138/150/170/246/311/336/340/357/388/394/401/404/514/609/659/660/664/782, 32/38/56/138/150/170/246/311/336/340/357/388/394/401/404/514/609/659/660/664/693/782, 32/38/56/138/150/170/246/311/336/340/357/388/394/397/401/404/514/609/659/660/664/782, 32/38/56/138/150/170/246/311/336/340/357/375/388/394/401/404/514/609/659/660/664/782, 32/38/56/137/138/150/170/246/311/336/340/357/382/388/394/401/404/514/609/659/660/664, 32/38/56/138/150/170/246/311/336/340/357/388/394/401/404/514/609/655/659/660/664/782, 32/38/56/137/138/150/170/246/311/336/340/357/382/388/392/394/401/404/514/609/659/660/664/782, 32/38/56/138/150/170/246/311/336/340/357/388/391/394/401/404/514/609/659/660/664/782, 32/38/56/137/138/150/170/246/311/336/340/357/388/394/401/404/514/609/655/659/660/664/721/763/782, 32/38/56/138/150/170/246/311/336/340/357/388/394/401/404/514/609/659/660/664/696/782, 32/38/56/138/150/170/246/311/336/340/357/388/394/401/404/514/609/659/660/663/664/782, 32/38/56/137/138/150/170/246/311/336/340/357/382/388/394/401/404/514/609/659/660/664/782, 32/38/56/137/138/150/170/246/311/336/340/357/382/388/392/394/401/404/514/609/650/655/659/660/664, 32/38/56/137/138/150/170/246/311/336/340/357/382/388/394/401/404/514/609/629/659/660/664/672/782, 32/38/56/138/150/170/246/311/336/340/357/388/394/401/404/514/609/616/659/660/664/782, 32/38/56/138/150/170/246/311/336/340/357/388/394/401/404/514/609/659/660/664/690/782, 32/38/56/138/150/170/179/246/311/336/340/357/388/394/401/404/514/609/659/660/664/782, 32/38/56/137/138/150/170/246/311/336/340/357/388/394/401/404/514/609/659/660/664/782, 32/38/56/138/150/170/246/311/336/340/357/382/388/394/401/404/514/609/659/660/664/670/782, 32/38/56/138/150/170/246/311/336/340/357/388/394/401/404/514/609/659/660/664/670/672/782, 32/38/56/138/150/170/246/311/336/340/357/388/394/401/404/514/609/629/659/660/664/782, or 32/38/56/138/150/170/246/311/336/340/357/388/394/401/404/514/609/655/659/660/664/670/782, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residues 32V/38G/56T/138R/150Y/170L/246K/311G/336S/340W/357E/367P/388K/394K/401R/404L/514S/609E/659R/660W/664P/782V, 32V/38G/56T/138R/150Y/170L/246K/311G/336S/340W/357E/382R/388K/394K/401R/404L/514S/609E/659R/660W/664P/782V, 32V/38G/56T/138R/150Y/170L/246K/311G/336S/340W/357E/388K/394K/401R/404L/514S/609E/659R/660W/664P/670R/782V, 32V/38G/56T/138R/150Y/170L/246K/311G/336S/340W/357E/382L/388K/394K/401R/404L/514S/609E/659R/660W/664P/782V, 32V/38G/56T/138R/150Y/170L/246K/311G/336S/340W/357E/388K/394K/401R/404L/514S/609E/659R/660W/664P/672P/782V, 32V/38G/56T/138R/150Y/170L/246K/311G/336S/340W/357P/388K/394K/401R/404L/514S/609E/659R/660W/664P/782V, 32V/38G/56T/138R/150Y/170L/246K/311G/336S/340W/357E/388R/394K/401R/404L/514S/609E/659R/660W/664P/782V, 32V/38G/56T/138R/150Y/170L/246K/311G/336S/340W/357E/388K/394K/401R/404L/514S/609E/659R/660W/664P/693R/782V, 32V/38G/56T/138R/150Y/170L/246K/311 G/336S/340W/357E/388K/394K/401R/404L/514S/609E/659R/660W/664P/672S/782V, 32V/38G/56T/138R/150Y/170L/246K/311 G/336S/340W/357E/388K/394K/397S/401R/404L/514S/609E/659R/660W/664P/782V, 32V/38G/56T/138R/150Y/170L/246K/311 G/336S/340W/357E/375P/388K/394K/401R/404L/514S/609E/659R/660W/664P/782V, 32V/38G/56T/138R/150Y/170L/246K/311 G/336S/340W/357E/388K/394K/401R/404L/514S/609E/659R/660W/664P/672G/782V, 32V/38G/56T/137G/138R/150Y/170L/246K/311G/336S/340W/357E/382R/388K/394K/401R/404L/514S/609E/659R/660W/664P, 32V/38G/56T/138R/150Y/170L/246K/311 G/336S/340W/357E/388K/394K/401R/404L/514S/609E/655R/659R/660 W/664P/782V, 32V/38G/56T/137G/138R/150Y/170L/246K/311G/336S/340W/357E/382R/388K/392A/394K/401R/404L/514S/609E/659R/660W/664P/782V, 32V/38G/56T/138R/150Y/170L/246K/311G/336S/340W/357E/388K/391F/394K/401R/404L/514S/609E/659R/660W/664P/782V, 32V/38G/56T/137G/138R/150Y/170L/246K/311G/336S/340W/357E/388K/394K/401R/404L/514S/609E/655M/659R/660W/664P/721T/763S/782V, 32V/38G/56T/138R/150Y/170L/246K/311G/336S/340W/357E/388K/394K/401R/404L/514S/609E/659R/660W/664P/696L/782V, 32V/38G/56T/138R/150Y/170L/246K/311G/336S/340W/357E/388K/394K/401R/404L/514S/609E/659R/660W/664P/672R/782V, 32V/38G/56T/138R/150Y/170L/246K/311G/336S/340W/357E/388K/394K/401R/404L/514S/609E/659R/660W/663L/664P/782V, 32V/38G/56T/138R/150Y/170L/246K/311G/336S/340W/357E/367T/388K/394K/401R/404L/514S/609E/659R/660W/664P/782V, 32V/38G/56T/138R/150Y/170L/246K/311G/336S/340W/357E/367F/388K/394K/401R/404L/514S/609E/659R/660W/664P/782V, 32V/38G/56T/137G/138R/150Y/170L/246K/311G/336S/340W/357E/382G/388K/394K/401R/404L/514S/609E/659R/660W/664P/782V, 32V/38G/56T/137G/138R/150Y/170L/246K/311G/336S/340W/357E/382G/388K/392R/394K/401R/404L/514S/609E/650M/655M/659R/660W/664P, 32V/38G/56T/137G/138R/150Y/170L/246K/311G/336S/340W/357E/382R/388K/394K/401R/404L/514S/609E/629R/659R/660Y/664P/672T/782V, 32V/38G/56T/138R/150Y/170L/246K/311G/336S/340W/357E/388K/391S/394K/401R/404L/514S/609E/659R/660W/664P/782V, 32V/38G/56T/138R/150Y/170L/246K/311G/336S/340W/357E/388K/394K/401R/404L/514S/609E/616A/659R/660W/664P/782V, 32V/38G/56T/138R/150Y/170L/246K/311G/336S/340W/357E/388K/394K/401R/404L/514S/609E/659R/660W/664P/690S/782V, 32V/38G/56T/138R/150Y/170L/179N/246K/311G/336S/340W/357E/388K/394K/401R/404L/514S/609E/659R/660W/664P/782V, 32V/38G/56T/137G/138R/150Y/170L/246K/311G/336S/340W/357E/388K/394K/401R/404L/514S/609E/659R/660W/664P/782V, 32V/38G/56T/138R/150Y/170L/246K/311G/336S/340W/357P/382R/388K/394K/401R/404L/514S/609E/659R/660W/664P/670R/782V, 32V/38G/56T/138R/150Y/170L/246K/311G/336S/340W/357E/388R/394K/401R/404L/514S/609E/659R/660W/664 P/670R/672P/782V, 32V/38G/56T/138R/150Y/170L/246K/311G/336S/340W/357P/375P/388K/394K/401R/404L/514S/609E/659R/660W/664P/782V, 32V/38G/56T/138R/150Y/170L/246K/311G/336S/340W/357P/388K/394K/401R/404L/514S/609E/659R/660W/664P/672G/782V, 32V/38G/56T/138R/150Y/170L/246K/311G/336S/340W/357E/388K/394K/401R/404L/514S/609E/629K/659R/660W/664P/782V, or 32V/38G/56T/138R/150Y/170L/246K/311G/336S/340W/357E/388K/394K/401R/404L/514S/609E/655R/659R/660W/664P/670R/782V, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set at amino acid positions 32/38/56/137/138/150/170/246/311/336/340/357/388/394/401/404/514/609/629/659/660/664/782, 32/38/56/138/150/170/246/336/340/357/382/388/394/401/404/514/609/629/659/660/664/782, 32/38/56/137/138/143/150/170/246/311/336/340/357/388/394/401/404/514/609/629/659/660/664/721/782, 32/38/56/137/138/150/170/246/311/336/340/357/388/392/394/401/404/514/609/629/659/660/664/782, 32/38/137/138/143/150/170/246/311/336/340/357/388/394/401/514/609/629/659/660/664/720/782, 32/38/56/138/143/150/170/246/311/336/340/357/388/394/401/404/514/609/629/659/660/664/782, 32/38/56/138/150/170/246/311/336/340/357/388/392/394/401/404/514/609/629/659/660/664/782, 32/38/56/138/150/170/246/311/336/340/357/388/392/394/401/514/609/629/659/660/664/782, or 32/38/56/138/150/170/196/246/311/336/340/357/388/392/394/401/404/514/609/629/659/660/664/782, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution set or amino acid residues 32V/38G/56T/137G/138R/150Y/170L/246K/311G/336S/340W/357E/388K/394K/401R/404L/514S/609E/629K/659R/660W/664P/782V, 32V/38G/56E/138R/150Y/170L/246K/336S/340W/357E/382R/388K/394K/401R/404L/514S/609E/629K/659R/660W/664P/782V, 32V/38G/56E/137G/138R/143N/150Y/170L/246K/311G/336S/340W/357E/388K/394K/401R/404L/514S/609E/629K/659R/660W/664P/721A/782V, 32V/38G/56T/137G/138R/150Y/170L/246K/311G/336S/340W/357E/388K/392A/394K/401R/404L/514S/609E/629K/659R/660W/664P/782V, 32V/38G/56E/137G/138R/150Y/170L/246K/311G/336S/340W/357E/388K/392A/394K/401R/404L/514S/609E/629K/659R/660W/664P/782V, 32V/38G/137G/138R/143N/150Y/170L/246K/311G/336S/340W/357E/388K/394K/401R/514S/609E/629K/659R/660W/664P/720s/782V, 32V/38G/56E/138R/143N/150Y/170L/246R/311G/336S/340W/357E/388K/394K/401R/404L/514S/609E/629K/659R/660W/664P/782V, 32V/38G/56E/138R/150Y/170L/246K/311G/336S/340W/357E/388K/392R/394K/401R/404L/514S/609E/629K/659R/660W/664P/782V, 32V/38G/56T/138R/150Y/170L/246K/311G/336S/340W/357E/388K/392R/394K/401R/514S/609E/629K/659R/660W/664P/782V, 32V/38G/56T/138R/150Y/170L/246K/311G/336S/340W/357E/388K/392R/394K/401R/404L/514S/609E/629K/659R/660W/664P/782V, 32V/38G/56T/137G/138R/150Y/170L/246K/311G/336S/340W/357E/388K/392R/394K/401R/404L/514S/609E/629K/659R/660W/664P/782V, 32V/38G/56E/137G/138R/150Y/170L/246K/311G/336S/340W/357E/388K/394K/401R/404L/514S/609E/629K/659R/660W/664P/782V, or 32V/38G/56E/138R/150Y/170L/196S/246K/311G/336S/340W/357E/388K/392A/394K/401R/404L/514S/609E/629K/659R/660W/664P/782V, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution at an amino acid position set forth in Tables 5.1, 6.1, 7.1, 8.1, 9.1, 10.1, 11.1, 12.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least one substitution as set forth in Tables 5.1, 6.1, 7.1, 8.1, 9.1, 10.1, 11.1, 12.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set at the amino acid position(s) set forth in Tables 5.1, 6.1, 7.1, 8.1, 9.1, 10.1, 11.1, 12.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the amino acid sequence of the engineered RNA polymerase comprises at least a substitution or substitution set of an engineered RNA polymerase variant set forth in Tables 5.1, 6.1, 7.1, 8.1, 9.1, 10.1, 11.1, 12.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising at least a substitution or substitution set as set forth in Tables 5.1, 6.1, 7.1, 8.1, 9.1, 10.1, 11.1, 12.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to residues 8 to 890 of an even numbered SEQ ID NO. of SEQ ID NOs: 4-828, or to the sequence corresponding to an even numbered SEQ ID NO. of SEQ ID NOs: 4-828.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of an even-numbered SEQ ID NO. of SEQ ID NOs: 4-828, or to the reference sequence corresponding to an even-numbered SEQ ID NO. of SEQ ID NOs: 4-828, wherein the amino acid sequence comprises one or more substitutions relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the amino acid sequence of the engineered RNA polymerase comprises at least a substitution at amino acid position 13, 14, 15, 17, 19, 21, 32, 33, 38, 45, 52, 54, 55, 56, 56, 57, 60, 61, 62, 63, 67, 69, 72, 75, 78, 81, 82, 83, 84, 89, 93, 95, 100, 105, 111, 115, 122, 124, 127, 131, 133, 135, 137, 138, 139, 140, 141, 143, 150, 160, 161, 162, 164, 166, 168, 169, 170, 171, 172, 173, 179, 185, 187, 193, 196, 207, 212, 217, 218, 221, 227, 237, 239, 240, 245, 246, 250, 253, 254, 257, 261, 272, 274, 275, 311, 336, 340, 342, 351, 357, 367, 375, 379, 382, 388, 391, 392, 394, 397, 401, 402, 403, 404, 419, 424, 437, 440, 494, 510, 514, 517, 527, 529, 530, 531, 533, 536, 589, 594, 598, 607, 609, 615, 616, 617, 625, 629, 648, 650, 655, 659, 660, 663, 664, 666, 670, 672, 690, 693, 696, 718, 720, 721, 749, 759, 763, 773, 775, 779, 782, 782, 810, 854, 857, 867, 875, or 878 or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the amino acid sequence of the engineered RNA polymerase comprises at least a substitution or an amino acid residue 13N, 14Q/Y, 15T, 17N, 19E, 21G, 32S/T/V, 33E, 38G/R, 45K, 52T, 54H, 55D/S/W, 56A/E/G/N/T/V, 57G, 60G/L/M/R, 61W/Y, 62T, 63S, 67R, 69S, 72R, 75S/Y, 78S, 81C/L, 82S, 83K, 84A, 89Q, 93Y, 95Y, 100R, 105R, 111K, 115L/V, 122I, 124F, 127G, 131T, 133C, 135G/K, 137G, 138N/R, 139R, 140R, 141A, 143N/Q/R/V, 150Q/Y, 160F/L, 161R, 162V, 164W, 166D/R/T/V, 168K/L/R/S/T/V/W, 169G/L, 170L/Q/R/S, 171E/F/L/Y, 172A/E/G/K/R/V, 173D/H/L/Y/V, 179N, 185I/L/P, 187G/H/L, 193G, 196A/C/G/S/V, 207S, 212R/Y, 217P/V, 218Q/T, 221C, 227V, 237L, 239L/R, 240M, 245C, 246K/RN, 250R, 253Y, 254G/S, 257L/V, 261M, 272N, 274G/L, 275A/Y, 311A/G, 336K/L/M/S/V, 340E/K/W, 342Q, 351L, 357E/P, 367F/P/T, 375F/P, 379A/S, 382G/L/R, 388A/K/R, 391F/S, 392A/M/R, 394K, 397S, 401R, 402T, 403V, 404I/L/S/Y, 419R/S, 424A, 437P/S, 440K, 494G, 510V, 514I/L/S, 517R, 527E, 529R, 530N, 531Q, 533S, 536E/V, 589C, 594G, 598A/K/L/R/V, 607Y, 609C/E/F/P/W, 615A, 616A, 617R, 625C, 629A/D/K/R, 648F, 650M, 655M/R, 659E/R, 660H/W/Y, 663L, 664K/P, 666M, 670Q/R, 672G/P/R/S/T, 690S, 693R, 696L, 718F/N/S/V/Y, 720A/G/I/P/S, 721A/E/M/S/T/Y, 749R, 759M, 763S, 773A, 775K/R, 779R, 782E/R, 782V, 810K, 854T, 857V, 867Q, 875C, or 878M, or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the amino acid sequence of the engineered RNA polymerase comprises at least a substitution at amino acid position 32, 38, 56, 138, 150, 170, 171, 173, 246, 311, 336, 340, 357, 388, 394, 401, 404, 437, 514, 536, 609, 629, 659, 660, 664, 779, or 782, or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the amino acid sequence of the engineered RNA polymerase comprises at least a substitution or an amino acid residue 32V, 38G, 56T, 138R/N, 150Y, 170Q, 171E, 173Y, 246K, 311A/G, 336K/S, 340K/W, 357E, 388A/K, 394K, 401R, 404L/S, 437P, 514S, 536V, 609E, 629A, 659R, 660H/W, 664P, 779R, or 782V, or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, or to the reference sequence corresponding to SEQ ID NO: 4, wherein the amino acid sequence comprises one or more substitutions relative to the reference sequence corresponding to residues 8 to 890 of SEQ ID NO: 4, or relative to the reference sequence corresponding to SEQ ID NO: 4.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 28-40, or to a reference sequence corresponding to an even-numbered SEQ ID NO. of SEQ ID NOs: 28-40, wherein the amino acid sequence comprises one or more substitutions relative to the reference sequence corresponding to residues 8 to 890 of SEQ ID NO: 4, or relative to the reference sequence corresponding to SEQ ID NO: 4.

In some embodiments, the amino acid sequence of the engineered RNA polymerase comprises at least a substitution at amino acid position(s) 311/388/404/440, 138/340, 138/311/336/340/388, 311/388/440, 138/311/340/388/404/440, 138/336/342, or 351/388, wherein the amino acid positions are relative to the reference sequence corresponding to residues 8 to 890 of SEQ ID NO: 4, or relative to the reference sequence corresponding to SEQ ID NO: 4.

In some embodiments, the amino acid sequence of the engineered RNA polymerases comprises at least a substitution set or amino acid residues 311G/388K/404Y/440K, 138R/340W, 138R/311G/336S/340W/388K, 311G/388K/440K, 138R/311G/340E/388K/404Y/440K, 138R/336M/342Q, or 351L/388K, wherein the amino acid positions are relative to the reference sequence corresponding to residues 8 to 890 of SEQ ID NO: 4, or relative to the reference sequence corresponding to SEQ ID NO: 4.

In some embodiments, the amino acid sequence of the engineered RNA polymerases comprises at least a substitution set A311G/A388K/L404Y/N440K, N138R/K340W, N138R/A311G/K336S/K340W/A388K, A311G/A388K/N440K, N138R/A311G/K340E/A388K/L404Y/N440K, N138R/K336M/L342Q, or W351L/A388K, wherein the amino acid positions are relative to the reference sequence corresponding to residues 8 to 890 of SEQ ID NO: 4, or relative to the reference sequence corresponding to SEQ ID NO: 4.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 32, or to the reference sequence corresponding to SEQ ID NO: 32, wherein the amino acid sequence comprises one or more substitutions relative to the reference sequence corresponding to residues 8 to 890 of SEQ ID NO: 32, or relative to the reference sequence corresponding to SEQ ID NO: 32.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 42-188, or to a reference sequence corresponding to an even-numbered SEQ ID NO. of SEQ ID NOs: 42-188, wherein the amino acid sequence comprises one or more substitutions relative to the reference sequence corresponding to residues 8 to 890 of SEQ ID NO: 32, or relative to the reference sequence corresponding to SEQ ID NO: 32.

In some embodiments, the amino acid sequence of the engineered RNA polymerase comprises at least a substitution or substitution set at amino acid position(s) 514, 38/514, 38/394/514, 38/394/514/664, 394/514/875, 394/514, 38/124/394/514, 171/878, 170, 173, 172, 170/629, 171, 168, 166, 166/272/274/275, 169, 207, 168/217/218, 171/530, 382, 773, 670, 379, 392, 419, 57, 164, 810, 659, 54, 172/173/514/659/660, 170/171/172/514/659, 173/514/659/660, 173/514/659/810, 170/340/514/659/660/810, 170/171/173/336/340/514/659/660, 57/171/173/514/659/660/810, 170/173/514/659/660/810, 57/171/172/336/340/514/659, 173/514, 171/172/173/514/659/660, 57/170/171/173/514/659/660/810, 57/173/514, 172/173/514/857, 57/173/340/514, 170/171/173/336/514, 171/514, 57/170/173/336/514, 170/336, 173/514/659, 57/514, or 173/810, wherein the amino acid positions are relative to the reference sequence corresponding to residues 8 to 890 of SEQ ID NO: 32, or relative to the reference sequence corresponding to SEQ ID NO: 32.

In some embodiments, the amino acid sequence of the engineered RNA polymerases comprises at least a substitution or substitution set, or amino acid residue(s) 514S, 38G/514S, 38G/394K/514S, 38G/394K/514S/664P, 394K/514S/875C, 394K/514S, 38G/124F/394K/514I, 171E/878M, 170Q, 173Y, 172V, 173L, 172E, 170S/629D, 171F, 168V, 172K, 166R, 168K, 166V/272N/274L/275Y, 170R, 169L, 207S, 169G, 168L/217P/218Q, 168W, 172R, 166D, 171L, 171Y/530N, 172A, 173H, 382R, 773A, 168R, 670R, 168T, 172G, 379A, 392M, 419R, 392R, 392A, 57G, 168S, 164W, 670Q, 810K, 382G, 659R, 54H, 419S, 172E/173Y/514S/659R/660H, 170Q/171E/172E/514S/659R, 173Y/514S/659R/660H, 173Y/514S/659R/810K, 170Q/340K/514S/659R/660H/810K, 170Q/171E/173Y/336K/340K/514S/659R/660H, 57G/171E/173Y/514S/659R/660H/810K, 170Q/173Y/514S/659R/660H/810K, 57G/171E/172K/336K/340K/514S/659R, 173Y/514S, 171E/172E/173Y/514S/659R/660H, 57G/170Q/171E/173Y/514S/659R/660H/810K, 57G/173Y/514S, 172K/173D/514S/857V, 57G/173Y/340K/514S, 170Q/171E/173Y/336K/514S, 171E/514S, 57G/170Q/173Y/336K/514S, 170Q/336K, 173Y/514S/659R, 57G/514S, or 173Y/810K, wherein the amino acid positions are relative to the reference sequence corresponding to residues 8 to 890 of SEQ ID NO: 32, or relative to the reference sequence corresponding to SEQ ID NO: 32.

In some embodiments, the amino acid sequence of the engineered RNA polymerases comprises at least a substitution or substitution set L514S, R38G/L514S, R38G/R394K/L514S, R38G/R394K/L514S/W664P, R394K/L514S/G875C, R394K/L514S, R38G/I124F/R394K/L514I, K171E/N878M, K170Q, V173Y, N172V, V173L, N172E, K170S/A629D, K171F, H168V, N172K, A166R, H168K, A166V/S272N/M274L/F275Y, K170R, F169L, A207S, F169G, H168L/I217P/H218Q, H168W, N172R, A166D, K171L, K171Y/H530N, N172A, V173H, T382R, D773A, H168R, K670R, H168T, N172G, E379A, Y392M, K419R, Y392R, Y392A, R57G, H168S, L164W, K670Q, G810K, T382G, E659R, G54H, K419S, N172E/V173Y/L514S/E659R/D660H, K170Q/K171E/N172E/L514S/E659R, V173Y/L514S/E659R/D660H, V173Y/L514S/E659R/G810K, K170Q/W340K/L514S/E659R/D660H/G810K, K170Q/K171E/V173Y/S336K/W340K/L514S/E659R/D660H, R57G/K171E/V173Y/L514S/E659R/D660H/G810K, K170Q/V173Y/L514S/E659R/D660H/G810K, R57G/K171E/N172K/S336K/W340K/L514S/E659R, V173Y/L514S, K171E/N172E/V173Y/L514S/E659R/D660H, R57G/K170Q/K171E/V173Y/L514S/E659R/D660H/G810K, R57G/V173Y/L514S, N172K/V173D/L514S/A857V, R57G/V173Y/W340K/L514S, K170Q/K171E/V173Y/S336K/L514S, K171E/L514S, R57G/K170Q/V173Y/S336K/L514S, K170Q/S336K, V173Y/L514S/E659R, R57G/L514S, or V173Y/G810K, wherein the amino acid positions are relative to the reference sequence corresponding to residues 8 to 890 of SEQ ID NO: 32, or relative to the reference sequence corresponding to SEQ ID NO: 32.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 44, or to the reference sequence corresponding to SEQ ID NO: 44, wherein the amino acid sequence comprises one or more substitutions relative to the reference sequence corresponding to residues 8 to 890 of SEQ ID NO: 44, or relative to the reference sequence corresponding to SEQ ID NO: 44.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 190-194, or to a reference sequence corresponding to an even-numbered SEQ ID NO. of SEQ ID NOs: 190-194, wherein the amino acid sequence comprises one or more substitutions relative to the reference sequence corresponding to residues 8 to 890 of SEQ ID NO: 44, or relative to the reference sequence corresponding to SEQ ID NO: 44.

In some embodiments, the amino acid sequence of the engineered RNA polymerase comprises at least a substitution or substitution set at amino acid position(s) 336, 143/336/340/782 or 143/336/340/357/514, wherein the amino acid positions are relative to the reference sequence corresponding to residues 8 to 890 of SEQ ID NO: 44, or relative to the reference sequence corresponding to SEQ ID NO: 44.

In some embodiments, the amino acid sequence of the engineered RNA polymerases comprises at least a substitution or substitution set, or amino acid residue(s) 336K, 143V/336K/340K/782V or 143V/336K/340K/357E/514L, wherein the amino acid positions are relative to the reference sequence corresponding to residues 8 to 890 of SEQ ID NO: 44, or relative to the reference sequence corresponding to SEQ ID NO: 44.

In some embodiments, the amino acid sequence of the engineered RNA polymerases comprises at least a substitution or substitution set S336K, A143V/S336K/W340K/E782V, or A143V/S336K/W340K/R357E/S514L, wherein the amino acid positions are relative to the reference sequence corresponding to residues 8 to 890 of SEQ ID NO: 44, or relative to the reference sequence corresponding to SEQ ID NO: 44.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 156, or to the reference sequence corresponding to SEQ ID NO: 156, wherein the amino acid sequence comprises one or more substitutions relative to the reference sequence corresponding to residues 8 to 890 of SEQ ID NO: 156, or relative to the reference sequence corresponding to SEQ ID NO: 156.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 196-260, or to a reference sequence corresponding to an even-numbered SEQ ID NO. of SEQ ID NOs: 196-260, wherein the amino acid sequence comprises one or more substitutions relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerase comprises at least a substitution or substitution set at amino acid position(s) 207, 419, 207/336/340, 166/169, 207/670, 166/336/340, 207/419/670, 207/336/340/382/419/670, 207/382, 670, 336/670, 340/670, 382, 127/141/207/336/382/670, 207/336/382/419/670, 419/670, 166/168/207/336/340/419/670, 166/340, or 207/336/340/670, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerases comprises at least a substitution or substitution set, or amino acid residue(s) 207S, 419R, 207S/336K/340K, 166R/169L, 207S/670R, 166R/336K/340K, 207S/419R/670R, 207S/336K/340K/382G/419R/670R, 207S/382G, 670R, 336K/670R, 340K/670R, 382G, 127G/141A/207S/336K/382G/670R, 207S/336K/382G/419R/670R, 419R/670R, 166R/168V/207S/336K/340K/419R/670R, 166V/340K, or 207S/336K/340K/670R, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerases comprises at least a substitution or substitution set A207S, K419R, A207S/S336K/W340K, A166R/F169L, A207S/K670R, A166R/S336K/W340K, A207S/K419R/K670R, A207S/S336K/W340K/T382G/K419R/K670R, A207S/T382G, K670R, S336K/K670R, W340K/K670R, T382G, K127G/V141A/A207S/S336K/T382G/K670R, A207S/S336K/T382G/K419R/K670R, K419R/K670R, A166R/H168V/A207S/S336K/W340K/K419R/K670R, A166V/W340K, or A207S/S336K/W340K/K670R, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerase comprises at least a substitution or substitution set at amino acid position(s) 38/437/779, 38/437, 38, 38/207/437/779, 517/779, 38/437/527/664, 38/207/379/437, 664/670/779, 38/779, 38/437/517, 437/779, 38/437/607, 38/379/779, or 517/670/779, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerases comprises at least a substitution or substitution set, or amino acid residue(s) 38G/437P/779R, 38G/437P, 38G, 38G/207S/437P/779R, 517R/779R, 38G/437P/527E/664K, 38G/207S/379S/437P, 664K/670R/779R, 38G/779R, 38G/437P/517R, 437P/779R, 38G/437P/607Y, 38G/379S/779R, or 517R/670R/779R, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerases comprises at least a substitution or substitution set R38G/S437P/H779R, R38G/S437P, R38G, R38G/A207S/S437P/H779R, C517R/H779R, R38G/S437P/G527E/W664K, R38G/A207S/E379S/S437P, W664K/K670R/H779R, R38G/H779R, R38G/S437P/C517R, S437P/H779R, R38G/S437P/E607Y, R38G/E379S/H779R, or C517R/K670R/H779R, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerase comprises at least a substitution set at amino acid positions 357/394/664 or 138/311/336/340/357/388/394/404/664, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerases comprises at least a substitution set or amino acid residues 357E/394K/664P or 138N/311A/336K/340K/357E/388A/394K/404S/664P, wherein the amino acid positions are relative to the reference sequence corresponding to residues 8 to 890 of SEQ ID NO: 156, or relative to the reference sequence corresponding to SEQ ID NO: 156.

In some embodiments, the amino acid sequence of the engineered RNA polymerases comprises at least a substitution set R357E/R394K/W664P or R138N/G311A/S336K/W340K/R357E/K388A/R394K/L404S/W664P, wherein the amino acid positions are relative to the reference sequence corresponding to residues 8 to 890 of SEQ ID NO: 156, or relative to the reference sequence corresponding to SEQ ID NO: 156.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 234, or to the reference sequence corresponding to SEQ ID NO: 234, wherein the amino acid sequence comprises one or more substitutions relative to the reference sequence corresponding to residues 8 to 890 of SEQ ID NO: 234, or relative to the reference sequence corresponding to SEQ ID NO: 234.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 262-384, or to a reference sequence corresponding to an even-numbered SEQ ID NO. of SEQ ID NOs: 262-384, wherein the amino acid sequence comprises one or more substitutions relative to the reference sequence corresponding to residues 8 to 890 of SEQ ID NO: 234, or relative to the reference sequence corresponding to SEQ ID NO: 234.

In some embodiments, the amino acid sequence of the engineered RNA polymerase comprises at least a substitution or substitution set at amino acid position(s) 720, 196, 609, 246, 185, 594, 212, 494, 375, 721, 72, 867, 160, 718, 187, 170/173, 615, 137/138, 854, 131, 38/185/187/437, 185/187, 437/609, 38/609, 38/185/187/437/609, 437/609/720, 185/187/609, 185/187/437, 437/721, 609/720, 185/187/246, or 185/187/437/609/720, wherein the amino acid positions are relative to the reference sequence corresponding to residues 8 to 890 of SEQ ID NO: 234, or relative to the reference sequence corresponding to SEQ ID NO: 234.

In some embodiments, the amino acid sequence of the engineered RNA polymerases comprises at least a substitution or substitution set, or amino acid residue(s) 720A, 196C, 609W, 196V, 246V, 185L, 594G, 212Y, 494G, 196A, 212R, 375F, 721T, 721A, 72R, 185I, 721S, 867Q, 721Y, 160F, 718F, 609F, 720S, 187L, 718V, 170L/173V, 609E, 718S, 609C, 609P, 721M, 615A, 187H, 196S, 721E, 718Y, 137G/138N, 187G, 720G, 720P, 854T, 720I, 160L, 131T, 38R/185L/187H/437S, 185L/187H, 185L/187L, 437S/609C, 38R/609P, 38R/185L/187H/437S/609C, 437S/609P/720A, 185L/187L/609P, 185L/187L/437S, 185L/187H/437S, 437S/609P, 437S/721S, 185L/187H/609E, 609P/720A, 185L/187L/246V, or 185L/187H/437S/609P/720A, wherein the amino acid positions are relative to the reference sequence corresponding to residues 8 to 890 of SEQ ID NO: 234, or relative to the reference sequence corresponding to SEQ ID NO: 234.

In some embodiments, the amino acid sequence of the engineered RNA polymerases comprises at least a substitution or substitution set K720A, D196C, T609W, D196V, Q246V, Y185L, I594G, H212Y, E494G, D196A, H212R, D375F, K721T, K721A, A72R, Y185I, K721S, K867Q, K721Y, R160F, K718F, T609F, K720S, K187L, K718V, K170L/Y173V, T609E, K718S, T609C, T609P, K721M, K615A, K187H, D196S, K721E, K718Y, D137G/R138N, K187G, K720G, K720P, D854T, K720I, R160L, A131T, G38R/Y185L/K187H/P437S, Y185L/K187H, Y185L/K187L, P437S/T609C, G38R/T609P, G38R/Y185L/K187H/P437S/T609C, P437S/T609P/K720A, Y185L/K187L/T609P, Y185L/K187L/P437S, Y185L/K187H/P437S, P437S/T609P, P437S/K721S, Y185L/K187H/T609E, T609P/K720A, Y185L/K187L/Q246V, or Y185L/K187H/P437S/T609P/K720A, wherein the amino acid positions are relative to the reference sequence corresponding to residues 8 to 890 of SEQ ID NO: 234, or relative to the reference sequence corresponding to SEQ ID NO: 234.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 386, or to the reference sequence corresponding to SEQ ID NO: 386, wherein the amino acid sequence comprises one or more substitutions relative to the reference sequence corresponding to residues 8 to 890 of SEQ ID NO: 386, or relative to the reference sequence corresponding to SEQ ID NO: 386.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 388-460, or to a reference sequence corresponding to an even-numbered SEQ ID NO. of SEQ ID NOs: 388-460, wherein the amino acid sequence comprises one or more substitutions relative to the reference sequence corresponding to residues 8 to 890 of SEQ ID NO: 386, or relative to the reference sequence corresponding to SEQ ID NO: 386.

In some embodiments, the amino acid sequence of the engineered RNA polymerase comprises at least a substitution or substitution set at amino acid position(s) 170/609, 38/185/187/609, 38/185/187, 38/170/173, 38/173/185/187/720, 170/185/187/609, 609, 170/185/187/609/721, 38/609, 38/170/246/609, 185/187/609/718, 38/185/187/609/718/720, 38/170/246/609/718, 720, 246/609, 38/185/187/246/720, 38/609/721, 38/721, 38/720/721, 38/170/609/718/720, 38/718, 38/185/187/609/718/721, 529/609, 38/609/718, 185/187/609/718/720, 185/187/609/718/721, 38/170/718/720/721, 170/185/187/718/720, 38/173/185/187/246/609/720, 609/718/720, 170/185/187/718/720/721, 38/170/173/246/609/718, 246/721, 38/718/720, or 38/609/718/720, wherein the amino acid positions are relative to the reference sequence corresponding to residues 8 to 890 of SEQ ID NO: 386, or relative to the reference sequence corresponding to SEQ ID NO: 386.

In some embodiments, the amino acid sequence of the engineered RNA polymerases comprises at least a substitution or substitution set, or amino acid residue(s) 170L/609P, 38G/185L/187L/609C, 38G/185L/187H, 38G/170L/173V, 38G/173V/185L/187L/720A, 170L/185L/187H/609C, 609E, 170L/185L/187H/609C/721S, 38G/609E, 170L/185L/187L/609E, 38G/170L/246V/609P, 185L/187H/609E/718S, 38G/185L/187L/609W/718N/720A, 38G/170L/246V/609C/718S, 720A, 246V/609E, 38G/185L/187L/246V/720A, 38G/609C/721S, 38G/721S, 38G/720A/721S, 38G/170L/609C/718S/720A, 38G/718S, 38G/170L/246V/609P/718S, 38G/185L/187L/609P/718S/721S, 529R/609E, 38G/609P/718S, 185L/187L/609C/718S/720A, 185L/187H/609C/718S/721S, 38G/170L/718S/720A/721S, 170L/185L/187L/718s/720A, 38G/173V/185L/187L/246V/609E/720A, 609E/718S/720A, 170L/185L/187H/718s/720A/721S, 38G/170L/173V/246V/609E/718S, 246V/721S, 38G/718S/720A, or 38G/609E/718S/720A, wherein the amino acid positions are relative to the reference sequence corresponding to residues 8 to 890 of SEQ ID NO: 386, or relative to the reference sequence corresponding to SEQ ID NO: 386.

In some embodiments, the amino acid sequence of the engineered RNA polymerases comprises at least a substitution or substitution set K170L/T609P, R38G/Y185L/K187L/T609C, R38G/Y185L/K187H, R38G/K170L/Y173V, R38G/Y173V/Y185L/K187L/K720A, K170L/Y185L/K187H/T609C, T609E, K170L/Y185L/K187H/T609C/K721S, R38G/T609E, K170L/Y185L/K187L/T609E, R38G/K170L/Q246V/T609P, Y185L/K187H/T609E/K718S, R38G/Y185L/K187L/T609W/K718N/K720A, R38G/K170L/Q246V/T609C/K718S, K720A, Q246V/T609E, R38G/Y185L/K187L/Q246V/K720A, R38G/T609C/K721S, R38G/K721S, R38G/K720A/K721S, R38G/K170L/T609C/K718S/K720A, R38G/K718S, R38G/K170L/Q246V/T609P/K718S, R38G/Y185L/K187L/T609P/K718S/K721S, Q529R/T609E, R38G/T609P/K718S, Y185L/K187L/T609C/K718S/K720A, Y185L/K187H/T609C/K718S/K721S, R38G/K170L/K718S/K720A/K721S, K170L/Y185L/K187L/K718S/K720A, R38G/Y173V/Y185L/K187L/Q246V/T609E/K720A, T609E/K718S/K720A, K170L/Y185L/K187H/K718S/K720A/K721S, R38G/K170L/Y173V/Q246V/T609E/K718S, Q246V/K721S, R38G/K718S/K720A, or R38G/T609E/K718S/K720A, wherein the amino acid positions are relative to the reference sequence corresponding to residues 8 to 890 of SEQ ID NO: 386, or relative to the reference sequence corresponding to SEQ ID NO: 386.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 238, or to the reference sequence corresponding to SEQ ID NO: 238, wherein the amino acid sequence comprises one or more substitutions relative to the reference sequence corresponding to residues 8 to 890 of SEQ ID NO: 238, or relative to the reference sequence corresponding to SEQ ID NO: 238.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of an even-numbered SEQ ID NO. of SEQ ID NOs: 462-584, or to the reference sequence corresponding to an even-numbered SEQ ID NO. of SEQ ID NOs: 462-584, wherein the amino acid sequence comprises one or more substitutions relative to the reference sequence corresponding to residues 8 to 890 of SEQ ID NO: 238, or relative to the reference sequence corresponding to SEQ ID NO: 238.

In some embodiments, the amino acid sequence of the engineered RNA polymerase comprises at least a substitution or substitution set at amino acid position(s) 143, 105, 775/779, 135, 100, 246, 779/782, 749, 237, 162, 83, 218, 227, 115, 274, 81, 122, 61, 32, 139, 75, 239, 257, 240, 69/161, 253, 14, 221, 84, 133/150, 60, 275, 89, 15, 254, 625, 14/67, 21, 93, 250, 217, 19, 95, 759, 62, or 140, wherein the amino acid positions are relative to the reference sequence corresponding to residues 8 to 890 of SEQ ID NO: 238, or relative to the reference sequence corresponding to SEQ ID NO: 238.

In some embodiments, the amino acid sequence of the engineered RNA polymerases comprises at least a substitution or substitution set, or amino acid residue(s) 143N, 143Q, 143R, 105R, 775R/779R, 135K, 100R, 775K/779R, 246K, 779R/782R, 749R, 237L, 162V, 83K, 218T, 227V, 115L, 274G, 81C, 122I, 61Y, 32S, 139R, 75Y, 239R, 257L, 240M, 69S/161R, 253Y, 14Y, 221C, 84A, 115V, 133C/150Q, 60L, 60G, 32V, 275A, 89Q, 257V, 274L, 15T, 254S, 61W, 625C, 14Q/67R, 21G, 254G, 93Y, 250R, 217V, 60M, 239L, 75S, 32T, 19E, 95Y, 81L, 60R, 759M, 62T, or 140R, wherein the amino acid positions are relative to the reference sequence corresponding to residues 8 to 890 of SEQ ID NO: 238, or relative to the reference sequence corresponding to SEQ ID NO: 238.

In some embodiments, the amino acid sequence of the engineered RNA polymerases comprises at least a substitution or substitution set A143N, A143Q, A143R, K105R, E775R/H779R, S135K, K100R, E775K/H779R, Q246K, H779R/E782R, P749R, H237L, R162V, T83K, H218T, L227V, E115L, M274G, I81C, A122I, M61Y, A32S, T139R, A75Y, Q239R, Y257L, N240M, G69S/I161R, L253Y, A14Y, V221C, L84A, E115V, L133C/R150Q, K60L, K60G, A32V, F275A, I89Q, Y257V, M274L, K15T, A254S, M61W, G625C, A14Q/K67R, I21G, A254G, N93Y, T250R, I217V, K60M, Q239L, A75S, A32T, S19E, W95Y, I81L, K60R, L759M, F62T, or T140R, wherein the amino acid positions are relative to the reference sequence corresponding to residues 8 to 890 of SEQ ID NO: 238, or relative to the reference sequence corresponding to SEQ ID NO: 238.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 394, or to the reference sequence corresponding to SEQ ID NO: 394, wherein the amino acid sequence comprises one or more substitutions relative to the reference sequence corresponding to residues 8 to 890 of SEQ ID NO: 394, or relative to the reference sequence corresponding to SEQ ID NO: 394.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 586-602, or to a reference sequence corresponding to an even-numbered SEQ ID NO. of SEQ ID NOs: 586-602, wherein the amino acid sequence comprises one or more substitutions relative to the reference sequence corresponding to residues 8 to 890 of SEQ ID NO: 394, or relative to the reference sequence corresponding to SEQ ID NO: 394.

In some embodiments, the amino acid sequence of the engineered RNA polymerase comprises at least a substitution or substitution set at amino acid position(s) 609/779, 246/609, 246, 609/775/779, 105, 143/609/775, 105/135/609/775, or 111/609, 609, wherein the amino acid positions are relative to the reference sequence corresponding to residues 8 to 890 of SEQ ID NO: 394, or relative to the reference sequence corresponding to SEQ ID NO: 394.

In some embodiments, the amino acid sequence of the engineered RNA polymerases comprises at least a substitution or substitution set, or amino acid residue(s) 609E/779R, 246K/609E, 246K, 609E/775K/779R, 105R, 143N/609E/775K, 105R/135K/609E/775K, 111K/609E, or 609E, wherein the amino acid positions are relative to the reference sequence corresponding to residues 8 to 890 of SEQ ID NO: 394, or relative to the reference sequence corresponding to SEQ ID NO: 394.

In some embodiments, the amino acid sequence of the engineered RNA polymerases comprises at least a substitution or substitution set T609E/H779R, Q246K/T609E, Q246K, T609E/E775K/H779R, K105R, A143N/T609E/E775K, K105R/S135K/T609E/E775K, Q1I 1K/T609E, or T609E, wherein the amino acid positions are relative to the reference sequence corresponding to residues 8 to 890 of SEQ ID NO: 394, or relative to the reference sequence corresponding to SEQ ID NO: 394.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 588, or to the reference sequence corresponding to SEQ ID NO: 588, wherein the amino acid sequence comprises one or more substitutions relative to the reference sequence corresponding to residues 8 to 890 of SEQ ID NO: 588, or relative to the reference sequence corresponding to SEQ ID NO: 588.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 604-680, or to a reference sequence corresponding to an even-numbered SEQ ID NO. of SEQ ID NOs: 604-680, wherein the amino acid sequence comprises one or more substitutions relative to the reference sequence corresponding to residues 8 to 890 of SEQ ID NO: 588, or relative to the reference sequence corresponding to SEQ ID NO: 588.

In some embodiments, the amino acid sequence of the engineered RNA polymerase comprises at least a substitution or substitution set at amino acid position(s) 32, 531/533, 115, 83, 32/60/61, 55, 589, 357, 617, 172, 598, 56/193, 56, 536, 52, 17, 261, 78, 629, 63, 82, 207, 666, 150, 45, 135, 13, 33, or 245, wherein the amino acid positions are relative to the reference sequence corresponding to residues 8 to 890 of SEQ ID NO: 588, or relative to the reference sequence corresponding to SEQ ID NO: 588.

In some embodiments, the amino acid sequence of the engineered RNA polymerases comprises at least a substitution or substitution set, or amino acid residue(s) 32V, 531Q/533S, 115L, 83K, 32V/60M/61W, 55D, 589C, 357P, 617R, 172G, 598A, 56G/193G, 56T, 536V, 52T, 17N, 56N, 598K, 261M, 56E, 55S, 536E, 598L, 78S, 56V, 629R, 598R, 63S, 82S, 207S, 598V, 666M, 150Y, 45K, 55W, 135G, 13N, 33E, or 245C, wherein the amino acid positions are relative to the reference sequence corresponding to residues 8 to 890 of SEQ ID NO: 588, or relative to the reference sequence corresponding to SEQ ID NO: 588.

In some embodiments, the amino acid sequence of the engineered RNA polymerases comprises at least a substitution or substitution set A32V, H531Q/L533S, E115L, T83K, A32V/K60M/M61W, E55D, L589C, E357P, K617R, N172G, D598A, A56G/V193G, A56T, N536V, E52T, D17N, A56N, D598K, I261M, A56E, E55S, N536E, D598L, K78S, A56V, A629R, D598R, E63S, T82S, A207S, D598V, I666M, R150Y, A45K, E55W, S135G, 113N, D33E, or G245C, wherein the amino acid positions are relative to the reference sequence corresponding to residues 8 to 890 of SEQ ID NO: 588, or relative to the reference sequence corresponding to SEQ ID NO: 588.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 604, or to the reference sequence corresponding to SEQ ID NO: 604, wherein the amino acid sequence comprises one or more substitutions relative to the reference sequence corresponding to residues 8 to 890 of SEQ ID NO: 604, or relative to the reference sequence corresponding to SEQ ID NO: 604.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 682-704, or to a reference sequence corresponding to an even-numbered SEQ ID NO. of SEQ ID NOs: 682-704, wherein the amino acid sequence comprises one or more substitutions relative to the reference sequence corresponding to residues 8 to 890 of SEQ ID NO: 604, or relative to the reference sequence corresponding to SEQ ID NO: 604.

In some embodiments, the amino acid sequence of the engineered RNA polymerase comprises at least a substitution or substitution set at amino acid position(s) 207/245/629, 17/56/150/598, 56/629, 536, 185, 56/150, 17/56/150/207/245/598/617/629, 56/150/536/598, 56/536, 185/666, 150, or 143/150/246/782, wherein the amino acid positions are relative to the reference sequence corresponding to residues 8 to 890 of SEQ ID NO: 604, or relative to the reference sequence corresponding to SEQ ID NO: 604.

In some embodiments, the amino acid sequence of the engineered RNA polymerases comprises at least a substitution or substitution set, or amino acid residue(s) 207S/245C/629R, 17N/56E/150Y/598A, 56E/629R, 536V, 185P, 56T/150Y, 17N/56E/150Y/207S/245C/598A/617R/629R, 56E/150Y/536V/598K, 56V/536V, 185P/666M, 150Y, or 143N/150Y/246V/782V, wherein the amino acid positions are relative to the reference sequence corresponding to residues 8 to 890 of SEQ ID NO: 604, or relative to the reference sequence corresponding to SEQ ID NO: 604.

In some embodiments, the amino acid sequence of the engineered RNA polymerases comprises at least a substitution or substitution set A207S/G245C/A629R, D17N/A56E/R150Y/D598A, A56E/A629R, N536V, Y185P, A56T/R150Y, D17N/A56E/R150Y/A207S/G245C/D598A/K617R/A629R, A56E/R150Y/N536V/D598K, A56V/N536V, Y185P/I666M, R150Y, or A143N/R150Y/K246V/E782V, wherein the amino acid positions are relative to the reference sequence corresponding to residues 8 to 890 of SEQ ID NO: 604, or relative to the reference sequence corresponding to SEQ ID NO: 604.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 686, or to the reference sequence corresponding to SEQ ID NO: 686, wherein the amino acid sequence comprises one or more substitutions relative to the reference sequence corresponding to residues 8 to 890 of SEQ ID NO: 686, or relative to the reference sequence corresponding to SEQ ID NO: 686.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 706-720, or to a reference sequence corresponding to an even-numbered SEQ ID NO. of SEQ ID NOs: 706-720, wherein the amino acid sequence comprises one or more substitutions relative to the reference sequence corresponding to residues 8 to 890 of SEQ ID NO: 686, or relative to the reference sequence corresponding to SEQ ID NO: 686.

In some embodiments, the amino acid sequence of the engineered RNA polymerase comprises at least a substitution or substitution set at amino acid position(s) 143, 246/424/648/782, 246/659/782, 56/150/629/660/782, 721/782, 782, 196/659/782, or 720/721/782, wherein the amino acid positions are relative to the reference sequence corresponding to residues 8 to 890 of SEQ ID NO: 686, or relative to the reference sequence corresponding to SEQ ID NO: 686.

In some embodiments, the amino acid sequence of the engineered RNA polymerases comprises at least a substitution or substitution set, or amino acid residue(s) 143N, 246R/424A/648F/782V, 246R/659E/782V, 56T/150Y/629A/660W/782V, 721T/782V, 782V, 196G/659E/782V, or 720S/721A/782V, wherein the amino acid positions are relative to the reference sequence corresponding to residues 8 to 890 of SEQ ID NO: 686, or relative to the reference sequence corresponding to SEQ ID NO: 686.

In some embodiments, the amino acid sequence of the engineered RNA polymerases comprises at least a substitution or substitution set A143N, K246R/P424A/S648F/E782V, K246R/R659E/E782V, E56T/R150Y/R629A/H660W/E782V, K721T/E782V, E782V, D196G/R659E/E782V, or K720S/K721A/E782V, wherein the amino acid positions are relative to the reference sequence corresponding to residues 8 to 890 of SEQ ID NO: 686, or relative to the reference sequence corresponding to SEQ ID NO: 686.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 692, or to the reference sequence corresponding to SEQ ID NO: 692, wherein the amino acid sequence comprises one or more substitutions relative to the reference sequence corresponding to residues 8 to 890 of SEQ ID NO: 692, or relative to the reference sequence corresponding to SEQ ID NO: 692.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 722-730, or to a reference sequence corresponding to an even-numbered SEQ ID NO. of SEQ ID NOs: 722-730, wherein the amino acid sequence comprises one or more substitutions relative to the reference sequence corresponding to residues 8 to 890 of SEQ ID NO: 692, or relative to the reference sequence corresponding to SEQ ID NO: 692.

In some embodiments, the amino acid sequence of the engineered RNA polymerase comprises at least a substitution or substitution set at amino acid position(s) 143/659/782, 166/171/246/311, 143/721, 782, or 143/171/659/782, wherein the amino acid positions are relative to the reference sequence corresponding to residues 8 to 890 of SEQ ID NO: 692, or relative to the reference sequence corresponding to SEQ ID NO: 692.

In some embodiments, the amino acid sequence of the engineered RNA polymerases comprises at least a substitution or substitution set, or amino acid residue(s) 143N/659E/782V, 166T/171E/246R/311A, 143N/721T, 782V, or 143N/171E/659E/782V, wherein the amino acid positions are relative to the reference sequence corresponding to residues 8 to 890 of SEQ ID NO: 692, or relative to the reference sequence corresponding to SEQ ID NO: 692.

In some embodiments, the amino acid sequence of the engineered RNA polymerases comprises at least a substitution or substitution set A143N/R659E/E782V, A166T/K171E/K246R/G311A, A143N/K721T, E782V, or A143N/K171E/R659E/E782V, wherein the amino acid positions are relative to the reference sequence corresponding to residues 8 to 890 of SEQ ID NO: 692, or relative to the reference sequence corresponding to SEQ ID NO: 692.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 712, or to the reference sequence corresponding to SEQ ID NO: 712, wherein the amino acid sequence comprises one or more substitutions relative to the reference sequence corresponding to residues 8 to 890 of SEQ ID NO: 712, or relative to the reference sequence corresponding to SEQ ID NO: 712.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 732-802, or to a reference sequence corresponding to an even-numbered SEQ ID NO. of SEQ ID NOs: 732-802, wherein the amino acid sequence comprises one or more substitutions relative to the reference sequence corresponding to residues 8 to 890 of SEQ ID NO: 712, or relative to the reference sequence corresponding to SEQ ID NO: 712.

In some embodiments, the amino acid sequence of the engineered RNA polymerase comprises at least a substitution or substitution set at amino acid position(s) 367, 382, 670, 672, 357, 388, 693, 397, 375, 137/382/782, 655, 137/382/392, 391, 137/655/721/763, 696, 663, 137/382, 137/382/392/650/655/782, 137/382/629/660/672, 616, 690, 179, 137, 357/382/670, 388/670/672, 357/375, 357/672, 629, or 655/670, wherein the amino acid positions are relative to the reference sequence corresponding to residues 8 to 890 of SEQ ID NO: 712, or relative to the reference sequence corresponding to SEQ ID NO: 712.

In some embodiments, the amino acid sequence of the engineered RNA polymerases comprises at least a substitution or substitution set, or amino acid residue(s) 367P, 382R, 670R, 382L, 672P, 357P, 388R, 693R, 672S, 397S, 375P, 672G, 137G/382R/782E, 655R, 137G/382R/392A, 391F, 137G/655M/721T/763S, 696L, 672R, 663L, 367T, 367F, 137G/382G, 137G/382G/392R/650M/655M/782E, 137G/382R/629R/660Y/672T, 391S, 616A, 690S, 179N, 137G, 357P/382R/670R, 388R/670R/672P, 357P/375P, 357P/672G, 629K, or 655R/670R, wherein the amino acid positions are relative to the reference sequence corresponding to residues 8 to 890 of SEQ ID NO: 712, or relative to the reference sequence corresponding to SEQ ID NO: 712.

In some embodiments, the amino acid sequence of the engineered RNA polymerases comprises at least a substitution or substitution set L367P, T382R, K670R, T382L, L672P, E357P, K388R, S693R, L672S, A397S, D375P, L672G, D137G/T382R/V782E, Q655R, D137G/T382R/Y392A, V391F, D137G/Q655M/K721T/R763S, V696L, L672R, Q663L, L367T, L367F, D137G/T382G, D137G/T382G/Y392R/E650M/Q655M/V782E, D137G/T382R/A629R/W660Y/L672T, V391S, V616A, E690S, K179N, D137G, E357P/T382R/K670R, K388R/K670R/L672P, E357P/D375P, E357P/L672G, A629K, or Q655R/K670R, wherein the amino acid positions are relative to the reference sequence corresponding to residues 8 to 890 of SEQ ID NO: 712, or relative to the reference sequence corresponding to SEQ ID NO: 712.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 800, or to the reference sequence corresponding to SEQ ID NO: 800, wherein the amino acid sequence comprises one or more substitutions relative to the reference sequence corresponding to residues 8 to 890 of SEQ ID NO: 800, or relative to the reference sequence corresponding to SEQ ID NO: 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 804-828, or to a reference sequence corresponding to an even-numbered SEQ ID NO. of SEQ ID NOs: 804-828, wherein the amino acid sequence comprises one or more substitutions relative to the reference sequence corresponding to residues 8 to 890 of SEQ ID NO: 800, or relative to the reference sequence corresponding to SEQ ID NO: 800.

In some embodiments, the amino acid sequence of the engineered RNA polymerase comprises at least a substitution or substitution set at amino acid position(s) 137, 56/311/382, 56/137/143/721, 137/392, 56/137/392, 56/137/143/404/720, 56/143/246, 56/392, 392/404, 392, 56/137, or 56/196/392, wherein the amino acid positions are relative to the reference sequence corresponding to residues 8 to 890 of SEQ ID NO: 800, or relative to the reference sequence corresponding to SEQ ID NO: 800.

In some embodiments, the amino acid sequence of the engineered RNA polymerases comprises at least a substitution or substitution set, or amino acid residue(s) 137G, 56E/311A/382R, 56E/137G/143N/721A, 137G/392A, 56E/137G/392A, 56A/137G/143N/404Y/720S, 56E/143N/246R, 56E/392R, 392R/404Y, 392R, 137G/392R, 56E/137G, or 56E/196S/392A, wherein the amino acid positions are relative to the reference sequence corresponding to residues 8 to 890 of SEQ ID NO: 800, or relative to the reference sequence corresponding to SEQ ID NO: 800.

In some embodiments, the amino acid sequence of the engineered RNA polymerases comprises at least a substitution or substitution set D137G, T56E/G311A/T382R, T56E/D137G/A143N/K721A, D137G/Y392A, T56E/D137G/Y392A, T56A/D137G/A143N/L404Y/K720S, T56E/A143N/K246R, T56E/Y392R, Y392R/L404Y, Y392R, D137G/Y392R, T56E/D137G, or T56E/D196S/Y392A, wherein the amino acid positions are relative to the reference sequence corresponding to residues 8 to 890 of SEQ ID NO: 800, or relative to the reference sequence corresponding to SEQ ID NO: 800.

In some embodiments, the amino acid sequence of the engineered RNA polymerase comprises at least a substitution at an amino acid position set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the amino acid sequence of the engineered RNA polymerase comprises at least a substitution as set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the amino acid sequence of the engineered RNA polymerase comprises a substitution or substitution set at amino acid position(s) set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the amino acid sequence of the engineered RNA polymerase comprises at least a substitution or substitution set of an engineered RNA polymerase set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set as set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the amino acid sequence of the engineered RNA polymerase comprises residues 8 to 890 of an amino acid sequence set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, or comprises an amino acid sequence set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to residues 8 to 890 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 276, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, or 828.

In some embodiments, the engineered RNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 276, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, or 828.

In some embodiments, the amino acid sequence of the engineered RNA polymerase comprises residues 8 to 890 of an even numbered SEQ ID NO. of SEQ ID NOs: 4-828, or comprises an even numbered SEQ ID NO. of SEQ ID NOs: 4-828. In some embodiments, the amino acid sequence optionally has 1, 2, 3, 4, 5, 6, 7, 8, 9, or up to 10 amino acid insertions, deletions, or substitutions. In some embodiments, the amino acid sequence optionally has 1, 2, 3, 4, 5, 6, 7, 8, 9, or up to 10 amino acid substitutions. In some embodiments, the amino acid sequence optionally has 1, 2, 3, 4, up to 5 amino acid insertions, deletions, or substitutions. In some embodiments, the amino acid sequence optionally has 1, 2, 3, 4, up to 5 amino acid substitutions. In some embodiments, the amino acid substitutions comprises non-conservative or conservative substitutions. In some embodiments, the amino acid substitutions comprise conservative substitutions.

In some embodiments, the engineered RNA polymerase comprises an amino acid sequence comprising residues 8 to 890 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 276, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, or 828. In some embodiments, the amino acid sequence optionally has 1, 2, 3, 4, 5, 6, 7, 8, 9, or up to 10 amino acid insertions, deletions, or substitutions. In some embodiments, the amino acid sequence optionally has 1, 2, 3, 4, 5, 6, 7, 8, 9, or up to 10 amino acid substitutions. In some embodiments, the amino acid sequence optionally has 1, 2, 3, 4, up to 5 amino acid insertions, deletions, or substitutions. In some embodiments, the amino acid sequence optionally has 1, 2, 3, 4, up to 5 amino acid substitutions. In some embodiments, the amino acid substitutions comprises non-conservative or conservative substitutions. In some embodiments, the amino acid substitutions comprise conservative substitutions.

In some embodiments, the engineered RNA polymerase comprises an amino acid sequence comprising SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 276, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, or 828. In some embodiments, the amino acid sequence optionally has 1, 2, 3, 4, 5, 6, 7, 8, 9, or up to 10 amino acid insertions, deletions, or substitutions. In some embodiments, the amino acid sequence optionally has 1, 2, 3, 4, 5, 6, 7, 8, 9, or up to 10 amino acid substitutions. In some embodiments, the amino acid sequence optionally has 1, 2, 3, 4, up to 5 amino acid insertions, deletions, or substitutions. In some embodiments, the amino acid sequence optionally has 1, 2, 3, 4, up to 5 amino acid substitutions. In some embodiments, the amino acid substitutions comprises non-conservative or conservative substitutions. In some embodiments, the amino acid substitutions comprise conservative substitutions.

In some embodiments, the engineered RNA polymerase of the present disclosure has RNA polymerase activity. In some embodiments, the engineered RNA polymerase has RNA polymerase activity and at least one improved or enhanced properties as compared to a reference RNA polymerase.

In some embodiments, the engineered RNA polymerase has increased RNA polymerase activity compared to the reference RNA polymerase. In some embodiments, the engineered RNA polymerase 1.1, 1.2, 1.3, 1.4, 1.5, 2, 2.5, 3, 4, 5, 6, 7, 8, 9, 10, 15, 20, 25, 30, 35 or 40 fold, or more increased activity as compared to the reference RNA polymerase. In some embodiments, the increased activity is described as increased processivity.

In some embodiments, the engineered RNA polymerase has increased thermostability as compared to the reference RNA polymerase. In some embodiments, the engineered RNA polymerase has increased thermostability at temperatures 33° C. or greater as compared to the reference RNA polymerase.

In some embodiments, the engineered RNA polymerase has increased product integrity as compared to the reference RNA polymerase. In some embodiments, product integrity is the percent of desired RNA product over the total RNA produced in a transcription reaction, e.g., in vitro transcription reaction. In reference to the methods in the Examples, product integrity is the area of the desired full-length RNA electrophoretic peak divided by the total area of the electropherogram trace through Fragment Analyzer. In some embodiments, an increase in product integrity is characterized by increase in purity of the full length RNA product made in an in vitro transcription reaction as compared to that of a reference in vitro transcription reaction, e.g., under different conditions and/or different RNA polymerase. In some embodiments, the engineered RNA polymerase has at least 1.1, 1.2, 1.3, 1.4, 1.5, 2, 2.5, 3, 3.5, or 4 fold increased in product integrity as compared to the reference RNA polymerase.

In some embodiments, the reference RNA polymerase has the sequence corresponding to residues 8 to 890 of SEQ ID NO: 2, 4, 32, 44, 156, 234, 238, 386, 394, 588, 604, 686, 692, 712, or 800, or the sequence corresponding to SEQ ID NO: 2, 4, 32, 44, 156, 234, 238, 386, 394, 588, 604, 686, 692, 712, or 800. In some embodiments, the reference RNA polymerase has the sequence corresponding to residues 8 to 890 of SEQ ID NO: 2, or the sequence corresponding to SEQ ID NO: 2.

In some embodiments, the engineered RNA polymerase has an improved property selected from i) increased activity, ii) increased thermostability, iii) increased processivity, or iv) increased RNA product integrity, or any combination of i), ii), iii) and iv), as compared to a reference RNA polymerase. In some embodiments, the reference RNA polymerase has the sequence corresponding to residues 8 to 890 of SEQ ID NO: 2, 4, 32, 44, 156, 234, 238, 386, 394, 588, 604, 686, 692, 712, or 800, or the sequence corresponding to SEQ ID NO: 2, 4, 32, 44, 156, 234, 238, 386, 394, 588, 604, 686, 692, 712, or 800. In some embodiments, the reference RNA polymerase has the sequence corresponding to residues 8 to 890 of SEQ ID NO: 2, or the sequence corresponding to SEQ ID NO: 2.

In some embodiments, the engineered RNA polymerase is expressed as a fusion protein. In some embodiments, the engineered RNA polymerase described herein can be fused to a variety of polypeptide sequences, such as, by way of example and not limitation, polypeptide tags that can be used for detection and/or purification. In some embodiments, the fusion protein of the engineered RNA polymerase comprises a glycine-histidine or histidine-tag (His-tag). In some embodiments, the engineered RNA polymerase comprises a polylysine, e.g., 2-12 lysine units, such as for conjugation to a support medium. In some embodiments, the fusion protein of the engineered RNA polymerase comprises an epitope tag, such as c-myc, FLAG, V5, or hemagglutinin (HA). In some embodiments, the fusion protein of the engineered RNA polymerase comprises a GST, SUMO, Strep, MBP, or GFP tag. In some embodiments, the fusion is to the amino (N-) terminus of engineered RNA polymerase polypeptide. In some embodiments, the fusion is to the carboxy (C-) terminus of the engineered RNA polymerase polypeptide.

In some embodiments, the engineered RNA polymerase polypeptide described herein is an isolated composition. In some embodiments, the engineered RNA polymerase polypeptide is substantially purified, or purified as further discussed herein.

In some embodiments, the present disclosure further provides functional fragments or biologically active fragments of engineered RNA polymerase polypeptides described herein. Thus, for each and every embodiment herein of an engineered RNA polymerase, a functional fragment or biologically active fragment of the engineered RNA polymerase is provided herewith (e.g., an engineered RNA polymerase provided in Tables 5.1, 6.1, 7.1, 8.1, 9.1, 10.1, 11.1, 12.1, 13.1, 14.1, 15.1, 16.1, 17.1, 18.1, 19.1, 23.1, and 24.1). In some embodiments, a functional fragment or biologically active fragments of an engineered RNA polymerase comprises at least 90%, at least 91%, at least 92%, at least 93%, at least 94%, at least 95%, at least 96%, at least 97%, at least 98%, or at least 99% of the activity of the RNA polymerase polypeptide from which it was derived (i.e., the parent RNA polymerase). In some embodiments, functional fragments or biologically active fragments comprise at least 70%, 75%, 80%, 81%, 82%, 83%, 84%, 85%, 90%, 91%, 92%, 93%, 94%, 95%, 96%, 97%, 98%, or 99% of the parent sequence of the RNA polymerase. In some embodiments, the functional fragment will be truncated by less than 5, less than 10, less than 15, less than 10, less than 25, less than 30, less than 35, less than 40, less than 45, less than 50 amino acids, less than 55 amino acids, less than 60 amino acids, less than 65 amino acids, or less than 70 amino acids.

In some embodiments, the functional fragments or biologically active fragments of the engineered RNA polymerase polypeptide described herein include at least a mutation or mutation set in the amino acid sequence of the engineered RNA polymerase described herein. Accordingly, in some embodiments, the functional fragments or biologically active fragments of the engineered RNA polymerase displays the enhanced or improved property associated with the mutation or mutation set in the parent RNA polymerase.

### Polynucleotides Encoding Engineered Polypeptides, Expression Vectors and Host Cells

In another aspect, the present disclosure provides recombinant polynucleotides encoding the engineered RNA polymerases described herein. In some embodiments, the recombinant polynucleotides are operably linked to one or more heterologous regulatory sequences that control gene expression to create a recombinant polynucleotide construct capable of expressing the engineered RNA polymerase. In some embodiments, an expression construct containing at least one heterologous polynucleotide encoding the engineered RNA polymerase polypeptide(s) is introduced into appropriate host cells to express the corresponding RNA polymerase polypeptide(s).

As will be apparent to the skilled artisan, the availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode an engineered RNA polymerase of the present disclosure. Thus, the present disclosure provides methods and compositions for the production of each and every possible variation of polynucleotides that could be made that encode the engineered RNA polymerase polypeptides described herein by selecting combinations based on the possible codon choices, and all such polynucleotide variants are to be considered specifically disclosed for any polypeptide described herein, including the amino acid sequences presented in the Examples (e.g., Tables 5.1, 6.1, 7.1, 8.1, 9.1, 10.1, 11.1, 12.1, 13.1, 14.1, 15.1, 16.1, 17.1, 18.1, 19.1, 23.1, and 24.1) and in the Sequence Listing.

In some embodiments, the codons are preferably optimized for utilization by the chosen host cell for protein production. In some embodiments, preferred codons in bacteria are used for expression in bacteria. In some embodiments, preferred codons in fungal cells are used for expression in fungal cells. In some embodiments, preferred codons in insect cells are used for expression in insect cells. In some embodiments, preferred codons in mammalian cells are used for expression in mammalian cells. In some embodiments, codon optimized polynucleotides encoding an engineered RNA polymerase polypeptide described herein contain preferred codons at about 40%, 50%, 60%, 70%, 80%, 90%, or greater than 90% of the codon positions in the full-length coding region.

Accordingly, in some embodiments, a recombinant polynucleotide of the present disclosure comprises a polynucleotide sequence encoding an engineered RNA polymerase polypeptide described herein. In some embodiments, the polynucleotide sequence of the recombinant polynucleotide is codon optimized. In some embodiments, the polynucleotide sequence of the recombinant polynucleotide is codon optimized for expression in bacterial cells.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of SEQ ID NO: 2, 4, 32, 44, 156, 234, 238, 386, 394, 588, 604, 686, 692, 712, or 800, or to a reference sequence corresponding to SEQ ID NO: 2, 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 2, or to the reference sequence corresponding to SEQ ID NO: 2, wherein the amino acid sequence comprises one or more substitutions relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to residues 8 to 890 of an even numbered SEQ ID NO. of SEQ ID NOs: 4-828, or to the sequence corresponding to an even numbered SEQ ID NO. of SEQ ID NOs: 4-828, wherein the amino acid sequence comprises one or more substitutions relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding an engineered RNA polymerase comprising an amino acid sequence comprising at least a substitution at amino acid position 13, 14, 15, 17, 19, 21, 32, 33, 38, 45, 52, 54, 55, 56, 56, 57, 60, 61, 62, 63, 67, 69, 72, 75, 78, 81, 82, 83, 84, 89, 93, 95, 100, 105, 111, 115, 122, 124, 127, 131, 133, 135, 137, 138, 139, 140, 141, 143, 150, 160, 161, 162, 164, 166, 168, 169, 170, 171, 172, 173, 179, 185, 187, 193, 196, 207, 212, 217, 218, 221, 227, 237, 239, 240, 245, 246, 250, 253, 254, 257, 261, 272, 274, 275, 311, 336, 340, 342, 351, 357, 367, 375, 379, 382, 388, 391, 392, 394, 397, 401, 402, 403, 404, 419, 424, 437, 440, 494, 510, 514, 517, 527, 529, 530, 531, 533, 536, 589, 594, 598, 607, 609, 615, 616, 617, 625, 629, 648, 650, 655, 659, 660, 663, 664, 666, 670, 672, 690, 693, 696, 718, 720, 721, 749, 759, 763, 773, 775, 779, 782, 782, 810, 854, 857, 867, 875, or 878, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding the engineered RNA polymerase comprising an amino acid sequence comprising at least a substitution at amino acid position 32, 38, 56, 138, 150, 170, 171, 173, 246, 311, 336, 340, 357, 388, 394, 401, 404, 437, 514, 536, 609, 629, 659, 660, 664, 779, or 782, or combinations thereof, wherein the amino acid positions are relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding the engineered RNA polymerase comprising an amino acid sequence comprising at least a substitution or substitution set at amino acid position(s) 401/404, 336, 138, 401/403/404, 336/510, 388, 340, 401/402/404, or 351/357, wherein the amino acid positions are relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding the RNA polymerase comprising an amino acid sequence comprising at an amino acid position set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding the RNA polymerase comprising an amino acid sequence comprising at least a substitution set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding the RNA polymerase comprising an amino acid sequence comprising at least a substitution or substitution set at amino acid position(s) set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding an RNA polymerase comprising an amino acid sequence comprising at least a substitution or substitution set of an engineered RNA polymerase set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, or relative to the reference sequence corresponding to SEQ ID NO: 2.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800.

In some embodiments, the recombinant polynucleotide comprising a polynucleotide sequence encoding the engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to residues 8 to 890 of an even numbered SEQ ID NO. of SEQ ID NOs: 4-828, or to the sequence corresponding to an even numbered SEQ ID NO. of SEQ ID NOs: 4-828.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of an even-numbered SEQ ID NO. of SEQ ID NOs: 4-828, or to the reference sequence corresponding to an even-numbered SEQ ID NO. of SEQ ID NOs: 4-828, wherein the amino acid sequence comprises one or more substitutions relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the recombinant polynucleotide comprises a polynucleotide sequence encoding the engineered RNA polymerase comprising an amino acid sequence comprising at least a substitution at amino acid position 13, 14, 15, 17, 19, 21, 32, 33, 38, 45, 52, 54, 55, 56, 56, 57, 60, 61, 62, 63, 67, 69, 72, 75, 78, 81, 82, 83, 84, 89, 93, 95, 100, 105, 111, 115, 122, 124, 127, 131, 133, 135, 137, 138, 139, 140, 141, 143, 150, 160, 161, 162, 164, 166, 168, 169, 170, 171, 172, 173, 179, 185, 187, 193, 196, 207, 212, 217, 218, 221, 227, 237, 239, 240, 245, 246, 250, 253, 254, 257, 261, 272, 274, 275, 311, 336, 340, 342, 351, 357, 367, 375, 379, 382, 388, 391, 392, 394, 397, 401, 402, 403, 404, 419, 424, 437, 440, 494, 510, 514, 517, 527, 529, 530, 531, 533, 536, 589, 594, 598, 607, 609, 615, 616, 617, 625, 629, 648, 650, 655, 659, 660, 663, 664, 666, 670, 672, 690, 693, 696, 718, 720, 721, 749, 759, 763, 773, 775, 779, 782, 782, 810, 854, 857, 867, 875, or 878, or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the recombinant polynucleotide comprises a polynucleotide sequence encoding an engineered RNA polymerase comprising an amino acid sequence comprising at least a substitution at amino acid position 32, 38, 56, 138, 150, 170, 171, 173, 246, 311, 336, 340, 357, 388, 394, 401, 404, 437, 514, 536, 609, 629, 659, 660, 664, 779, or 782, or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, or to the reference sequence corresponding to SEQ ID NO: 4, wherein the amino acid sequence comprises one or more substitutions relative to the reference sequence corresponding to residues 8 to 890 of SEQ ID NO: 4, or relative to the reference sequence corresponding to SEQ ID NO: 4.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 28-40, or to a reference sequence corresponding to an even-numbered SEQ ID NO. of SEQ ID NOs: 28-40, wherein the amino acid sequence comprises one or more substitutions relative to the reference sequence corresponding to residues 8 to 890 of SEQ ID NO: 4, or relative to the reference sequence corresponding to SEQ ID NO: 4.

In some embodiments, the recombinant polynucleotide comprises a polynucleotide sequence encoding an engineered RNA polymerase comprising an amino acid sequence comprising at least a substitution set at amino acid position(s) 311/388/404/440, 138/340, 138/311/336/340/388, 311/388/440, 138/311/340/388/404/440, 138/336/342, or 351/388, wherein the amino acid positions are relative to the reference sequence corresponding to residues 8 to 890 of SEQ ID NO: 4, or relative to the reference sequence corresponding to SEQ ID NO: 4.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 32, or to the reference sequence corresponding to SEQ ID NO: 32, wherein the amino acid sequence comprises one or more substitutions relative to the reference sequence corresponding to residues 8 to 890 of SEQ ID NO: 32, or relative to the reference sequence corresponding to SEQ ID NO: 32.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 42-188, or to a reference sequence corresponding to an even-numbered SEQ ID NO. of SEQ ID NOs: 42-188, wherein the amino acid sequence comprises one or more substitutions relative to the reference sequence corresponding to residues 8 to 890 of SEQ ID NO: 32, or relative to the reference sequence corresponding to SEQ ID NO: 32.

In some embodiments, the recombinant polynucleotide comprises a polynucleotide sequence encoding an engineered RNA polymerase comprising an amino acid sequence comprising at least a substitution or substitution set at amino acid position(s) 514, 38/514, 38/394/514, 38/394/514/664, 394/514/875, 394/514, 38/124/394/514, 171/878, 170, 173, 172, 170/629, 171, 168, 166, 166/272/274/275, 169, 207, 168/217/218, 171/530, 382, 773, 670, 379, 392, 419, 57, 164, 810, 659, 54, 172/173/514/659/660, 170/171/172/514/659, 173/514/659/660, 173/514/659/810, 170/340/514/659/660/810, 170/171/173/336/340/514/659/660, 57/171/173/514/659/660/810, 170/173/514/659/660/810, 57/171/172/336/340/514/659, 173/514, 171/172/173/514/659/660, 57/170/171/173/514/659/660/810, 57/173/514, 172/173/514/857, 57/173/340/514, 170/171/173/336/514, 171/514, 57/170/173/336/514, 170/336, 173/514/659, 57/514, or 173/810, wherein the amino acid positions are relative to the reference sequence corresponding to residues 8 to 890 of SEQ ID NO: 32, or relative to the reference sequence corresponding to SEQ ID NO: 32.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 44, or to the reference sequence corresponding to SEQ ID NO: 44, wherein the amino acid sequence comprises one or more substitutions relative to the reference sequence corresponding to residues 8 to 890 of SEQ ID NO: 44, or relative to the reference sequence corresponding to SEQ ID NO: 44.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 190-194, or to a reference sequence corresponding to an even-numbered SEQ ID NO. of SEQ ID NOs: 190-194, wherein the amino acid sequence comprises one or more substitutions relative to the reference sequence corresponding to residues 8 to 890 of SEQ ID NO: 44, or relative to the reference sequence corresponding to SEQ ID NO: 44.

In some embodiments, the recombinant polynucleotide comprises a polynucleotide sequence encoding an engineered RNA polymerase comprising an amino acid sequence comprising at least a substitution or substitution set at amino acid position(s) 336, 143/336/340/782, or 143/336/340/357/514, wherein the amino acid positions are relative to the reference sequence corresponding to residues 8 to 890 of SEQ ID NO: 44, or relative to the reference sequence corresponding to SEQ ID NO: 44.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 156, or to the reference sequence corresponding to SEQ ID NO: 156, wherein the amino acid sequence comprises one or more substitutions relative to the reference sequence corresponding to residues 8 to 890 of SEQ ID NO: 156, or relative to the reference sequence corresponding to SEQ ID NO: 156.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 196-260, or to a reference sequence corresponding to an even-numbered SEQ ID NO. of SEQ ID NOs: 196-260, wherein the amino acid sequence comprises one or more substitutions relative to the reference sequence corresponding to residues 8 to 890 of SEQ ID NO: 156, or relative to the reference sequence corresponding to SEQ ID NO: 156.

In some embodiments, the recombinant polynucleotide comprises a polynucleotide sequence encoding an engineered RNA polymerase comprising an amino acid sequence comprising at least a substitution or substitution set at amino acid position(s) 207, 419, 207/336/340, 166/169, 207/670, 166/336/340, 207/419/670, 207/336/340/382/419/670, 207/382, 670, 336/670, 340/670, 382, 127/141/207/336/382/670, 207/336/382/419/670, 419/670, 166/168/207/336/340/419/670, 166/340, or 207/336/340/670, wherein the amino acid positions are relative to the reference sequence corresponding to residues 8 to 890 of SEQ ID NO: 156, or relative to the reference sequence corresponding to SEQ ID NO: 156.

In some embodiments, the recombinant polynucleotide comprises a polynucleotide sequence encoding an engineered RNA polymerase comprising an amino acid sequence comprising at least a substitution or substitution set at amino acid position(s) 38/437/779, 38/437, 38, 38/207/437/779, 517/779, 38/437/527/664, 38/207/379/437, 664/670/779, 38/779, 38/437/517, 437/779, 38/437/607, 38/379/779, or 517/670/779, wherein the amino acid positions are relative to the reference sequence corresponding to residues 8 to 890 of SEQ ID NO: 156, or relative to the reference sequence corresponding to SEQ ID NO: 156.

In some embodiments, the recombinant polynucleotide comprises a polynucleotide sequence encoding an engineered RNA polymerase comprising an amino acid sequence comprising at least a substitution set at amino acid position(s) 357/394/664 or 138/311/336/340/357/388/394/404/664, wherein the amino acid positions are relative to the reference sequence corresponding to residues 8 to 890 of SEQ ID NO: 156, or relative to the reference sequence corresponding to SEQ ID NO: 156.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 234, or to the reference sequence corresponding to SEQ ID NO: 234, wherein the amino acid sequence comprises one or more substitutions relative to the reference sequence corresponding to residues 8 to 890 of SEQ ID NO: 234, or relative to the reference sequence corresponding to SEQ ID NO: 234.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 262-384, or to a reference sequence corresponding to an even-numbered SEQ ID NO. of SEQ ID NOs: 262-384, wherein the amino acid sequence comprises one or more substitutions relative to the reference sequence corresponding to residues 8 to 890 of SEQ ID NO: 234, or relative to the reference sequence corresponding to SEQ ID NO: 234.

In some embodiments, the recombinant polynucleotide comprises a polynucleotide sequence encoding an engineered RNA polymerase comprising an amino acid sequence comprising at least a substitution or substitution set at amino acid position(s) 720, 196, 609, 246, 185, 594, 212, 494, 375, 721, 72, 867, 160, 718, 187, 170/173, 615, 137/138, 854, 131, 38/185/187/437, 185/187, 437/609, 38/609, 38/185/187/437/609, 437/609/720, 185/187/609, 185/187/437, 437/721, 609/720, 185/187/246, or 185/187/437/609/720, wherein the amino acid positions are relative to the reference sequence corresponding to residues 8 to 890 of SEQ ID NO: 234, or relative to the reference sequence corresponding to SEQ ID NO: 234.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 386, or to the reference sequence corresponding to SEQ ID NO: 386, wherein the amino acid sequence comprises one or more substitutions relative to the reference sequence corresponding to residues 8 to 890 of SEQ ID NO: 386, or relative to the reference sequence corresponding to SEQ ID NO: 386.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 388-460, or to a reference sequence corresponding to an even-numbered SEQ ID NO. of SEQ ID NOs: 388-460, wherein the amino acid sequence comprises one or more substitutions relative to the reference sequence corresponding to residues 8 to 890 of SEQ ID NO: 386, or relative to the reference sequence corresponding to SEQ ID NO: 386.

In some embodiments, the recombinant polynucleotide comprises a polynucleotide sequence encoding an engineered RNA polymerase comprising an amino acid sequence comprising at least a substitution or substitution set at amino acid position(s) 170/609, 38/185/187/609, 38/185/187, 38/170/173, 38/173/185/187/720, 170/185/187/609, 609, 170/185/187/609/721, 38/609, 38/170/246/609, 185/187/609/718, 38/185/187/609/718/720, 38/170/246/609/718, 720, 246/609, 38/185/187/246/720, 38/609/721, 38/721, 38/720/721, 38/170/609/718/720, 38/718, 38/185/187/609/718/721, 529/609, 38/609/718, 185/187/609/718/720, 185/187/609/718/721, 38/170/718/720/721, 170/185/187/718/720, 38/173/185/187/246/609/720, 609/718/720, 170/185/187/718/720/721, 38/170/173/246/609/718, 246/721, 38/718/720, or 38/609/718/720, wherein the amino acid positions are relative to the reference sequence corresponding to residues 8 to 890 of SEQ ID NO: 386, or relative to the reference sequence corresponding to SEQ ID NO: 386.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 238, or to the reference sequence corresponding to SEQ ID NO: 238, wherein the amino acid sequence comprises one or more substitutions relative to the reference sequence corresponding to residues 8 to 890 of SEQ ID NO: 238, or relative to the reference sequence corresponding to SEQ ID NO: 238.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of an even-numbered SEQ ID NO. of SEQ ID NOs: 462-584, or to the reference sequence corresponding to an even-numbered SEQ ID NO. of SEQ ID NOs: 462-584, wherein the amino acid sequence comprises one or more substitutions relative to the reference sequence corresponding to residues 8 to 890 of SEQ ID NO: 238, or relative to the reference sequence corresponding to SEQ ID NO: 238.

In some embodiments, the recombinant polynucleotide comprises a polynucleotide sequence encoding an engineered RNA polymerase comprising an amino acid sequence comprising at least a substitution or substitution set at amino acid position(s) 143, 105, 775/779, 135, 100, 246, 779/782, 749, 237, 162, 83, 218, 227, 115, 274, 81, 122, 61, 32, 139, 75, 239, 257, 240, 69/161, 253, 14, 221, 84, 133/150, 60, 275, 89, 15, 254, 625, 14/67, 21, 93, 250, 217, 19, 95, 759, 62, or 140, wherein the amino acid positions are relative to the reference sequence corresponding to residues 8 to 890 of SEQ ID NO: 238, or relative to the reference sequence corresponding to SEQ ID NO: 238.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 394, or to the reference sequence corresponding to SEQ ID NO: 394, wherein the amino acid sequence comprises one or more substitutions relative to the reference sequence corresponding to residues 8 to 890 of SEQ ID NO: 394, or relative to the reference sequence corresponding to SEQ ID NO: 394.

In some embodiments, the recombinant polynucleotide comprises a polynucleotide sequence encoding the engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 586-602, or to a reference sequence corresponding to an even-numbered SEQ ID NO. of SEQ ID NOs: 586-602, wherein the amino acid sequence comprises one or more substitutions relative to the reference sequence corresponding to residues 8 to 890 of SEQ ID NO: 394, or relative to the reference sequence corresponding to SEQ ID NO: 394.

In some embodiments, the recombinant polynucleotide comprises a polynucleotide sequence encoding an engineered RNA polymerase comprising an amino acid sequence comprising at least a substitution or substitution set at amino acid position(s) 609/779, 246/609, 246, 609/775/779, 105, 143/609/775, 105/135/609/775, or 111/609, 609, wherein the amino acid positions are relative to the reference sequence corresponding to residues 8 to 890 of SEQ ID NO: 394, or relative to the reference sequence corresponding to SEQ ID NO: 394.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 588, or to the reference sequence corresponding to SEQ ID NO: 588, wherein the amino acid sequence comprises one or more substitutions relative to the reference sequence corresponding to residues 8 to 890 of SEQ ID NO: 588, or relative to the reference sequence corresponding to SEQ ID NO: 588.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 604-680, or to a reference sequence corresponding to an even-numbered SEQ ID NO. of SEQ ID NOs: 604-680, wherein the amino acid sequence comprises one or more substitutions relative to the reference sequence corresponding to residues 8 to 890 of SEQ ID NO: 588, or relative to the reference sequence corresponding to SEQ ID NO: 588.

In some embodiments, the recombinant polynucleotide comprises a polynucleotide sequence encoding an engineered RNA polymerase comprising an amino acid sequence comprising at least a substitution or substitution set at amino acid position(s) 32, 531/533, 115, 83, 32/60/61, 55, 589, 357, 617, 172, 598, 56/193, 56, 536, 52, 17, 261, 78, 629, 63, 82, 207, 666, 150, 45, 135, 13, 33, or 245, wherein the amino acid positions are relative to the reference sequence corresponding to residues 8 to 890 of SEQ ID NO: 588, or relative to the reference sequence corresponding to SEQ ID NO: 588.

In some embodiments, the recombinant polynucleotide comprises a polynucleotide sequence encoding the engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 604, or to the reference sequence corresponding to SEQ ID NO: 604, wherein the amino acid sequence comprises one or more substitutions relative to the reference sequence corresponding to residues 8 to 890 of SEQ ID NO: 604, or relative to the reference sequence corresponding to SEQ ID NO: 604.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 682-704, or to a reference sequence corresponding to an even-numbered SEQ ID NO. of SEQ ID NOs: 682-704, wherein the amino acid sequence comprises one or more substitutions relative to the reference sequence corresponding to residues 8 to 890 of SEQ ID NO: 604, or relative to the reference sequence corresponding to SEQ ID NO: 604.

In some embodiments, the recombinant polynucleotide comprises a polynucleotide sequence encoding an engineered RNA polymerase comprising an amino acid sequence comprising at least a substitution or substitution set at amino acid position(s) 207/245/629, 17/56/150/598, 56/629, 536, 185, 56/150, 17/56/150/207/245/598/617/629, 56/150/536/598, 56/536, 185/666, 150, or 143/150/246/782, wherein the amino acid positions are relative to the reference sequence corresponding to residues 8 to 890 of SEQ ID NO: 604, or relative to the reference sequence corresponding to SEQ ID NO: 604.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 686, or to the reference sequence corresponding to SEQ ID NO: 686, wherein the amino acid sequence comprises one or more substitutions relative to the reference sequence corresponding to residues 8 to 890 of SEQ ID NO: 686, or relative to the reference sequence corresponding to SEQ ID NO: 686.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 706-720, or to a reference sequence corresponding to an even-numbered SEQ ID NO. of SEQ ID NOs: 706-720, wherein the amino acid sequence comprises one or more substitutions relative to the reference sequence corresponding to residues 8 to 890 of SEQ ID NO: 686, or relative to the reference sequence corresponding to SEQ ID NO: 686.

In some embodiments, the recombinant polynucleotide comprises a polynucleotide sequence encoding an engineered RNA polymerase comprising an amino acid sequence comprising at least a substitution or substitution set at amino acid position(s) 143, 246/424/648/782, 246/659/782, 56/150/629/660/782, 721/782, 782, 196/659/782, or 720/721/782, wherein the amino acid positions are relative to the reference sequence corresponding to residues 8 to 890 of SEQ ID NO: 686, or relative to the reference sequence corresponding to SEQ ID NO: 686.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 692, or to the reference sequence corresponding to SEQ ID NO: 692, wherein the amino acid sequence comprises one or more substitutions relative to the reference sequence corresponding to residues 8 to 890 of SEQ ID NO: 692, or relative to the reference sequence corresponding to SEQ ID NO: 692.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 722-730, or to a reference sequence corresponding to an even-numbered SEQ ID NO. of SEQ ID NOs: 722-730, wherein the amino acid sequence comprises one or more substitutions relative to the reference sequence corresponding to residues 8 to 890 of SEQ ID NO: 692, or relative to the reference sequence corresponding to SEQ ID NO: 692.

In some embodiments, the recombinant polynucleotide comprises a polynucleotide sequence encoding an engineered RNA polymerase comprising an amino acid sequence comprising at least a substitution or substitution set at amino acid position(s) 143/659/782, 166/171/246/311, 143/721, 782, or 143/171/659/782, wherein the amino acid positions are relative to the reference sequence corresponding to residues 8 to 890 of SEQ ID NO: 692, or relative to the reference sequence corresponding to SEQ ID NO: 692.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 712, or to the reference sequence corresponding to SEQ ID NO: 712, wherein the amino acid sequence comprises one or more substitutions relative to the reference sequence corresponding to residues 8 to 890 of SEQ ID NO: 712, or relative to the reference sequence corresponding to SEQ ID NO: 712.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 732-802, or to a reference sequence corresponding to an even-numbered SEQ ID NO. of SEQ ID NOs: 732-802, wherein the amino acid sequence comprises one or more substitutions relative to the reference sequence corresponding to residues 8 to 890 of SEQ ID NO: 712, or relative to the reference sequence corresponding to SEQ ID NO: 712.

In some embodiments, the recombinant polynucleotide comprises a polynucleotide sequence encoding an engineered RNA polymerase comprising an amino acid sequence comprising at least a substitution or substitution set at amino acid position(s) 367, 382, 670, 672, 357, 388, 693, 397, 375, 137/382/782, 655, 137/382/392, 391, 137/655/721/763, 696, 663, 137/382, 137/382/392/650/655/782, 137/382/629/660/672, 616, 690, 179, 137, 357/382/670, 388/670/672, 357/375, 357/672, 629, or 655/670, wherein the amino acid positions are relative to the reference sequence corresponding to residues 8 to 890 of SEQ ID NO: 712, or relative to the reference sequence corresponding to residues 8 to 890 of SEQ ID NO: 712, or relative to the reference sequence corresponding to SEQ ID NO: 712.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 800, or to the reference sequence corresponding to SEQ ID NO: 800, wherein the amino acid sequence comprises one or more substitutions relative to the reference sequence corresponding to residues 8 to 890 of SEQ ID NO: 800, or relative to the reference sequence corresponding to SEQ ID NO: 800.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of an even-numbered SEQ ID NO. of SEQ ID NOs: 804-828, or to a reference sequence corresponding to an even-numbered SEQ ID NO. of SEQ ID NOs: 804-828, wherein the amino acid sequence comprises one or more substitutions relative to the reference sequence corresponding to residues 8 to 890 of SEQ ID NO: 800, or relative to the reference sequence corresponding to SEQ ID NO: 800.

In some embodiments, the recombinant polynucleotide comprises a polynucleotide sequence encoding an engineered RNA polymerase comprising an amino acid sequence comprising at least a substitution or substitution set at amino acid position(s) 137, 56/311/382, 56/137/143/721, 137/392, 56/137/392, 56/137/143/404/720, 56/143/246, 56/392, 392/404, 392, 56/137, or 56/196/392, wherein the amino acid positions are relative to the reference sequence corresponding to residues 8 to 890 of SEQ ID NO: 800, or relative to the reference sequence corresponding to SEQ ID NO: 800.

In some embodiments, the recombinant polynucleotide comprises a polynucleotide sequence encoding an engineered RNA polymerase comprising an amino acid sequence comprising a substitution at an amino acid position set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to residues 8 to 890 of SEQ ID NO: 2, 4, 32, 44, 156, 234, 238, 386, 394, 588, 604, 686, 692, 712, or 800.

In some embodiments, the recombinant polynucleotide comprises a polynucleotide sequence encoding an engineered RNA polymerase comprising an amino acid sequence comprising at least one substitution set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to residues 8 to 890 of SEQ ID NO: 2, 4, 32, 44, 156, 234, 238, 386, 394, 588, 604, 686, 692, 712, or 800.

In some embodiments, the recombinant polynucleotide comprises a polynucleotide sequence encoding an engineered RNA polymerase comprising an amino acid sequence comprising at least a substitution or substitution set at the amino acid position(s) of an engineered RNA polymerase set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to residues 8 to 890 of SEQ ID NO: 2, 4, 32, 44, 156, 234, 238, 386, 394, 588, 604, 686, 692, 712, or 800.

In some embodiments, the recombinant polynucleotide comprises a polynucleotide sequence encoding an engineered RNA polymerase comprising an amino acid sequence comprising at least a substitution or substitution set of an engineered RNA polymerase variant set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to residues 8 to 890 of SEQ ID NO: 2, 4, 32, 44, 156, 234, 238, 386, 394, 588, 604, 686, 692, 712, or 800.

In some embodiments, the recombinant polynucleotide comprises a polynucleotide sequence encoding an engineered RNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising at least a substitution or substitution set as set forth in Tables 5.1, 6.1, 7.1, 8.1, 9.1, 10.1, 11.1, 12.1, 13.1, 14.1, 15.1, 16.1, 17.1, 18.1, 19.1, 23.1, and 24.1, wherein the amino acid positions are relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

In some embodiments, the recombinant polynucleotide comprises a polynucleotide sequence encoding the engineered RNA polymerase comprising an amino acid sequence comprising residues 8 to 890 of an even numbered SEQ ID NO. of SEQ ID NOs: 4-828, or comprising an even numbered SEQ ID NO. of SEQ ID NOs: 4-828.

In some embodiments, the recombinant polynucleotide comprises a polynucleotide sequence encoding the engineered RNA polymerase comprising an amino acid sequence comprising residues 8 to 890 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 276, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, or 828.

In some embodiments, the recombinant polynucleotide comprises a polynucleotide sequence encoding the engineered RNA polymerase comprising an amino acid sequence comprising SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 276, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, or 828.

In some embodiments, the recombinant polynucleotide comprises a polynucleotide sequence having at least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 21 to 2670 of an odd-numbered SEQ ID NO. of SEQ ID NOs: 3-827, or corresponding to an odd-numbered SEQ ID NO. of SEQ ID NOs: 3-827, wherein the recombinant polynucleotide encodes an RNA polymerase.

In some embodiments, the recombinant polynucleotide comprises a polynucleotide sequence having at least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 21 to 2670 of SEQ ID NO: 3, 31, 43, 155, 233, 237, 381, 393, 587, 603, 685, 691, 711, or 799, or corresponding to SEQ ID NO: 3, 31, 43, 155, 233, 237, 381, 393, 587, 603, 685, 691, 711, or 799, wherein the recombinant polynucleotide encodes an RNA polymerase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 21 to 2670 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 275, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, or 827, wherein the recombinant polynucleotide encodes an RNA polymerase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference polynucleotide sequence corresponding to SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 275, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, or 827, wherein the recombinant polynucleotide encodes an RNA polymerase.

In some embodiments, the recombinant polynucleotide comprises a polynucleotide sequence comprising nucleotide residues 21 to 2670 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 275, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, or 827.

In some embodiments, the engineered RNA polymerase comprises an amino acid sequence comprising SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 275, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, or 827.

In some embodiments, a recombinant polynucleotide encoding any of the RNA polymerase herein is manipulated in a variety of ways to facilitate expression of the RNA polymerase polypeptide. As such, in some embodiments, the present disclosure provides an expression vector comprising a recombinant polynucleotide encoding an engineered RNA polymerase described herein. In some embodiments, the expression vector comprises one or more control sequences operably linked to the recombinant polynucleotide to regulate expression of the RNA polymerase encoding polynucleotides and/or expression of the corresponding polypeptides. In some embodiments, the control sequence includes, among others, promoters, leader sequences, polyadenylation sequences, propeptide sequences, signal peptide sequences, and transcription terminators. In some embodiments, the control sequences are selected depending on the type of hosts into which the expression vectors are to be introduced.

In some embodiments, suitable promoters are selected based on the host cells. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (see, e.g., Villa-Kamaroff et al., Proc. Natl Acad. Sci. USA, 1978, 75:3727-3731), as well as the tac promoter (see, e.g., DeBoer et al., Proc. Natl Acad. Sci. USA, 1983, 80:21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (see, e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (see, e.g., Romanos et al., Yeast, 1992, 8:423-488). Exemplary promoters for use in insect cells include, but are not limited to, polyhedrin, p10, ELT, OpIE2, and hr5/ie1 promoters. Exemplary promoters for use in mammalian cells include, but are not limited to, those from cytomegalovirus (CMV), chicken β-actin promoter fused with the CMV enhancer, Simian virus 40 (SV40), from Homo sapiens phosphoglycerate kinase, beta actin, elongation factor-1a or glyceraldehyde-3-phosphate dehydrogenase, or from Gallus β-actin.

In some embodiments, the control sequence is a suitable transcription terminator sequence (i.e., a sequence recognized by a host cell to terminate transcription). In some embodiments, the terminator sequence is operably linked to the 3′ terminus of the nucleic acid sequence encoding the RNA polymerase polypeptide. Any suitable terminator which is functional in the host cell of choice finds use in the present invention. For bacterial expression, the transcription terminators can be a Rho-dependent terminators that rely on a Rho transcription factor, or a Rho-independent, or intrinsic terminators, which do not require a transcription factor. Exemplary bacterial transcription terminators are described in Peters et al., J Mol Biol., 2011, 412(5):793-813. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (see, e.g., Romanos et al., supra). Exemplary terminators for insect cells and mammalian cells include, but are not limited to, those from cytomegalovirus (CMV), Simian virus 40 (SV40), from Homo sapiens growth hormone hGH, from bovine growth hormone BGH, and from human or rabbit beta globulin.

In some embodiments, the control sequence is a suitable leader sequence, a non-translated region of an mRNA that is important for translation by the host cell. In some embodiments, the leader sequence is operably linked to the 5′ terminus of the nucleic acid sequence encoding the RNA polymerase polypeptide. Any suitable leader sequence that is functional in the host cell of choice find use in the present disclosure. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence is a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for filamentous fungal host cells include, but are not limited to the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (see, e.g., Guo and Sherman, Mol. Cell. Biol., 1995, 15:5983-5990). Useful polyadenylation and 3′ UTR sequences for mammalian host cells include, but are not limited to, the 3′-UTRs of α- and β-globin mRNAs that harbor several sequence elements that increase the stability and translation of mRNA.

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (see, e.g., Simonen and Palva, Microbiol. Rev., 1993, 57:109-137). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for mammalian host cells include but are not limited to those from the genes for immunoglobulin gamma (IgG) or other secreted human proteins, e.g., human beta-galactosidase polypeptide.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen.” A propolypeptide can be converted to a mature active polypeptide by catalytic or autocatalytic cleavage of the propeptide from the propolypeptide. The propeptide coding region may be obtained from any suitable source, including, but not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (see, e.g., WO95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, the control sequence comprises one or more regulatory sequences that facilitate regulation of the expression of the polynucleotide and/or corresponding encoded polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter. Exemplary inducible promoters regulated by exogenous agents include the zinc-inducible sheep metallothionine (MT) promoter, dexamethasone (Dex)-inducible promoter, mouse mammary tumor virus (MMTV) promoter; ecdysone insect promoter, tetracycline-inducible promoter system, RU486-inducible promoter system, and the rapamycin-inducible promoter system.

The recombinant expression vector may be any suitable vector (e.g., a plasmid or virus), that can be conveniently subjected to recombinant DNA procedures and bring about the expression of the engineered RNA polymerase. The choice of the vector typically is selected based on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication, e.g., origin of replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiment, the recombinant polynucleotides may be provided on a non-replicating expression vector or plasmid. In some embodiments, the non-replicating expression vector or plasmid can be based on viral vectors defective in replication (see, e.g., Travieso et al., npj Vaccines, 2022, Vol. 7, Article 75).

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol, or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof. Selectable marker for mammalian cells include, but are not limited to, chloramphenicol acetyl transferase (CAT), nourseothricin N-acetyl transferase, blasticidin-S deaminase, blastcidin S acetyltransferase, Sh ble (Zeocin® resistance), aminoglycoside 3′-phosphotransferase (neomycin resistance), hph (hygromycin resistance), thymidine kinase, and puromycin N-acetyl-transferase.

In another aspect, the present disclosure provides a host cell comprising a polynucleotide encoding at least one engineered RNA polymerase polypeptide of the present disclosure, the polynucleotide(s) being operably linked to one or more control sequences for expression of the engineered RNA polymerase polypeptide(s) in the host cell. In some embodiments, the host cell comprises an expression vector comprising a polynucleotide encoding an engineered RNA polymerase polypeptide described herein, where the polynucleotide is operably linked to one or more control sequences. Host cells suitable for use in expressing the polypeptides encoded by the expression vectors of the present disclosure are known in the art and include but are not limited to, bacterial cells, such as E. coli, B. subtilis, Vibrio fluvialis, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Exemplary host cells also include various Escherichia coli strains (e.g., W3110 (ΔfhuA) and BL21).

In another aspect, the present disclosure provides a method of producing the engineered RNA polymerase polypeptides, where the method comprises culturing a host cell capable of expressing a polynucleotide encoding the engineered RNA polymerase polypeptide under conditions suitable for expression of the polypeptide such that the engineered RNA polymerase is produced. In some embodiments, the method further comprises the step of isolating and/or purifying the RNA polymerase polypeptides, as described herein.

Appropriate culture media and growth conditions for host cells are known in the art. It is contemplated that any suitable method for introducing polynucleotides for expression of the RNA polymerase polypeptides into cells will find use in the present invention. Suitable techniques include, but are not limited to electroporation, biolistic particle bombardment, liposome mediated transfection, calcium chloride transfection, and protoplast fusion.

In some embodiments, the recombinant polypeptides (e.g., engineered RNA polymerase polypeptides) can be produced using any suitable methods known the art. For example, a wide variety of different mutagenesis techniques are available to the person of skill in the art. In addition, mutagenesis kits are also available from many commercial molecular biology suppliers. Methods are available to make specific substitutions at defined amino acids (site-directed), specific or random mutations in a localized region of the gene (region-specific), or random mutagenesis over the entire gene (e.g., saturation mutagenesis). Numerous methods known to those in the art to generate polypeptide variants, include, by way of example and not limitation, site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased activity, or low or reduced activity, increased thermal activity, increased stability, increased substrate range, increased fidelity, increased salt tolerance, and/or pH stability, etc.).

In some embodiments, the engineered RNA polymerase polypeptides with the properties disclosed herein can be obtained by subjecting the polynucleotide encoding the naturally occurring or engineered RNA polymerase polypeptide to a suitable mutagenesis and/or directed evolution methods known in the art, for example, as described herein. An exemplary directed evolution technique is mutagenesis and/or DNA shuffling (see, e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (see, e.g., Zhao et al., Nat. Biotechnol., 1998, 16:258-261), mutagenic PCR (see, e.g., Caldwell et al., PCR Methods Appl., 1994, 3:S136-S140), and cassette mutagenesis (see, e.g., Black et al., Proc. Natl. Acad. Sci. USA, 1996, 93:3525-3529).

Mutagenesis and directed evolution methods can be applied to RNA polymerase-encoding polynucleotides to generate variant libraries that can be expressed, screened, and assayed. Any suitable mutagenesis and directed evolution methods find use in the present disclosure and are known in the art (see, e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, 9,665,694, 9,684,771, and all related PCT and non-US counterparts; Ling et al., Anal. Biochem., 1997, 254(2):157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., Cell, 1984, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. U.S.A., 1997, 94:4504-4509; Crameri et al., Nat. Biotechnol., 1996, 14:315-319; Stemmer, Nature, 1994, 366:389-391; Stemmer, Proc. Nat. Acad. Sci. USA, 1994, 91:10747-10751; EP 3 049 973; WO 95/22625; WO 97/0078; WO 97/35966; WO 98/27230; WO 00/42651; WO 01/75767; WO 2009/152336; and WO 2015/048573, all of which are incorporated herein by reference).

In some embodiments, the clones obtained following mutagenesis treatment are screened by subjecting the polypeptide preparations to a defined treatment conditions or assay conditions (e.g., buffer, temperature, pH condition, DNA template, etc.) and measuring polypeptide activity after the treatments or other suitable assay conditions. Clones containing a polynucleotide encoding the polypeptide of interest are then isolated, the polynucleotide sequenced to identify the nucleotide sequence changes (if any), and used to express the polypeptide in a host cell. Measuring polypeptide activity from the expression libraries can be performed using any suitable method known in the art and as described in the Examples.

For engineered polypeptides of known sequence, the polynucleotides encoding the polypeptide can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence (see, e.g., Hughes et al., Cold Spring Harb Perspect Biol. 2017 January; 9(1):a023812). For example, polynucleotides and oligonucleotides disclosed herein can be prepared by chemical synthesis using the classical phosphoramidite method (see, e.g., Beaucage et al., Tet. Lett., 1981, 22:1859-69; and Matthes et al., EMBO J., 1984, 3:801-05), as it is typically practiced in automated synthetic methods. According to the phosphoramidite method, oligonucleotides are synthesized (e.g., in an automatic DNA synthesizer, purified, annealed, ligated, and cloned in appropriate vectors).

In some embodiments, a method for preparing the engineered RNA polymerase polypeptide can comprise: (a) synthesizing a polynucleotide encoding a polypeptide comprising an amino acid sequence selected from the amino acid sequence of any variant as described herein, and (b) expressing the RNA polymerase polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1- 45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions are conservative or non-conservative substitutions.

In some embodiments, any of the engineered RNA polymerase polypeptides expressed in a host cell are recovered and/or purified from the cells and/or the culture medium using any one or more of the known techniques for protein purification, including, among others, lysozyme treatment, sonication, filtration, salting-out, selective precipitation, ultra-centrifugation, and chromatography.

Chromatographic techniques for isolation and purification of the RNA polymerase polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying a particular polypeptide may depend, in part, on factors such as net charge, hydrophobicity, hydrophilicity, molecular weight, molecular shape, etc., and will be apparent to those having skill in the art. In some embodiments, affinity techniques may be used to isolate the improved RNA polymerase polypeptides. For affinity chromatography purification, any antibody that specifically binds an RNA polymerase polypeptide of interest can be used. For the production of antibodies, various host animals, including but not limited to rabbits, mice, rats, etc., are immunized by injection with an RNA polymerase polypeptide, or a fragment thereof. In some embodiments, the RNA polymerase polypeptide or fragment is attached to a suitable carrier, such as BSA, by means of a side chain functional group or linkers attached to a side chain functional group. Where the engineered RNA polymerase includes a fusion polypeptide that allows for affinity purification, such as a His-tag, standard affinity methods for the particular fusion protein can be used.

### Compositions

In a further aspect, the present disclosure provides compositions of the engineered RNA polymerases disclosed herein. In some embodiments, the engineered RNA polymerase polypeptide in the composition is isolated or purified. In some embodiments, the RNA polymerase is combined with other components and compounds to provide compositions and formulations comprising the engineered RNA polymerase polypeptide as appropriate for different applications and uses.

In some embodiments, the composition comprises at least one engineered RNA polymerase described herein. For example, a composition comprises at least one engineered RNA polymerase exemplified in Tables 5.1, 6.1, 7.1, 8.1, 9.1, 10.1, 11.1, 12.1, 13.1, 14.1, 15.1, 16.1, 17.1, 18.1, 19.1, 23.1 and 24.1, and the Sequence Listing. In some embodiments, the composition comprising an engineered RNA polymerase is an aqueous solution. In some embodiments, the composition comprising an engineered RNA polymerase is a lyophilizate.

In some embodiments, the composition further comprises one or more of (i) buffer, ii) one or more rNTPs, (iii) Mg⁺², and (iv) a target DNA substrate/template. In some embodiments, the target DNA substrate is a double stranded DNA template and includes a promoter recognized by the engineered RNA polymerase. As further described herein, in some embodiments, the promoter sequence is a naturally occurring T7 RNA polymerase promoter, such as, for example, a class II or class III promoter. In some embodiments, the RNA polymerase promoter is a synthetic or hybrid T7 RNA polymerase promoter.

In some embodiments, the composition further comprises a reducing agent, such as dithiothreitol or mercaptoethanol. In some embodiments, the composition further comprises an additive, such as a molecular crowding agent or a promoter of RNA polymerase activity. In some embodiments, the molecular crowding agent, includes, among others, bovine serum albumin (BSA), polyethylene glycol, dextran, and Ficoll. In some embodiments, the additive for promoting RNA polymerase activity is a surfactant, including, among others, non-ionic detergents, e.g., Triton X-100, Tween 20, or NP-40, and a poly-amine, e.g., spermidine or spermine.

In some embodiments, the composition further comprises one or more oligonucleotide primers, which can be sequence specific primers and/or “random” or “universal” primers that can act as primers for extension by the engineered RNA polymerase. In some embodiments, the primer is an oligoribonucleotide primer (i.e., RNA primer).

In some embodiments, the composition further comprises a “cap” analog, for example, for co-transcriptional production of capped RNA transcripts. In some embodiments, the cap analog is a dinucleotide or trinucleotide cap analog. In some embodiments, the cap analog is a dinucleotide cap analog, including, among others, alpha, gamma-bis(N7-methylguanosine) triphosphate (m7G(5′)ppp(5′)m7G) or an anti-reverse cap analog 3′-O-Me-m7G(5′)ppp(5′)G. In some embodiments, the dinucleotide cap analog is alpha, gamma-bis(N7-methylguanosine) triphosphate. In some embodiments, the cap analog is a trinucleotide cap analog, including, among others, m7G(5′)ppp(5′)AmpG (GAG) and m7,3′-O-propargylG(5′)ppp(5′)AmpG, m7GpppApA, m7GpppApC, m7GpppApG, m7GpppApU. m7GpppCpA, m7GpppCpC, m7GpppCpG, m7GpppCpU, m7GpppGpA, m7GpppGpC, m7GpppGpG, m7GpppGpU, m7CpppUpA, m7GpppUpC, m7GpppUpG, and m7GpppUpU. In some embodiments, the cap analog is a tetranucleotide cap, including, among others, (m7GpppNmpGmpG) and cap2-1 (m7GpppNpGmpG). Dinucleotide and trinucleotide cap analogs are described in, among others, Shanmugasundaram et al., Chem Rec., 2022, 22(8): e202200005, and PCT patent publication WO20172239, incorporated by reference herein. Tetranucleotide caps are described in, among others, Drazkowska et al., Nucl Acids Res., 2022, 50(16):9051-9071. In some embodiments, the cap analogs are commercially available cap analogs, sold s Trilink® or CleanCap®.

In some embodiments, the trinucleotide cap is m7G3′OMepppApA, m7G3′OMepppApC, m7G3′OMepppApG, m7G3′OMepppApU, m7G3′OmepppCpA, m7G3′OMepppCPC, m7G3′OMepppCpG, m7G3′OMepppCpU, m7G3′OMepppGpA, m7G3′OMepppGpC, m7G3′OMepppGpG, m7C3′OMepppGpU, m7G3′OMepppUpA, m7C3′OMepppUpC, m7G3′OMepppUpG, or m7G3′OMepppUpU. In some embodiments, the trinucleotide cap is m7G3′OMepppA2′OMepA, m7G3′OMepppA2′OMepC, m7G3′OMepppA2′OMepG, m7C3′OMepppA 2′OMepU, m7G3′OMepppC2′OMepA, m7C3′OMeppppC2′OMepC, m7C3′OMepppC2′OMepCm7G3′OMepppC2′OMepU, m7G3′OMepppG2′OMepA. m7G3′OMeppppG2′OMepC, m7G3′OMepppG2′OMepG, m7G 3′OMepppC2′OMepU, m7G3′OMepppU2′OMepA, m7G3′OMepppU2′OMepC, m7G3′OMepppU2′OMepG, or m7G3′OMepppU2′OMepU. In some embodiments, the trinucleotide cap is m7GpppA2′OMepA, m7GpppA2′OMepC, m7GpppA2′OMepG, m7GpppA2′OMepU, m7GpppC2′OMepA, m7GpppC2′OMepC, m7GpppC2′OMepG, m7CpppC2′OMepU, m7GpppG2′OMepA. m7GpppG2′OMepC, m7CpppG2′OMepG, m7GpppG2′OMepU, m7GpppU2′OMepA, m7GpppU2′OMepC, m7GpppU2′OMepG, or m7GpppU2′OMepU.

In some embodiments, the composition further comprises a pyrophosphatase and/or an RNase inhibitor. In some embodiments, the pyrophosphatase is an inorganic pyrophosphatase. In some embodiments, the RNase inhibitor is a mammalian RNase inhibitor (e.g., placental RNase inhibitor or porcine RNase inhibitor) or a synthetic RNase inhibitor, e.g., RiboGrip®.

In some embodiments, an engineered RNA polymerase described herein is provided immobilized on a substrate or support medium, such as a solid substrate, a porous substrate, a membrane, or particles. The polypeptide can be entrapped in matrixes or membranes. In some embodiments, matrices include polymeric materials such as calcium-alginate, agar, k-carrageenin, polyacrylamide, and collagen. In some embodiments, the solid matrices, includes, among others, activated carbon, porous ceramic, and diatomaceous earth. In some embodiments, the matrix is a particle, a membrane, or a fiber. Types of membranes include, among others, nylon, cellulose, polysulfone, or polyacrylate.

In some embodiments, the RNA polymerase is immobilized on the surface of a support material. In some embodiments, the polypeptide is adsorbed on the support material. In some embodiments, the polypeptide is immobilized on the support material by covalent attachment. Support materials include, among others, inorganic materials, such as alumina, silica, porous glass, ceramics, diatomaceous earth, clay, and bentonite, or organic materials, such as cellulose (CMC, DEAE-cellulose), starch, activated carbon, polyacrylate, agarose or derivatives thereof (e.g., cross-linked agarose), polyacrylamide, polystyrene, and ion-exchange resins, such as Amberlite, Sephadex, and Dowex.

### Uses and Methods

In another aspect, the present disclosure provides uses of the engineered RNA polymerases for preparing an RNA of interest. In some embodiments, the engineered RNA polymerase is used for in vitro transcription reactions. In some embodiments, the engineered RNA polymerase is used to produce, by way of example and not limitation, mRNA, self-replicating RNA, circular RNA, shRNA, miRNA, and CRISPR guide RNA. In embodiments, the RNA produced is mRNA that encodes a polypeptide of interest, such as a polypeptide vaccine or a therapeutic polypeptide.

In some embodiments, a method of producing RNA comprises contacting a target DNA template with an engineered RNA polymerase described herein in the presence of one or more nucleotide triphosphates (NTPs) under suitable reaction conditions such that an RNA transcript of all or part of the target DNA template is produced. In some embodiments, the method further comprises providing a cap analog, such as a dinucleotide, trinucleotide, or tetranucleotide cap, as described herein.

In some embodiments, the NTPs are rNTPs. In some embodiments, the NTP substrates are modified nucleotides. In some embodiments, the modified nucleotides are naturally modified nucleotides, for example the modified nucleotides of choice are the naturally occurring 5′-methylcytidine and/or pseudouridine. In some embodiments, the modified nucleotides are 2′-ribose modified nucleotides, for example 2-O-methyl, 2′-O-ethyl, and 2′-halo (e.g., 2′-bromo, 2′-fluoro). In some embodiments, the modified nucleotides are base-modified nucleotides (see, e.g., Milisavljevi6 et al., Org. Biomol. Chem., 2018, 16, 5800-5807).

In some embodiments, the concentration of one or more of the NTP, such as rNTP is 0.5-15 mM, 1-12 mM, 2-10 mM, or 4-8 mM. In some embodiments the concentration of NTPs is about 0.1 mM, 0.5 mM, 1 mM, 2 mM, 3 mM, 4 mM, 5 mM, 6 mM, 7 mM, 8 mM, 9 mM, 10 mM, 12 mM, 15 mM, or 20 mM.

In some embodiments, the suitable conditions comprise a temperature of about 20° C. to about 45° C. In some embodiments, the suitable conditions comprises a temperature of about 25° C. to about 40° C. In some embodiments, the suitable conditions comprises a temperature of about 25° C. to about 37° C. In some embodiments, the suitable conditions comprises a temperature of about 30° C. to about 35° C. In some embodiments, the suitable conditions comprises a temperature of about 20° C., 25° C., 30° C., 35° C., 37° C., 40° C., or 45° C.

In some embodiments, the suitable conditions include a buffer and/or Mg+2. In some embodiments, the suitable conditions include a reducing agent, for example, dithiothreitol or mercaptoethanol. In some embodiments, the suitable reaction conditions further include an additive, for example, bovine serum albumin (BSA), glycerol, polyethylene glycol, dextran, Ficoll, spermidine, or spermine, as described above.

In some embodiments, the target DNA template is a double stranded DNA template that includes a promoter sequence recognized by the engineered RNA polymerase described herein. In some embodiments, the promoter sequence is a naturally occurring T7 RNA polymerase promoter, such as, for example, a class II or class III promoter (see, e.g., Ikeda et al., J Biol. Chem., 1992, 267(4):2640-2649). In some embodiments, the promoter is a hybrid or synthetic T7 promoter recognized by the engineered RNA polymerase of the present disclosure (see, e.g., Lieber et al., Eur. J. Biochem. 1993, 217:387-394).

In some embodiments, where a cap has not been provided in the transcription reaction for co-transcriptional capping, the method further comprises the step of capping the RNA transcript. In some embodiments, capping of the RNA is carried out by a capping enzyme, for example, Faustovirus or vaccinia virus capping enzyme.

In some embodiments, the method further comprises the step of poly-A tailing of the RNA transcript. In some embodiments, the step of poly-A tailing is added when the polynucleotide template does not have a sequence for generating a poly-A tail in the RNA transcription. In some embodiments, the step of poly-A tailing uses a poly(A) polymerase, which catalyzes the incorporation of adenine residues into the 3′ termini of an RNA. In some embodiments, the poly(A) polymerase is a bacterial poly(A) polymerase (e.g., E. coli.), a yeast poly(A) polymerase (e.g., Saccharomyces cerevisiae), or a mammalian poly(A) polymerase.

In some embodiments, the method further comprises presence of a pyrophosphatase in conjunction with the engineered RNA polymerase. In general, the pyrophosphatase is an inorganic pyrophosphatase.

In some embodiments, the method further comprises presence of an RNase inhibitor for inhibiting RNases that may be present in the reaction mixture.

In some embodiments, the method is used in the production of an RNA transcript that encodes a polypeptide of interest. In some embodiments, the polypeptide of interest is an enzyme, vaccine antigen, cytokine, growth factor, monoclonal antibody, structural polypeptide or protein, or a ligand or receptor polypeptide or protein.

In some embodiments, the DNA template and the corresponding RNA transcript produced encode a vaccine antigen or therapeutic polypeptide. In some embodiments, the DNA template and the RNA transcript produced encodes a vaccine antigen for stimulating an immune response to the antigen, for example to treat a disease condition or stimulate immunity against an infectious agent.

In some embodiments the DNA template and the corresponding RNA transcript encode a vaccine antigen of a microbial or viral polypeptide. Exemplary microbial polypeptides include, among others, Streptococcus (e.g., Group A or Group B antigens) and plague bacterium proteins (e.g., F1 protein). Exemplary viral peptides include peptides of, among others, human immunodeficiency virus (HIV), hepatitis virus (e.g., HAV, HBV, HCV, etc.), herpes simplex virus (e.g., HSV-1, HSV-2, etc.), herpes zoster virus, human papilloma virus, respiratory syncytial virus (RSV), coronavirus (e.g., SARS-CoV-2), measles virus, pox virus (e.g., small pox, monkey pox, etc.), rhabdovirus (e.g., rabies), influenza (e.g., H1N1, H10N8, H7N9, etc.), human metapneumovirus (HMPV) and parainfluenza virus Type 3 (PIV3), human cytomegalovirus, Zika virus, Epstein-Barr Virus (EBV), and the like.

In some embodiments, the DNA template and the corresponding RNA transcript encode a cancer or tumor antigen. Exemplary cancer antigens include, among others, mRNA vaccines encoding tumor-associated antigens (TAAs), for example overexpressed antigens EGFR, HER2, cancer testis antigens (CTAs); differentiation antigens, for example. PSA and gp100; oncofoetal antigens, for example 5T4 and CEA; and onco-viral antigens, for example HPV E6 and E7 oncogenic proteins. In some embodiments, the cancer or tumor antigen is a tumor specific antigen (TSA) or tumor associated antigens (TAA) or neoantigens which are derived from somatic mutations. Exemplary TAA, TSA, and neoantigens include, among others, NY-ESO-1, MAGE-C3, tyrosinase, TPTE, RBL038, RBL039, RBL-040, RBL-041, RBL-045, PSA, PSCA, PSMA, STEAP1, PAP, and MUC1.

In some embodiments, the DNA template and the corresponding RNA transcript encode a therapeutic polypeptide. Exemplary therapeutic polypeptide includes, among others, an enzyme (e.g., associated with a disease condition), a cytokine, a structural polypeptide or protein, and a ligand or receptor polypeptide or protein.

Exemplary cytokines include, among others, IL-1β, IL-6, TNFα, IL-12, IL-15, GM-CSF, IFNα, and GM-CSF. Exemplary ligand proteins or polypeptides include, among others, OX40 ligand (OX40L), 4-1BB ligand (4-1BBL), glucocorticoid-induced tumor necrosis factor receptor (GITR) ligand (GITRL), CD40 ligand (CD40L), inducible T cell co-stimulatory ligand (ICOSL), CD70 and caTLR4.

Exemplary therapeutic polypeptides include, among others, glucose-6-phosphatase (glycogen storage deficiency type IA); phosphoenolpyruvatecarboxykinase (Pepck deficiency); galactose-1 phosphate uridyl transferase (galactosemia); phenylalanine hydroxylase (phenylketonuria); branched chain alpha-ketoacid dehydrogenase (Maple syrup urine disease); fumarylacetoacetate hydrolase (tyrosinemia type 1); methylmalonyl-CoA mutase (methylmalonic academia); medium chain acyl CoA dehydrogenase, (medium chain acetyl CoA deficiency); ornithine transcarbamylase (ornithine transcarbamylase deficiency); argininosuccinic acid synthetase (citrullinemia); low density lipoprotein receptor protein (familial hypercholesterolemia); UDP-glucouronosyltransferase (Crigler-Najjar disease); adenosine deaminase (severe combined immunodeficiency disease); hypoxanthine guanine phosphoribosyl transferase (Gout and Lesch-Nyan syndrome); biotinidase (biotinidase deficiency); beta-glucocerebrosidase (Gaucher disease); beta-glucuronidase (Sly syndrome); peroxisome membrane protein 70 kDa (Zellweger syndrome); porphobilinogen deaminase (acute intermittent porphyria); alpha-1 antitrypsin (alpha-1 antitrypsin deficiency; emphysema); Factor VIII (hemophilia A); and erythropoietin (thalassemia).

In some embodiments, the method is used to produce a non-coding RNA (ncRNA), for example, shRNA, siRNA, miRNA, iRNA, etc. In some embodiments, by way of example and not limitation, the non-coding RNA molecule is a miRNA which regulates gene expression by targeting RNA transcript cleavage/degradation or 5 translational repression of the target messenger RNA (mRNA). In some embodiments, the non-coding RNA is an shRNA or siRNA. The shRNA or siRNA can be directed to any target RNA (see, e.g., Zhang et al., Biochemical Pharmacology, 2021, 189:114432; and Schaefer et al., 2018, PLOS ONE 13(1): e0191570).

In some embodiments, the DNA template and expressed RNA is one or more components of a gene editing system or recombination system. In some embodiments, the DNA template and corresponding RNA encodes a CRISPR associated protein 9 (Cas9) or expresses a guide RNA (gRNA). In some embodiments, the DNA template and the corresponding RNA encode a zinc-finger nuclease (ZFN) or transcription factor-like effector nucleases (TALEN). In some embodiments, the transgene comprises a polynucleotide encoding a recombinase, such as Cre recombinase or flp recombinase.

In some embodiments, the engineered RNA polymerase is used to produce RNA for use as probes. In some embodiments, labeled nucleotide triphosphate substrates or nucleotide analogs can be used in the RNA polymerase reaction to incorporate the labeled nucleotide or nucleotide analog into the synthesized RNA.

In a further aspect, the present disclosure provides a kit comprising an engineered RNA polymerase or a composition thereof described herein. In some embodiments, the kit further comprises at least a buffer. In some embodiments, the buffer includes a reducing agent, e.g., dithiothreitol or mercaptoethanol.

In some embodiments, the kit further comprises an additive, such as glycerol, polyethylene glycol (e.g., PEG 6000 and PEG 8000), Triton X-100, dextran, bovine serum albumin (BSA), Ficoll, spermidine, spermine or dithiothreitol (DTT). In some embodiments, the kit further comprises on or more NTPs, particularly rNTPs. In some embodiments, the engineered RNA polymerase in the kit is provided as a lyophilizate or in solution.

### EXAMPLES

The following Examples, including experiments and results achieved, are provided for illustrative purposes only and are not to be construed as limiting the present invention.

### Example 1

### E. coli Expression Hosts Containing Recombinant RNA Polymerases (RNAP) Genes

The initial RNA polymerase enzyme used to produce the variants of the present invention was SEQ ID NO: 2 cloned into the expression vector pCK110900 (See, FIG. 3 of US Pat. Appln. Publn. No. 2006/0195947) operatively linked to the lac promoter under control of the lac1 repressor. The expression vector also contains the P15a origin of replication and the chloramphenicol resistance gene. The resulting plasmids were transformed into E. coli W3110, using standard methods known in the art. The transformants were isolated by subjecting the cells to chloramphenicol selection, as known in the art (See e.g., U.S. Pat. No. 8,383,346 and WO2010/144103).

### Example 2

### Preparation of HTP RNA Polymerases (RNAP)-Containing Wet Cell Pellets

E. coli cells containing recombinant RNAP-encoding genes from monoclonal colonies were inoculated into 180 μL LB containing 1% glucose and 30 μg/mL chloramphenicol (CAM) in the wells of 96-well, shallow-well microtiter plates. The plates were sealed with O₂-permeable seals, and cultures were grown overnight at 30° C., 200 rpm, and 85% humidity. Then, 10 μL of each of the cell cultures were transferred into the wells of 96-well, deep-well plates containing 390 mL Terrific Broth (TB) and 30 μg/mL chloramphenicol (CAM). The deep-well plates were sealed with O₂-permeable seals and incubated at 30° C., 250 rpm, and 85% humidity until OD₆₀₀ 0.6-0.8 was reached. The cell cultures were then induced by isopropylthiogalactoside (IPTG) to a final concentration of 1 mM and incubated overnight under the same conditions as originally used. The cells were then pelleted using centrifugation at 4,000 rpm for 10 min. The supernatants were discarded, and the pellets were frozen at −80° C. prior to lysis.

### Example 3

### Preparation of HTP Purified RNA Polymerases

First, 400 μL lysis buffer containing 50 mM sodium phosphate pH 7.5, 2 mM magnesium sulfate, 0.1% (v/v) Tween-20, 0.2 g/L lysozyme, 0.5 g/L polymyxin B sulfate (PMBS), and 0.5 mM dithiothreitol (DTT) was added to the cell paste in each well, produced as described in Example 2. The cells were shaken on a bench top shaker at room temperature for 2 h to resuspend and lyse. Then, 40 μL of 5 M NaCl and 5 μL of 0.8 M imidazole were added to the lysed cells, which were centrifuged for 10 min at 4,000 rpm at 4° C. The supernatants were loaded onto the HisPur™ Ni-NTA Spin Plates, washed twice with 600 μL of washing buffer containing 50 mM sodium phosphate pH 7.5, 300 mM sodium chloride, 0.1% (v/v) Tween-20, 10 mM imidazole, and 0.5 mM DTT, and eluted twice with 90 μL of elution buffer containing 50 mM sodium phosphate pH 7.5, 300 mM sodium chloride, 0.1% (v/v) Tween-20, 250 mM imidazole, and 0.5 mM DTT. Then, the HisPur™ plate eluent was buffer-exchanged into 2× storage buffer containing 100 Tris-HCl pH 8.0, 200 mM sodium chloride, 0.2% (v/v) Triton X-100, 2 mM EDTA pH 8.0, and 10 mM DTT via Zeba™ Spin Desalting Plate (40 K MWCO) following the manufacture protocol. 70 μL of the Zeba™ plate eluent was mixed with equal volume of pure glycerol and stored at −20° C. prior to activity assays.

### Example 4

### Shake Flask Expression and Purification of RNA Polymerases

### Shake Flask Expression

Selected variant cultures grown as described above were plated onto LB agar plates with 1% glucose and 30 μg/mL chloramphenicol and grown overnight at 37° C. A single colony from each culture was transferred to 5 mL Luria Broth (LB) broth with 1% glucose and 30 μg/ml chloramphenicol. The cultures were grown for 20 h at 30° C. and 250 rpm and subcultured at a dilution of approximately 1:50 into 250 ml Terrific Broth with 30 μg/ml of chloramphenicol, to a final OD₆₀₀ of about 0.05. The cultures were incubated for approximately 195 min at 30° C. and 250 rpm, to an OD₆₀₀ of about 0.6, and then induced with the addition of IPTG at a final concentration of 1 mM. The induced cultures were incubated for 20 h at 30° C. and 250 rpm. The cultures were the centrifuged at 4000 rpm for 10 min. The culture supernatant was discarded and the pellets were resuspended in lysis buffer comprised of 50 mM sodium phosphate at pH 7.5, 10 mM imidazole, 500 mM sodium chloride, and 0.1% (v/v) Tween-20. This cell suspension was chilled in an ice bath and lysed using a Microfluidizer cell disruptor (Microfluidics M-110L). The crude lysate was pelleted by centrifugation (11,000 rpm for 60 min at 4° C.), and the supernatant was then filtered through a 0.2 μm PES membrane to further clarify the lysate.

Purification of RNAP from Shake Flask Lysates

Additional NaCl and imidazole were added to the clarified RNAP lysates to adjust the concentrations to 500 mM NaCl and 20 mM imidazole. Lysates were then purified using an AKTA Pure purification system and a 5 ml HisTrap FF column (GE Healthcare) using the AC Step HiF setting (the run parameters are provided below in Table 4.1). The wash buffer comprised 50 mM sodium phosphate pH 7.5, 500 mM sodium chloride, 25 mM imidazole, and 0.1% (v/v) Tween-20, and the elution buffer comprised 50 mM sodium phosphate pH 7.5, 500 mM sodium chloride, 300 mM imidazole, and 0.1% (v/v) Tween-20.

TABLE 4.1
Purification Parameters

| Parameter | Volume | |
|---|---|---|
| Column volume | 5 | mL |
| Flow rate | 5 | mL/min |
| Pressure limit | 0.3 | MPa |
| Sample volume | 45 | ml |
| Equilibration volume | 5 column volumes (CV) = 25 mL | |
| Wash Unbound volume | 15 CV = 75 mL | |
| Elution | Isocratic (step) | |
| Elution volume | 5 CV = 25 mL | |
| Fraction volume | 1.5 | mL |

| | |
|---|---|
| RE-equilibration volume | 5 CV = 25 mL |

The first 2 elution fractions with over 1 mg/mL protein were identified by absorption at 280 nm on a NanoDrop Microvolume Spectrophotometers and dialyzed overnight in dialysis buffer comprised of 25 mM HEPES pH 7.6, 150 mM sodium chloride, 0.1 mM EDTA, 0.09% Triton X-100, 10 mM dithiothreitol (DTT), and 50% glycerol in a 3.5 K Slide-A-Lyzer™ dialysis cassette (Thermo Fisher) for buffer exchange. Concentrations of purified and dialyzed RNA polymerase samples were measured by absorption at 280 nm.

### Example 5

### Improvements Over RNA Polymerase of SEQ ID NO: 2

SEQ ID NO: 2 was selected as the parent enzyme in an in vitro transcription (IVT) assay. Libraries of engineered genes were produced using known techniques (e.g., saturation mutagenesis, recombination of previously identified beneficial mutations). The polypeptides encoded by each gene were produced in HTP as described in Example 2 and purified as described in Example 3. Each variant was diluted 4-fold in RNase-free water and screened in a 1 μL reaction that comprised 0.05 μg/μL 9 kb-DNA template, 30 mM Tris-HCl pH 8.0, 26.9 mM magnesium chloride, 5 mM ATP, 5 mM GTP, 5 mM CTP, 5 mM UTP, 1 U/μL RNase inhibitor (New England Biolabs, Catalog #M0314L), and 0.002 U/μL IPPase (New England Biolabs, Catalog #M2403L). The reactions were initiated by adding 150 nL of the HTP purified variants, incubated in Bio-Rad Hard-Shell® 96-Well PCR plates at 37° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 2 (Activity Fold Improvement Over Parent, FIOP) was calculated as the RNA produced by variant over the RNA produced by SEQ ID NO: 2. The result is shown in Table 5.1.

TABLE 5.1
Activities of Variants Relative to SEQ ID NO: 2

| SEQ ID | | FIOP Product |
|---|---|---|
| NO: | Amino Acid Differences | Relative to |
| (nt/aa) | (Relative toSEQ ID NO: 2) | SEQ ID NO: 2 |
| 3/4 | V401R/Y404L | +++ |
| 5/6 | K336S | +++ |
| 7/8 | N138R | ++ |
| 9/10 | K336V | ++ |
| 11/12 | V401R/I403V/Y404S | ++ |
| 13/14 | K336M/A510V | + |
| 15/16 | K336L | + |
| 17/18 | A388K | + |
| 19/20 | K340E | ++ |
| 21/22 | V401R/R402T/Y404S | + |
| 23/24 | W351L/R357E | + |
| 25/26 | V401R/Y404I | + |

Levels of increased activity were determined relative to the reference polypeptide of SEQ ID NO: 2 and defined as follows: ″″+″″ 1.01 to 1.10 (first 50%), ″″++″″ >1.10 (next 30%), ″″+++″″ >1.18 (top 20%)

### Example 6

### Improvements Over RNA Polymerase of SEQ ID NO: 4

SEQ ID NO: 4 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 9 kb-DNA template, 40 mM Tris-HCl pH 8.0, 30 mM magnesium acetate, 5 mM ATP, 5 mM GTP, 5 mM CTP, 5 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 37° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 4 (Activity FIOP) was calculated as the RNA produced by variant over the RNA produced by SEQ ID NO: 4. The result is shown in Table 6.1.

TABLE 6.1
Activities of Variants Relative to SEQ ID NO: 4

| SEQ ID | | | FIOP Product |
|---|---|---|---|
| NO: | Amino Acid Differences | Amino Acid Differences | Relative to |
| (nt/aa) | (Relative to SEQ ID NO: 4) | (Relative to SEQ ID NO: 2) | SEQ ID NO: 4 |
| 27/28 | A311G/A388K/L404Y/N440K | A311G/A388K/V401R/N440K | +++ |
| 29/30 | N138R/K340W | N138R/K340W/V401R/Y404L | ++ |
| 31/32 | N138R/A311G/K336S/K340W/A388K | N138R/A311G/K336S/K340W/A388K/ | ++ |
| | | V401R/Y404L | |
| 33/34 | A311G/A388K/N440K | A311G/A388K/V401R/Y404L/N440K | + |
| 35/36 | N138R/A311G/K340E/A388K/L404Y/ | N138R/A311G/K340E/A388K/V401R/ | + |
| | N440K | N440K | |
| 37/38 | N138R/K336M/L342Q | N138R/K336M/L342Q/V401R/Y404L | + |
| 39/40 | W351L/A388K | W351L/A388K/V401R/Y404L | + |

Levels of increased activity were determined relative to the reference polypeptide of SEQ ID NO: 4 and defined as follows: ″″+″″ 1.01 to 1.10 (first 50%), ″″++″″ >1.10 (next 30%), ″″+++″″ >1.18 (top 20%)

### Example 7

### Improvements Over RNA Polymerase of SEQ ID NO: 32

SEQ ID NO: 32 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HTP as described in Example 2 and purified as described in Example 3. Each variant was diluted 4-fold in RNase-free water and screened in a 1 μL reaction that comprised 0.05 μg/μL 8 kb-DNA template, 40 mM Tris-HCl pH 8.0, 30 mM magnesium acetate, 7.5 mM ATP, 7.5 mM GTP, 7.5 mM CTP, 7.5 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HTP purified variants, incubated in Bio-Rad Hard-Shell® 96-Well PCR plates at 37° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 32 (Activity FIOP) was calculated as the RNA produced by variant over the RNA produced by SEQ ID NO: 32. The result is shown in Table 7.1.

TABLE 7.1
Activities of Variants Relative to SEQ ID NO: 32

| | | | FIOP Product |
|---|---|---|---|
| SEQ ID | | | Relative to |
| NO: | Amino Acid Differences | Amino Acid Differences | SEQ ID NO: |
| (nt/aa) | (Relative to SEQ ID NO: 32) | (Relative to SEQ ID NO: 2) | 32 |
| 41/42 | L514S | N138R/A311G/K336S/K340W/A388K/V401R/ | ++ |
| | | Y404L/L514S | |
| 43/44 | R38G/L514S | R38G/N138R/A311G/K336S/K340W/A388K/ | + |
| | | V401R/Y404L/L514S | |
| 45/46 | R38G/R394K/L514S | R38G/N138R/A311G/K336S/K340W/A388K/ | + |
| | | R394K/V401R/Y404L/L514S | |
| 47/48 | R38G/R394K/L514S/ | R38G/N138R/A311G/K336S/K340W/A388K/ | + |
| | W664P | R394K/V401R/Y404L/L514S/W664P | |
| 49/50 | R394K/L514S/G875C | N138R/A311G/K336S/K340W/A388K/R394K/ | + |
| | | V401R/Y404L/L514S/G875C | |
| 51/52 | R394K/L514S | N138R/A311G/K336S/K340W/A388K/R394K/ | + |
| | | V401R/Y404L/L514S | |
| 53/54 | R38G/I124F/R394K/ | R38G/I124F/N138R/A311G/K336S/K340W/ | + |
| | L514I | A388K/R394K/V401R/Y404L/L514I | |
| 55/56 | K171E/N878M | N138R/K171E/A311G/K336S/K340W/A388K/ | +++ |
| | | V401R/Y404L/N878M | |
| 57/58 | K170Q | N138R/K170Q/A311G/K336S/K340W/A388K/ | +++ |
| | | V401R/Y404L | |
| 59/60 | V173Y | N138R/V173Y/A311G/K336S/K340W/A388K/ | +++ |
| | | V401R/Y404L | |
| 61/62 | N172V | N138R/N172V/A311G/K336S/K340W/A388K/ | +++ |
| | | V401R/Y404L | |
| 63/64 | V173L | N138R/V173L/A311G/K336S/K340W/A388K/ | ++ |
| | | V401R/Y404L | |
| 65/66 | N172E | N138R/N172E/A311G/K336S/K340W/A388K/ | ++ |
| | | V401R/Y404L | |
| 67/68 | K170S/A629D | N138R/K170S/A311G/K336S/K340W/A388K/ | ++ |
| | | V401R/Y404L/A629D | |
| 69/70 | K171F | N138R/K171F/A311G/K336S/K340W/A388K/ | ++ |
| | | V401R/Y404L | |
| 71/72 | H168V | N138R/H168V/A311G/K336S/K340W/A388K/ | ++ |
| | | V401R/Y404L | |
| 73/74 | N172K | N138R/N172K/A311G/K336S/K340W/A388K/ | ++ |
| | | V401R/Y404L | |
| 75/76 | A166R | N138R/A166R/A311G/K336S/K340W/A388K/ | ++ |
| | | V401R/Y404L | |
| 77/78 | H168K | N138R/H168K/A311G/K336S/K340W/A388K/ | ++ |
| | | V401R/Y404L | |
| 79/80 | A166V/S272N/M274L/ | N138R/A166V/S272N/M274L/F275Y/A311G/ | ++ |
| | F275Y | K336S/K340W/A388K/V401R/Y404L | |
| 81/82 | K170R | N138R/K170R/A311G/K336S/K340W/A388K/ | ++ |
| | | V401R/Y404L | |
| 83/84 | F169L | N138R/F169L/A311G/K336S/K340W/A388K/ | ++ |
| | | V401R/Y404L | |
| 85/86 | A207S | N138R/A207S/A311G/K336S/K340W/A388K/ | ++ |
| | | V401R/Y404L | |
| 87/88 | F169G | N138R/F169G/A311G/K336S/K340W/A388K/ | + |
| | | V401R/Y404L | |
| 89/90 | H168L/I217P/H218Q | N138R/H168L/I217P/H218Q/A311G/K336S/ | + |
| | | K340W/A388K/V401R/Y404L | |
| 91/92 | H168W | N138R/H168W/A311G/K336S/K340W/A388K/ | + |
| | | V401R/Y404L | |
| 93/94 | N172R | N138R/N172R/A311G/K336S/K340W/A388K/ | + |
| | | V401R/Y404L | |
| 95/96 | A166D | N138R/A166D/A311G/K336S/K340W/A388K/ | + |
| | | V401R/Y404L | |
| 97/98 | K171L | N138R/K171L/A311G/K336S/K340W/A388K/ | + |
| | | V401R/Y404L | |
| 99/100 | K171Y/H530N | N138R/K171Y/A311G/K336S/K340W/A388K/ | + |
| | | V401R/Y404L/H530N | |
| 101/102 | N172A | N138R/N172A/A311G/K336S/K340W/A388K/ | + |
| | | V401R/Y404L | |
| 103/104 | V173H | N138R/V173H/A311G/K336S/K340W/A388K/ | + |
| | | V401R/Y404L | |
| 105/106 | T382R | N138R/A311G/K336S/K340W/T382R/A388K/ | + |
| | | V401R/Y404L | |
| 107/108 | D773A | N138R/A311G/K336S/K340W/A388K/V401R/ | + |
| | | Y404L/D773A | |
| 109/110 | H168R | N138R/H168R/A311G/K336S/K340W/A388K/ | + |
| | | V401R/Y404L | |
| 111/112 | K670R | N138R/A311G/K336S/K340W/A388K/V401R/ | + |
| | | Y404L/K670R | |
| 113/114 | H168T | N138R/H168T/A311G/K336S/K340W/A388K/ | + |
| | | V401R/Y404L | |
| 115/116 | N172G | N138R/N172G/A311G/K336S/K340W/A388K/ | + |
| | | V401R/Y404L | |
| 117/118 | E379A | N138R/A311G/K336S/K340W/E379A/A388K/ | + |
| | | V401R/Y404L | |
| 119/120 | Y392M | N138R/A311G/K336S/K340W/A388K/Y392M/ | + |
| | | V401R/Y404L | |
| 121/122 | K419R | N138R/A311G/K336S/K340W/A388K/V401R/ | + |
| | | Y404L/K419R | |
| 123/124 | Y392R | N138R/A311G/K336S/K340W/A388K/Y392R/ | + |
| | | V401R/Y404L | |
| 125/126 | Y392A | N138R/A311G/K336S/K340W/A388K/Y392A/ | + |
| | | V401R/Y404L | |
| 127/128 | R57G | R57G/N138R/A311G/K336S/K340W/A388K/ | + |
| | | V401R/Y404L | |
| 129/130 | H168S | N138R/H168S/A311G/K336S/K340W/A388K/ | + |
| | | V401R/Y404L | |
| 131/132 | L164W | N138R/L164W/A311G/K336S/K340W/A388K/ | + |
| | | V401R/Y404L | |
| 133/134 | K670Q | N138R/A311G/K336S/K340W/A388K/V401R/ | + |
| | | Y404L/K670Q | |
| 135/136 | G810K | N138R/A311G/K336S/K340W/A388K/V401R/ | + |
| | | Y404L/G810K | |
| 137/138 | T382G | N138R/A311G/K336S/K340W/T382G/A388K/ | + |
| | | V401R/Y404L | |
| 139/140 | E659R | N138R/A311G/K336S/K340W/A388K/V401R/ | + |
| | | Y404L/E659R | |
| 141/142 | G54H | G54H/N138R/A311G/K336S/K340W/A388K/ | + |
| | | V401R/Y404L | |
| 143/144 | K419S | N138R/A311G/K336S/K340W/A388K/V401R/ | + |
| | | Y404L/K419S | |
| 145/146 | N172E/V173Y/L514S/ | N138R/N172E/V173Y/A311G/K336S/K340W/ | +++ |
| | E659R/D660H | A388K/V401R/Y404L/L514S/E659R/D660H | |
| 147/148 | K170Q/K171E/N172E/ | N138R/K170Q/K171E/N172E/A311G/K336S/ | +++ |
| | L514S/E659R | K340W/A388K/V401R/Y404L/L514S/E659R | |
| 149/150 | V173Y/L514S/E659R/ | N138R/V173Y/A311G/K336S/K340W/A388K/ | +++ |
| | D660H | V401R/Y404L/L514S/E659R/D660H | |
| 151/152 | V173Y/L514S/E659R/ | N138R/V173Y/A311G/K336S/K340W/A388K/ | +++ |
| | G810K | V401R/Y404L/L514S/E659R/G810K | |
| 153/154 | K170Q/W340K/L514S/ | N138R/K170Q/A311G/K336S/A388K/V401R/ | +++ |
| | E659R/D660H/G810K | Y404L/L514S/E659R/D660H/G810K | |
| 155/156 | K170Q/K171E/V173Y/ | N138R/K170Q/K171E/V173Y/A311G/A388K/ | +++ |
| | S336K/W340K/L514S/ | V401R/Y404L/L514S/E659R/D660H | |
| | E659R/D660H | | |
| 157/158 | R57G/K171E/V173Y/ | R57G/N138R/K171E/V173Y/A311G/K336S/K340W/ | +++ |
| | L514S/E659R/D660H/ | A388K/V401R/Y404L/L514S/E659R/D660H/ | |
| | G810K | G810K | |
| 159/160 | K170Q/V173Y/L514S/ | N138R/K170Q/V173Y/A311G/K336S/K340W/A388K/ | +++ |
| | E659R/D660H/G810K | V401R/Y404L/L514S/E659R/D660H/G810K | |
| 161/162 | R57G/K171E/N172K/S336K/ | R57G/N138R/K171E/N172K/A311G/A388K/V401R/ | +++ |
| | W340K/L514S/E659R | Y404L/L514S/E659R | |
| 163/164 | V173Y/L514S | N138R/V173Y/A311G/K336S/K340W/A388K/V401R/ | +++ |
| | | Y404L/L514S | |
| 165/166 | K171E/N172E/V173Y/ | N138R/K171E/N172E/V173Y/A311G/K336S/K340W/ | +++ |
| | L514S/E659R/D660H | A388K/V401R/Y404L/L514S/E659R/D660H | |
| 167/168 | R57G/K170Q/K171E/ | R57G/N138R/K170Q/K171E/V173Y/A311G/K336S/ | ++ |
| | V173Y/L514S/E659R/ | K340W/A388K/V401R/Y404L/L514S/E659R/D660H/ | |
| | D660H/G810K | G810K | |
| 169/170 | R57G/V173Y/L514S | R57G/N138R/V173Y/A311G/K336S/K340W/A388K/ | ++ |
| | | V401R/Y404L/L514S | |
| 171/172 | N172K/V173D/L514S/ | N138R/N172K/V173D/A311G/K336S/K340W/A388K/ | ++ |
| | A857V | V401R/Y404L/L514S/A857V | |
| 173/174 | R57G/V173Y/W340K/ | R57G/N138R/V173Y/A311G/K336S/A388K/V401R/ | ++ |
| | L514S | Y404L/L514S | |
| 175/176 | K170Q/K171E/V173Y/ | N138R/K170Q/K171E/V173Y/A311G/K340W/A388K/ | ++ |
| | S336K/L514S | V401R/Y404L/L514S | |
| 177/178 | K171E/L514S | N138R/K171E/A311G/K336S/K340W/A388K/V401R/ | ++ |
| | | Y404L/L514S | |
| 179/180 | R57G/K170Q/V173Y/ | R57G/N138R/K170Q/V173Y/A311G/K340W/A388K/ | ++ |
| | S336K/L514S | V401R/Y404L/L514S | |
| 181/182 | K170Q/S336K | N138R/K170Q/A311G/K340W/A388K/V401R/Y404L | ++ |
| 183/184 | V173Y/L514S/E659R | N138R/V173Y/A311G/K336S/K340W/A388K/V401R/ | ++ |
| | | Y404L/L514S/E659R | |
| 185/186 | R57G/L514S | R57G/N138R/A311G/K336S/K340W/A388K/V401R/ | + |
| | | Y404L/L514S | |
| 187/188 | V173Y/G810K | N138R/V173Y/A311G/K336S/K340W/A388K/V401R/ | + |
| | | Y404L/G810K | |

Levels of increased activity were determined relative to the reference polypeptide of SEQ ID NO: 32 and defined as follows: ″+″ 1.04 to 2.30 (first 50%), ″++″ >2.30 (next 30%), ″+++″ >5.31 (top 20%)

### Example 8

### Improvements Over RNA Polymerase of SEQ ID NO: 44

SEQ ID NO: 44 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HTP as described in Example 2 and purified as described in Example 3. Each variant was diluted 4-fold in RNase-free water and screened in a 1 μL reaction that comprised 0.05 μg/μL 8 kb-DNA template, 40 mM Tris-HCl pH 8.0, 30 mM magnesium acetate, 7.5 mM ATP, 7.5 mM GTP, 7.5 mM CTP, 7.5 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HTP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 44 (Activity FIOP) was calculated as the RNA produced by variant over the RNA produced by SEQ ID NO: 44. The result is shown in Table 8.1.

TABLE 8.1
Activities of Variants Relative to SEQ ID NO: 44

| | | | FIOP Product |
|---|---|---|---|
| SEQ ID | | | Relative to |
| NO: | Amino Acid Differences | Amino Acid Differences | SEQ ID NO: |
| (nt/aa) | (Relative to SEQ ID NO: 44) | (Relative to SEQ ID NO: 2) | 44 |
| 189/190 | S336K | R38G/N138R/A311G/K340W/A388K/ | + |
| | | V401R/Y404L/L514S | |
| 191/192 | A143V/S336K/W340K/E782V | R38G/N138R/A143V/A311G/A388K/ | + |
| | | V401R/Y404L/L514S/E782V | |
| 193/194 | A143V/S336K/W340K/R357E/ | R38G/N138R/A143V/A311G/R357E/ | + |
| | S514L | A388K/V401R/Y404L | |

Levels of increased activity were determined relative to the reference polypeptide of SEQ ID NO: 44 and defined as follows: ″+″ from 1.60 to 2.08

### Example 9

### Improvements Over RNA Polymerase of SEQ ID NO: 156

SEQ ID NO: 156 was selected as the parent enzyme in the in vitro transcription (IV) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 8 kb-DNA template, 40 mM Tris-HCl pH 8.0, 42 mM magnesium acetate, 10 mM ATP, 10 mM GTP, 10 mM CTP, 10 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 156 (Activity FIOP) was calculated as the RNA produced by variant over the RNA produced by SEQ ID NO: 156. The result is shown in Table 9.1.

TABLE 9.1
Activities of Variants Relative to SEQ ID NO: 156

| | | | FIOP Product |
|---|---|---|---|
| SEQ ID | | | Relative to |
| NO: | Amino Acid Differences | Amino Acid Differences | SEQ ID NO: |
| (nt/aa) | (Relative to SEQ ID NO: 156) | (Relative to SEQ ID NO: 2) | 156 |
| 195/196 | A207S | N138R/V173Y/A207S/A311G/K336S/K340W/ | +++ |
| | | A388K/V401R/Y404L/L514S/E659R/D660H | |
| 197/198 | K419R | N138R/V173Y/A311G/K336S/K340W/A388K/ | +++ |
| | | V401R/Y404L/K419R/L514S/E659R/D660H | |
| 199/200 | A207S/S336K/W340K | N138R/V173Y/A207S/A311G/A388K/V401R/ | +++ |
| | | Y404L/L514S/E659R/D660H | |
| 201/202 | A166R/F169L | N138R/A166R/F169L/V173Y/A311G/K336S/ | ++ |
| | | K340W/A388K/V401R/Y404L/L514S/E659R/ | |
| | | D660H | |
| 203/204 | A207S/K670R | N138R/V173Y/A207S/A311G/K336S/K340W/ | ++ |
| | | A388K/V401R/Y404L/L514S/E659R/D660H/ | |
| | | K670R | |
| 205/206 | A166R/S336K/W340K | N138R/A166R/V173Y/A311G/A388K/V401R/ | ++ |
| | | Y404L/L514S/E659R/D660H | |
| 207/208 | A207S/K419R/K670R | N138R/V173Y/A207S/A311G/K336S/K340W/ | ++ |
| | | A388K/V401R/Y404L/K419R/L514S/E659R/ | |
| | | D660H/K670R | |
| 209/210 | A207S/S336K/W340K/ | N138R/V173Y/A207S/A311G/T382G/A388K/ | ++ |
| | T382G/K419R/K670R | V401R/Y404L/K419R/L514S/E659R/D660H/ | |
| | | K670R | |
| 211/212 | A207S/T382G | N138R/V173Y/A207S/A311G/K336S/K340W/ | ++ |
| | | T382G/A388K/V401R/Y404L/L514S/E659R/ | |
| | | D660H | |
| 213/214 | K670R | N138R/V173Y/A311G/K336S/K340W/A388K/ | + |
| | | V401R/Y404L/L514S/E659R/D660H/K670R | |
| 215/216 | S336K/K670R | N138R/V173Y/A311G/K340W/A388K/V401R/ | + |
| | | Y404L/L514S/E659R/D660H/K670R | |
| 217/218 | W340K/K670R | N138R/V173Y/A311G/K336S/A388K/V401R/ | + |
| | | Y404L/L514S/E659R/D660H/K670R | |
| 219/220 | T382G | N138R/V173Y/A311G/K336S/K340W/T382G/ | + |
| | | A388K/V401R/Y404L/L514S/E659R/D660H | |
| 221/222 | K127G/V141A/A207S/ | K127G/N138R/V141A/V173Y/A207S/A311G/ | + |
| | S336K/T382G/K670R | K340W/T382G/A388K/V401R/Y404L/L514S/ | |
| | | E659R/D660H/K670R | |
| 223/224 | A207S/S336K/T382G/ | N138R/V173Y/A207S/A311G/K340W/T382G/ | + |
| | K419R/K670R | A388K/V401R/Y404L/K419R/L514S/E659R/ | |
| | | D660H/K670R | |
| 225/226 | K419R/K670R | N138R/V173Y/A311G/K336S/K340W/A388K/ | + |
| | | V401R/Y404L/K419R/L514S/E659R/D660H/ | |
| | | K670R | |
| 227/228 | A166R/H168V/A207S/S336K/ | N138R/A166R/H168V/V173Y/A207S/A311G/ | + |
| | W340K/K419R/K670R | A388K/V401R/Y404L/K419R/L514S/E659R/ | |
| | | D660H/K670R | |
| 229/230 | A166V/W340K | N138R/A166V/V173Y/A311G/K336S/A388K/ | + |
| | | V401R/Y404L/L514S/E659R/D660H | |
| 231/232 | A207S/S336K/W340K/ | N138R/V173Y/A207S/A311G/A388K/V401R/ | + |
| | K670R | Y404L/L514S/E659R/D660H/K670R | |

Levels of increased activity were determined relative to the reference polypeptide of SEQ ID NO: 156 and defined as follows: ″+″ 1.04 to 1.26 (first 50%), ″++″ >1.26 (next 30%), ″+++″ >1.90 (top 20%)

### Example 10

### Improvements Over RNA Polymerase of SEQ ID NO: 156: Thermostability

SEQ ID NO: 156 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HTP as described in Example 2 and purified as described in Example 3. Each variant was diluted 4-fold in RNase-free water, preincubated at 33° C. for 30 min, and screened in a 1 μL reaction that comprised 0.05 μg/μL 8 kb-DNA template, 40 mM Tris-HCl pH 8.0, 30 mM magnesium acetate, 7.5 mM ATP, 7.5 mM GTP, 7.5 mM CTP, 7.5 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HTP purified variants, in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 156 (Activity FIOP) was calculated as the RNA produced by variant over the RNA produced by SEQ ID NO: 156, which was smaller than 1 and therefore rounded up to 1. The result is shown in Table 10.1.

TABLE 10.1
Activities of Variants Relative to SEQ ID NO: 156 after 33° C. Heat Challenge for 30 min

| | | | FIOP Product |
|---|---|---|---|
| SEQ ID | | | Relative to |
| NO: | Amino Acid Differences | Amino Acid Differences | SEQ ID NO: |
| (nt/aa) | (Relative to SEQ ID NO: 156) | (Relative to SEQ ID NO: 2) | 156 |
| 233/234 | R38G/S437P/H779R | R38G/N138R/V173Y/A311G/K336S/K340W/A388K/ | +++ |
| | | V401R/Y404L/S437P/L514S/E659R/D660H/ | |
| | | H779R | |
| 235/236 | R38G/S437P | R38G/N138R/V173Y/A311G/K336S/K340W/A388K/ | +++ |
| | | V401R/Y404L/S437P/L514S/E659R/D660H | |
| 237/238 | R38G | R38G/N138R/V173Y/A311G/K336S/K340W/A388K/ | +++ |
| | | V401R/Y404L/L514S/E659R/D660H | |
| 239/240 | R38G/A207S/S437P/H779R | R38G/N138R/V173Y/A207S/A311G/K336S/K340W/ | ++ |
| | | A388K/V401R/Y404L/S437P/L514S/E659R/D660H/ | |
| | | H779R | |
| 241/242 | C517R/H779R | N138R/V173Y/A311G/K336S/K340W/A388K/V401R/ | ++ |
| | | Y404L/L514S/C517R/E659R/D660H/H779R | |
| 243/244 | R38G/S437P/G527E/W664K | R38G/N138R/V173Y/A311G/K336S/K340W/A388K/ | ++ |
| | | V401R/Y404L/S437P/L514S/G527E/E659R/D660H/ | |
| | | W664K | |
| 245/246 | R38G/A207S/E379S/S437P | R38G/N138R/V173Y/A207S/A311G/K336S/K340W/ | ++ |
| | | E379S/A388K/V401R/Y404L/S437P/L514S/E659R/ | |
| | | D660H | |
| 247/248 | W664K/K670R/H779R | N138R/V173Y/A311G/K336S/K340W/A388K/V401R/ | + |
| | | Y404L/L514S/E659R/D660H/W664K/K670R/ | |
| | | H779R | |
| 249/250 | R38G/H779R | R38G/N138R/V173Y/A311G/K336S/K340W/A388K/ | + |
| | | V401R/Y404L/L514S/E659R/D660H/H779R | |
| 251/252 | R38G/S437P/C517R | R38G/N138R/V173Y/A311G/K336S/K340W/A38K/8 | + |
| | | V401R/Y404L/S437P/L514S/C517R/E659R/D660H | |
| 253/254 | S437P/H779R | N138R/V173Y/A311G/K336S/K340W/A388K/V401R/ | + |
| | | Y404L/S437P/L514S/E659R/D660H/H779R | |
| 255/256 | R38G/S437P/E607Y | R38G/N138R/V173Y/A311G/K336S/K340W/A388K/ | + |
| | | V401R/Y404L/S437P/L514S/E607Y/E659R/D660H | |
| 257/258 | R38G/E379S/H779R | R38G/N138R/V173Y/A311G/K336S/K340W/E379S/ | + |
| | | A388K/V401R/Y404L/L514S/E659R/D660H/H79R7 | |
| 259/260 | C517R/K670R/H779R | N138R/V173Y/A311G/K336S/K340W/A388K/V401R/ | + |
| | | Y404L/L514S/C517R/E659R/D660H/K670R/H779R | |

Levels of increased activity after preincubation at 33° C. for 30 min were determined relative to the reference polypeptide of SEQ ID NO: 156 and defined as follows: ″+″ 1.00 to 1.33 (first 50%), ″++″ >1.33 (next 30%), ″+++″ >1.71 (top 20%)

### Example 11

### Improvements Over RNA Polymerase of SEQ ID NO: 234

SEQ ID NO: 234 was selected as the parent enzyme in the in vitro transcription (IV) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 8 kb-DNA template, 40 mM Tris-HCl pH 8.0, 42 mM magnesium acetate, 10 mM ATP, 10 mM GTP, 10 mM CTP, 10 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 234 (Activity FIOP) was calculated as the RNA produced by variant over the RNA produced by SEQ ID NO: 234. The result is shown in Table 11.1.

TABLE 11.1
Activities of Variants Relative to SEQ ID NO: 234

| | Amino Acid | | FIOP Product |
|---|---|---|---|
| SEQ ID | Differences | | Relative to |
| NO: | (Relative to | Amino Acid Differences | SEQ ID NO: |
| (nt/aa) | SEQ ID NO: 234) | (Relative to SEQ ID NO: 2) | 234 |
| 261/262 | K720A | R38G/N138R/V173Y/A311G/K336S/K340W/A388K/ | +++ |
| | | V401R/Y404L/S437P/L514S/E659R/D660H/K720A/ | |
| | | H779R | |
| 263/264 | D196C | R38G/N138R/V173Y/D196C/A311G/K336S/K340W/ | +++ |
| | | A388K/V401R/Y404L/S437P/L514S/E659R/D660H/ | |
| | | H779R | |
| 265/266 | T609W | R38G/N138R/V173Y/A311G/K336S/K340W/A388K/ | +++ |
| | | V401R/Y404L/S437P/L514S/T609W/E659R/D660H/ | |
| | | H779R | |
| 267/268 | D196V | R38G/N138R/V173Y/D196V/A311G/K336S/K340W/ | +++ |
| | | A388K/V401R/Y404L/S437P/L514S/E659R/D660H/ | |
| | | H779R | |
| 269/270 | Q246V | R38G/N138R/V173Y/Q246V/A311G/K336S/K340W/ | +++ |
| | | A388K/V401R/Y404L/S437P/L514S/E659R/D660H/ | |
| | | H779R | |
| 271/272 | Y185L | R38G/N138R/V173Y/Y185L/A311G/K336S/K340W/ | +++ |
| | | A388K/V401R/Y404L/S437P/L514S/E659R/D660H/ | |
| | | H779R | |
| 273/274 | I594G | R38G/N138R/V173Y/A311G/K336S/K340W/A388K/ | +++ |
| | | V401R/Y404L/S437P/L514S/I594G/E659R/D660H/ | |
| | | H779R | |
| 275/276 | H212Y | R38G/N138R/V173Y/H212Y/A311G/K336S/K340W/ | +++ |
| | | A388K/V401R/Y404L/S437P/L514S/E659R/D660H/ | |
| | | H779R | |
| 277/278 | E494G | R38G/N138R/V173Y/A311G/K336S/K340W/A388K/ | +++ |
| | | V401R/Y404L/S437P/E494G/L514S/E659R/D660H/ | |
| | | H779R | |
| 279/280 | D196A | R38G/N138R/V173Y/D196A/A311G/K336S/K340W/ | +++ |
| | | A388K/V401R/Y404L/S437P/L514S/E659R/D660H/ | |
| | | H779R | |
| 281/282 | H212R | R38G/N138R/V173Y/H212R/A311G/K336S/K340W/ | +++ |
| | | A388K/V401R/Y404L/S437P/L514S/E659R/D660H/ | |
| | | H779R | |
| 283/284 | D375F | R38G/N138R/V173Y/A311G/K336S/K340W/D375F/ | +++ |
| | | A388K/V401R/Y404L/S437P/L514S/E659R/D660H/ | |
| | | H779R | |
| 285/286 | K721T | R38G/N138R/V173Y/A311G/K336S/K340W/A388K/ | ++ |
| | | V401R/Y404L/S437P/L514S/E659R/D660H/K721T/ | |
| | | H779R | |
| 287/288 | K721A | R38G/N138R/V173Y/A311G/K336S/K340W/A388K/ | ++ |
| | | V401R/Y404L/S437P/L514S/E659R/D660H/K721A/ | |
| | | H779R | |
| 289/290 | A72R | R38G/A72R/N138R/V173Y/A311G/K336S/K340W/ | ++ |
| | | A388K/V401R/Y404L/S437P/L514S/E659R/D660H/ | |
| | | H779R | |
| 291/292 | Y185I | R38G/N138R/V173Y/Y185I/A311G/K336S/K340W/ | ++ |
| | | A388K/V401R/Y404L/S437P/L514S/E659R/D660H/ | |
| | | H779R | |
| 293/294 | K721S | R38G/N138R/V173Y/A311G/K336S/K340W/A388K/ | ++ |
| | | V401R/Y404L/S437P/L514S/E659R/D660H/K721S/ | |
| | | H779R | |
| 295/296 | K867Q | R38G/N138R/V173Y/A311G/K336S/K340W/A388K/ | ++ |
| | | V401R/Y404L/S437P/L514S/E659R/D660H/H779R/ | |
| | | K867Q | |
| 297/298 | K721Y | R38G/N138R/V173Y/A311G/K336S/K340W/A388K/ | ++ |
| | | V401R/Y404L/S437P/L514S/E659R/D660H/K721Y/ | |
| | | H779R | |
| 299/300 | R160F | R38G/N138R/R160F/V173Y/A311G/K336S/K340W/ | ++ |
| | | A388K/V401R/Y404L/S437P/L514S/E659R/D660H/ | |
| | | H779R | |
| 301/302 | K718F | R38G/N138R/V173Y/A311G/K336S/K340W/A388K/ | ++ |
| | | V401R/Y404L/S437P/L514S/E659R/D660H/K718F/ | |
| | | H779R | |
| 303/304 | T609F | R38G/N138R/V173Y/A311G/K336S/K340W/A388K/ | + |
| | | V401R/Y404L/S437P/L514S/T609F/E659R/D660H/ | |
| | | H779R | |
| 305/306 | K720S | R38G/N138R/V173Y/A311G/K336S/K340W/A388K/ | + |
| | | V401R/Y404L/S437P/L514S/E659R/D660H/K720S/ | |
| | | H779R | |
| 307/308 | K187L | R38G/N138R/V173Y/K187L/A311G/K336S/K340W/ | + |
| | | A388K/V401R/Y404L/S437P/L514S/E659R/D660H/ | |
| | | H779R | |
| 309/310 | K718V | R38G/N138R/V173Y/A311G/K336S/K340W/A388K/ | + |
| | | V401R/Y404L/S437P/L514S/E659R/D660H/K718VH | |
| | | H779R | |
| 311/312 | K170L/Y173V | R38G/N138R/K170L/A311G/K336S/K340W/A388K/ | + |
| | | V401R/Y404L/S437P/L514S/E659R/D660H/H779R | |
| 313/314 | T609E | R38G/N138R/V173Y/A311G/K336S/K340W/A388K/ | + |
| | | V401R/Y404L/S437P/L514S/T609E/E659R/D660H/ | |
| | | H779R | |
| 315/316 | K718S | R38G/N138R/V173Y/A311G/K336S/K340W/A388K/ | + |
| | | V401R/Y404L/S437P/L514S/E659R/D660H/K718S/ | |
| | | H779R | |
| 317/318 | T609C | R38G/N138R/V173Y/A311G/K336S/K340W/A388K/ | + |
| | | V401R/Y404L/S437P/L514S/T609C/E659R/D660H/ | |
| | | H779R | |
| 319/320 | T609P | R38G/N138R/V173Y/A311G/K336S/K340W/A388K/ | + |
| | | V401R/Y404L/S437P/L514S/T609P/E659R/D660H/ | |
| | | H779R | |
| 321/322 | K721M | R38G/N138R/V173Y/A311G/K336S/K340W/A388K/ | + |
| | | V401R/Y404L/S437P/L514S/E659R/D660H/K721M/ | |
| | | H779R | |
| 323/324 | K615A | R38G/N138R/V173Y/A311G/K336S/K340W/A388K/ | + |
| | | V401R/Y404L/S437P/L514S/K615A/E659R/D660H/ | |
| | | H779R | |
| 325/326 | K187H | R38G/N138R/V173Y/K187H/A311G/K336S/K340W/ | + |
| | | A388K/V401R/Y404L/S437P/L514S/E659R/D660H/ | |
| | | H779R | |
| 327/328 | D196S | R38G/N138R/V173Y/D196S/A311G/K336S/K340W/ | + |
| | | A388K/V401R/Y404L/S437P/L514S/E659R/D660H/ | |
| | | H779R | |
| 329/330 | K721E | R38G/N138R/V173Y/A311G/K336S/K340W/A388K/ | + |
| | | V401R/Y404L/S437P/L514S/E659R/D660H/K721E/ | |
| | | H779R | |
| 331/332 | K718Y | R38G/N138R/V173Y/A311G/K336S/K340W/A388K/ | + |
| | | V401R/Y404L/S437P/L514S/E659R/D660H/K718Y/ | |
| | | H779R | |
| 333/334 | D137G/R138N | R38G/D137G/V173Y/A311G/K336S/K340W/A388K/ | + |
| | | V401R/Y404L/S437P/L514S/E659R/D660H/H779R | |
| 335/336 | K187G | R38G/N138R/V173Y/K187G/A311G/K336S/K340W/ | + |
| | | A388K/V401R/Y404L/S437P/L514S/E659R/D660H/ | |
| | | H779R | |
| 337/338 | K720G | R38G/N138R/V173Y/A311G/K336S/K340W/A388K/ | + |
| | | V401R/Y404L/S437P/L514S/E659R/D660H/K720G/ | |
| | | H779R | |
| 339/340 | K720P | R38G/N138R/V173Y/A311G/K336S/K340W/A388K/ | + |
| | | V401R/Y404L/S437P/L514S/E659R/D660H/K720P/ | |
| | | H779R | |
| 341/342 | D854T | R38G/N138R/V173Y/A311G/K336S/K340W/A388K/ | + |
| | | V401R/Y404L/S437P/L514S/E659R/D660H/H779R/ | |
| | | D854T | |
| 343/344 | K720I | R38G/N138R/V173Y/A311G/K336S/K340W/A388K/ | + |
| | | V401R/Y404L/S437P/L514S/E659R/D660H/K720I/ | |
| | | H779R | |
| 345/346 | R160L | R38G/N138R/R160L/V173Y/A311G/K336S/K340W/ | + |
| | | A388K/V401R/Y404L/S437P/L514S/E659R/D660H/ | |
| | | H779R | |
| 347/348 | A131T | R38G/A131T/N138R/V173Y/A311G/K336S/K340W/ | + |
| | | A388K/V401R/Y404L/S437P/L514S/E659R/D660H/ | |
| | | H779R | |
| 349/350 | G38R/Y185L/K187H/ | N138R/V173Y/Y185L/K187H/A311G/K336S/K340W/ | ++ |
| | P437S | A388K/V401R/Y404L/L514S/E659R/D660H/H779R | |
| 351/352 | Y185L/K187H | R38G/N138R/V173Y/Y185L/K187H/A311G/K336S/ | ++ |
| | | K340W/A388K/V401R/Y404L/S437P/L514S/E659R/ | |
| | | D660H/H779R | |
| 353/354 | Y185L/K187L | R38G/N138R/V173Y/Y185L/K187L/A311G/K336S/ | ++ |
| | | K340W/A388K/V401R/Y404L/S437P/L514S/E659R/ | |
| | | D660H/H779R | |
| 355/356 | P437S/T609C | R38G/N138R/V173Y/A311G/K336S/K340W/A388K/ | ++ |
| | | V401R/Y404L/L514S/T609C/E659R/D660H/H779R | |
| 357/358 | G38R/T609P | N138R/V173Y/A311G/K336S/K340W/A388K/V401R/ | ++ |
| | | Y404L/S437P/L514S/T609P/E659R/D660H/H779R | |
| 359/360 | G38R/Y185L/K187H/ | N138R/V173Y/Y185L/K187H/A311G/K336S/K340W/ | ++ |
| | P437S/T609C | A388K/V401R/Y404L/L514S/T609C/E659R/D660H/ | |
| | | H779R | |
| 361/362 | P437S/T609P/K720A | R38G/N138R/V173Y/A311G/K336S/K340W/A388K/ | ++ |
| | | V401R/Y404L/L514S/T609P/E659R/D660H/K720A/ | |
| | | H779R | |
| 363/364 | Y185L/K187L/T609P | R38G/N138R/V173Y/Y185L/K187L/A311G/K336S/ | ++ |
| | | K340W/A388K/V401R/Y404L/S437P/L514S/T609P/ | |
| | | E659R/D660H/H779R | |
| 365/366 | Y185L/K187L/P437S | R38G/N138R/V173Y/Y185L/K187L/A311G/K336S/ | ++ |
| | | K340W/A388K/V401R/Y404L/L514S/E659R/D660H/ | |
| | | H779R | |
| 367/368 | Y185L/K187H/P437S | R38G/N138R/V173Y/Y185L/K187H/A311G/K336S/ | + |
| | | K340W/A388K/V401R/Y404L/L514S/E659R/D660H/ | |
| | | H779R | |
| 369/370 | P437S/T609P | R38G/N138R/V173Y/A311G/K336S/K340W/A388K/ | + |
| | | V401R/Y404L/L514S/T609P/E659R/D660H/H779R | |
| 371/372 | P437S/K721S | R38G/N138R/V173Y/A311G/K336S/K340W/A388K/ | + |
| | | V401R/Y404L/L514S/E659R/D660H/K721S/H779R | |
| 373/374 | Y185L/K187H/T609E | R38G/N138R/V173Y/Y185L/K187H/A311G/K336S/ | + |
| | | K340W/A388K/V401R/Y404L/S437P/L514S/T609E/ | |
| | | E659R/D660H/H779R | |
| 375/376 | T609P/K720A | R38G/N138R/V173Y/A311G/K336S/K340W/A388K/ | + |
| | | V401R/Y404L/S437P/L514S/T609P/E659R/D660H/ | |
| | | K720A/H779R | |
| 377/378 | Y185L/K187L/Q246V | R38G/N138R/V173Y/Y185L/K187L/Q246V/A311G/ | + |
| | | K336S/K340W/A388K/V401R/Y404L/S437P/L514S/ | |
| | | 5E69R/D660H/H779R | |
| 379/380 | Y185L/K187H/P437S/ | R38G/N138R/V173Y/Y185L/K187H/A311G/K336S/ | + |
| | T609P/K720A | K340W/A388K/V401R/Y404L/L514S/T609P/E659R/ | |
| | | D660H/K720A/H779R | |

Levels of increased activity were determined relative to the reference polypeptide of SEQ ID NO: 234 and defined as follows: ″+″ 1.92 to 11.20 (first 50%), ″++″ >11.20 (next 30%), ″+++″ >22.95 (top 20%)

### Example 12

### Improvements Over RNA Polymerase of SEQ ID NO: 156

SEQ ID NO: 156 was selected as the parent enzyme in the in vitro transcription (IVT) assay. SEQ ID NO: 381 and SEQ ID NO: 383 were rationally designed, de novo synthesized, and cloned into the expression vector pCK110900 (See, FIG. 3 of US Pat. Appln. Publn. No. 2006/0195947) operatively linked to the lac promoter under control of the lac1 repressor. The expression vector also contains the P15a origin of replication and the chloramphenicol resistance gene. The resulting plasmids were transformed into E. coli BL21, using standard methods known in the art. The transformants were isolated by subjecting the cells to chloramphenicol selection, as known in the art (See e.g., U.S. Pat. No. 8,383,346 and WO2010/144103). The polypeptides encoded by each gene were generated and purified from the shake flask cultures as described in Example 4. Each variant was assayed in a 1 μL reaction that comprised 0.05 μg/μL 11 kb-DNA template, 40 mM Tris-HCl pH 8.0, 42 mM magnesium acetate, 10 mM ATP, 10 mM GTP, 10 mM CTP, 10 mM UTP, 1 U/μL Ribolock RNase Inhibitor (Thermo Scientific, Catalog #E00382), 0.002 U/μL Pyrophosphatase (Thermo Scientific, Catalog #EF0221), and a final concentration of 20 ng/μL of variants. The IVT reactions were initiated by adding the variants, incubated in Bio-Rad Hard-Shell® 96-Well PCR plates at 25° C. for 2 h, and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 156 (Activity FIOP) was calculated as the RNA produced by variant over the RNA produced by SEQ ID NO: 156. The result is shown in Table 12.1.

TABLE 12.1
Activities of Variants Relative to SEQ ID NO: 156

| | | | FIOP Product |
|---|---|---|---|
| SEQ ID | | | Relative to |
| NO: | Amino Acid Differences | Amino Acid Differences | SEQ ID NO: |
| (nt/aa) | (Relative to SEQ ID NO: 156) | (Relative to SEQ ID NO: 2) | 156 |
| 381/382 | R357E/R394K/W664P | N138R/V173Y/A311G/K336S/K340W/R357E/ | + |
| | | A388K/R394K/V401R/Y404L/L514S/E659R/ | |
| | | D660H/W664P | |
| 383/384 | R138N/G311A/S336K/W340K/ | V173Y/R357E/R394K/V401R/Y404S/L514S/ | + |
| | R357E/K388A/R394K/L404S/ | E659R/D660H/W664P | |
| | W664P | | |

Levels of increased activity were determined relative to the reference polypeptide of SEQ ID NO: 156 and defined as follows: ″+″ from 1.09 to 1.22

### Example 13

### Improvements Over RNA Polymerase of SEQ ID NO: 386

Plasmid containing SEQ ID NO: 381 was transformed into E. coli W3110 to give SEQ ID NO: 385, which was selected as the parent enzyme in the in vitro transcription (IV) assay. Unlike SEQ ID NO: 381 expressed in E. coli BL21 and rendering functional polypeptides, SEQ ID NO: 385 in E. coli W3110 was not expressed. To achieve soluble and functional expression, libraries of engineered genes were produced using known techniques (e.g., saturation mutagenesis, recombination of previously identified beneficial mutations). The polypeptides encoded by each gene were produced in HTP as described in Example 2 and purified as described in Example 3. Each variant was diluted 4-fold in RNase-free water and screened in a 1 μL reaction that comprised 0.05 μg/μL 11 kb-DNA template, 40 mM Tris-HCl pH 8.0, 42 mM magnesium acetate, 10 mM ATP, 10 mM GTP, 10 mM CTP, 10 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HTP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). As SEQ ID NO: 386 failed to be functionally expressed in E. coli W3110, RNA produced by SEQ ID NO: 386 was much smaller than 1 ng/μL and therefore rounded up to 1 ng/μL. Activity relative to SEQ ID NO: 386 (Activity FIOP) was calculated as the RNA produced by variant over the RNA produced by SEQ ID NO: 386. The result is shown in Table 13.1.

TABLE 13.1
Activities of Variants Relative to SEQ ID: 386

| | | | FIOP Product |
|---|---|---|---|
| SEQ ID | | | Relative to |
| NO: | Amino Acid Differences | Amino Acid Differences | SEQ ID NO: |
| (nt/aa) | (Relative to SEQ ID NO: 386) | (Relative to SEQ ID NO: 2) | 386 |
| 385/386 | — | N138R/V173Y/A311G/K336S/K340W/R357E/A388K/ | — |
| | | R394K/V401R/Y404L/L514S/E659R/D660H/ | |
| | | W664P | |
| 387/388 | K170L/T609P | N138R/K170L/V173Y/A311G/K336S/K340W/ | +++ |
| | | R357E/A388K/R394K/V401R/Y404L/L514S/T609P/ | |
| | | E659R/D660H/W664P | |
| 389/390 | R38G/Y185L/K187L/T609C | R38G/N138R/V173Y/Y185L/K187L/A311G/K336S/ | +++ |
| | | K340W/R357E/A388K/R394K/V401R/Y404L/ | |
| | | L514S/T609C/E659R/D660H/W664P | |
| 391/392 | R38G/Y185L/K187H | R38G/N138R/V173Y/Y185L/K187H/A311G/K336S/ | +++ |
| | | K340W/R357E/A388K/R394K/V401R/Y404L/ | |
| | | L514S/E659R/D660H/W664P | |
| 393/394 | R38G/K170L/Y173V | R38G/N138R/K170L/A311G/K336S/K340W/R357E/ | +++ |
| | | A388K/R394K/V401R/Y404L/L514S/E659R/ | |
| | | D660H/W664P | |
| 395/396 | R38G/Y173V/Y185L/ | R38G/N138R/Y185L/K187L/A311G/K336S/K340W/ | +++ |
| | K187L/K720A | R357E/A388K/R394K/V401R/Y404L/L514S/E659R/ | |
| | | D660H/W664P/K720A | |
| 397/398 | K170L/Y185L/K187H/ | N138R/K170L/V173Y/Y185L/K187H/A311G/K336S/ | +++ |
| | T609C | K340W/R357E/A388K/R394K/V401R/Y404L/L514S/ | |
| | | T609C/E659R/D660H/W664P | |
| 399/400 | T609E | N138R/V173Y/A311G/K336S/K340W/R357E/A388K/ | +++ |
| | | R394K/V401R/Y404L/L514S/T609E/E659R/D660H/ | |
| | | W664P | |
| 401/402 | K170L/Y185L/K187H/ | N138R/K170L/V173Y/Y185L/K187H/A311G/K336S/ | ++ |
| | T609C/K721S | K340W/R357E/A388K/R394K/V401R/Y404L/ | |
| | | L514S/T609C/E659R/D660H/W664P/K721S | |
| 403/404 | R38G/T609E | R38G/N138R/V173Y/A311G/K336S/K340W/R357E/ | ++ |
| | | A388K/R394K/V401R/Y404L/L514S/T609E/E659R/ | |
| | | D660H/W664P | |
| 405/406 | K170L/Y185L/K187L/ | N138R/K170L/V173Y/Y185L/K187L/A311G/K336S/ | ++ |
| | T609E | K340W/R357E/A388K/R394K/V401R/Y404L/L514S/ | |
| | | T609E/E659R/D660H/W664P | |
| 407/408 | R38G/K170L/Q246V/ | R38G/N138R/K170L/V173Y/Q246V/A311G/K336S/ | ++ |
| | T609P | K340W/R357E/A388K/R394K/V401R/Y404L/L514S/ | |
| | | T609P/E659R/D660H/W664P | |
| 409/410 | Y185L/K187H/T609E/ | N138R/V173Y/Y185L/K187H/A311G/K336S/K340W/ | ++ |
| | K718S | R357E/A388K/R394K/V401R/Y404L/L514S/T609E/ | |
| | | E659R/D660H/W664P/K718S | |
| 411/412 | R38G/Y185L/K187L/ | R38G/N138R/V173Y/Y185L/K187L/A311G/K336S/ | ++ |
| | T609W/K718N/K720A | K340W/R357E/A388K/R394K/V401R/Y404L/L514S/ | |
| | | T609W/E659R/D660H/W664P/K718N/K720A | |
| 413/414 | R38G/K170L/Q246V/ | R38G/N138R/K170L/V173Y/Q246V/A311G/K336S/ | ++ |
| | T609C/K718S | K340W/R357E/A388K/R394K/V401R/Y404L/L514S/ | |
| | | T609C/E659R/D660H/W664P/K718S | |
| 415/416 | K720A | N138R/V173Y/A311G/K336S/K340W/R357E/A388K/ | ++ |
| | | R394K/V401R/Y404L/L514S/E659R/D660H/W664P/ | |
| | | K720A | |
| 417/418 | Q246V/T609E | N138R/V173Y/Q246V/A311G/K336S/K340W/R357E/ | ++ |
| | | A388K/R394K/V401R/Y404L/L514S/T609E/E659R/ | |
| | | D660H/W664P | |
| 419/420 | R38G/Y185L/K187L/ | R38G/N138R/V173Y/Y185L/K187L/Q246V/A311G/ | ++ |
| | Q246V/K720A | K336S/K340W/R357E/A388K/R394K/V401R/Y404L/ | |
| | | L514S/E659R/D660H/W664P/K720A | |
| 421/422 | R38G/T609C/K721S | R38G/N138R/V173Y/A311G/K336S/K340W/R357E/ | ++ |
| | | A388K/R394K/V401R/Y404L/L514S/T609C/E659R/ | |
| | | D660H/W664P/K721S | |
| 423/424 | R38G/K721S | R38G/N138R/V173Y/A311G/K336S/K340W/R357E/ | + |
| | | A388K/R394K/V401R/Y404L/L514S/E659R/D660H/ | |
| | | W664P/K721S | |
| 425/426 | R38G/K720A/K721S | R38G/N138R/V173Y/A311G/K336S/K340W/R357E/ | + |
| | | A388K/R394K/V401R/Y404L/L514S/E659R/D660H/ | |
| | | W664P/K720A/K721S | |
| 427/428 | R38G/K170L/T609C/ | R38G/N138R/K170L/V173Y/A311G/K336S/K340W/ | + |
| | K718S/K720A | R357E/A388K/R394K/V401R/Y404L/L514S/T609C/ | |
| | | E659R/D660H/W664P/K718S/K720A | |
| 429/430 | R38G/K718S | R38G/N138R/V173Y/A311G/K336S/K340W/R357E/ | + |
| | | A388K/R394K/V401R/Y404L/L514S/E659R/D660H/ | |
| | | W664P/K718S | |
| 431/432 | R38G/K170L/Q246V/ | R38G/N138R/K170L/V173Y/Q246V/A311G/K336S/ | + |
| | T609P/K718S | K340W/R357E/A388K/R394K/V401R/Y404L/L514S/ | |
| | | T609P/E659R/D660H/W664P/K718S | |
| 433/434 | R38G/Y185L/K187L/ | R38G/N138R/V173Y/Y185L/K187L/A311G/K336S/ | + |
| | T609P/K718S/K721S | K340W/R357E/A388K/R394K/V401R/Y404L/L514S/ | |
| | | T609P/E659R/D660H/W664P/K718S/K721S | |
| 435/436 | Q529R/T609E | N138R/V173Y/A311G/K336S/K340W/R357E/A388K/ | + |
| | | R394K/V401R/Y404L/L514S/Q529R/T609E/E659R/ | |
| | | D660H/W664P | |
| 437/438 | R38G/T609P/K718S | R38G/N138R/V173Y/A311G/K336S/K340W/R357E/ | + |
| | | A388K/R394K/V401R/Y404L/L514S/T609P/E659R/ | |
| | | D660H/W664P/K718S | |
| 439/440 | Y185L/K187L/T609C/ | N138R/V173Y/Y185L/K187L/A311G/K336S/K340W/ | + |
| | K718S/K720A | R357E/A388K/R394K/V401R/Y404L/L514S/T609C/ | |
| | | E659R/D660H/W664P/K718S/K720A | |
| 441/442 | Y185L/K187H/T609C/ | N138R/V173Y/Y185L/K187H/A311G/K336S/K340W/ | + |
| | K718S/K721S | R357E/A388K/R394K/V401R/Y404L/L514S/T609C/ | |
| | | E659R/D660H/W664P/K718S/K721S | |
| 443/444 | R38G/K170L/K718S/ | R38G/N138R/K170L/V173Y/A311G/K336S/K340W/ | + |
| | K720A/K721S | R357E/A388K/R394K/V401R/Y404L/L514S/E659R/ | |
| | | D660H/W664P/K718S/K720A/K721S | |
| 445/446 | K170L/Y185L/K187L/ | N138R/K170L/V173Y/Y185L/K187L/A311G/K336S/ | + |
| | K718S/K720A | K340W/R357E/A388K/R394K/V401R/Y404L/L514S/ | |
| | | E659R/D660H/W664P/K718S/K720A | |
| 447/448 | R38G/Y173V/Y185L/ | R38G/N138R/Y185L/K187L/Q246V/A311G/K336S/ | + |
| | K187L/Q246V/T609E/ | K340W/R357E/A388K/R394K/V401R/Y404L/L514S/ | |
| | K720A | T609E/E659R/D660H/W664P/K720A | |
| 449/450 | T609E/K718S/K720A | N138R/V173Y/A311G/K336S/K340W/R357E/A388K/ | + |
| | | R394K/V401R/Y404L/L514S/T609E/E659R/D660H/ | |
| | | W664P/K718S/K720A | |
| 451/452 | K170L/Y185L/K187H/ | N138R/K170L/V173Y/Y185L/K187H/A311G/K336S/ | + |
| | K718S/K720A/K721S | K340W/R357E/A388K/R394K/V401R/Y404L/L514S/ | |
| | | E659R/D660H/W664P/K718S/K720A/K721S | |
| 453/454 | R38G/K170L/Y173V/ | R38G/N138R/K170L/Q246V/A311G/K336S/K340W/ | + |
| | Q246V/T609E/K718S | R357E/A388K/R394K/V401R/Y404L/L514S/T609E/ | |
| | | E659R/D660H/W664P/K718S | |
| 455/456 | Q246V/K721S | N138R/V173Y/Q246V/A311G/K336S/K340W/R357E/ | + |
| | | A388K/R394K/V401R/Y404L/L514S/E659R/D660H/ | |
| | | W664P/K721S | |
| 457/458 | R38G/K718S/K720A | R38G/N138R/V173Y/A311G/K336S/K340W/R357E/ | + |
| | | A388K/R394K/V401R/Y404L/L514S/E659R/D660H/ | |
| | | W664P/K718S/K720A | |
| 459/460 | R38G/T609E/K718S/ | R38G/N138R/V173Y/A311G/K336S/K340W/R357E/ | + |
| | K720A | A388K/R394K/V401R/Y404L/L514S/T609E/E659R/ | |
| | | D660H/W664P/K718S/K720A | |

Levels of increased activity were determined relative to the reference polypeptide of SEQ ID NO: 386 and defined as follows: ″+″ 4.11 to 10.95 (first 50%), ″++″ >10.95 (next 30%), ″+++″ >34.67 (top 20%)

### Example 14

### Improvements Over RNA Polymerase of SEQ ID NO: 238

SEQ ID NO: 238 was selected as the parent enzyme in the in vitro transcription (IV) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 11 kb-DNA template, 40 mM Tris-HCl pH 8.0, 42 mM magnesium acetate, 10 mM ATP, 10 mM GTP, 10 mM CTP, 10 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 238 (Activity FIOP) was calculated as the RNA produced by variant over the RNA produced by SEQ ID NO: 238. The result is shown in Table 14.1.

TABLE 14.1
Activities of Variants Relative to SEQ ID: 238

| | Amino Acid | Amino Acid | |
|---|---|---|---|
| SEQ ID | Differences | Differences | FIOP Product |
| NO: | (Relative to | (Relative to | Relative to SEQ |
| (nt/aa) | SEQ ID NO: 238) | SEQ ID NO: 2) | ID NO: 238 |
| 461/462 | A143N | R38G/N138R/A143N/V173Y/A311G/K336S/ | +++ |
| | | K340W/A388K/V401R/Y404L/L514S/E659R/ | |
| | | D660H | |
| 463/464 | A143Q | R38G/N138R/A143Q/V173Y/A311G/K336S/ | ++ |
| | | K340W/A388K/V401R/Y404L/L514S/E659R/ | |
| | | D660H | |
| 465/466 | A143R | R38G/N138R/A143R/V173Y/A311G/K336S/ | ++ |
| | | K340W/A388K/V401R/Y404L/L514S/E659R/ | |
| | | D660H | |
| 467/468 | K105R | R38G/K105R/N138R/V173Y/A311G/K336S/ | + |
| | | K340W/A388K/V401R/Y404L/L514S/E659R/ | |
| | | D660H | |
| 469/470 | E775R/H779R | R38G/N138R/V173Y/A311G/K336S/K340W/ | + |
| | | A388K/V401R/Y404L/L514S/E659R/D660H/ | |
| | | E775R/H779R | |
| 471/472 | S135K | R38G/S135K/N138R/V173Y/A311G/K336S/ | + |
| | | K340W/A388K/V401R/Y404L/L514S/E659R/ | |
| | | D660H | |
| 473/474 | K100R | R38G/K100R/N138R/V173Y/A311G/K336S/ | + |
| | | K340W/A388K/V401R/Y404L/L514S/E659R/ | |
| | | D660H | |
| 475/476 | E775K/H779R | R38G/N138R/V173Y/A311G/K336S/K340W/ | + |
| | | A388K/V401R/Y404L/L514S/E659R/D660H/ | |
| | | E775K/H779R | |
| 477/478 | Q246K | R38G/N138R/V173Y/Q246K/A311G/K336S/ | + |
| | | K340W/A388K/V401R/Y404L/L514S/E659R/ | |
| | | D660H | |
| 479/480 | H779R/E782R | R38G/N138R/V173Y/A311G/K336S/K340W/ | + |
| | | A388K/V401R/Y404L/L514S/E659R/D660H/ | |
| | | H779R/E782R | |
| 481/482 | P749R | R38G/N138R/V173Y/A311G/K336S/K340W/ | + |
| | | A388K/V401R/Y404L/L514S/E659R/D660H/ | |
| | | P749R | |
| 483/484 | H237L | R38G/N138R/V173Y/H237L/A311G/K336S/ | +++ |
| | | K340W/A388K/V401R/Y404L/L514S/E659R/ | |
| | | D660H | |
| 485/486 | R162V | R38G/N138R/R162V/V173Y/A311G/K336S/ | +++ |
| | | K340W/A388K/V401R/Y404L/L514S/E659R/ | |
| | | D660H | |
| 487/488 | T83K | R38G/T83K/N138R/V173Y/A311G/K336S/ | +++ |
| | | K340W/A388K/V401R/Y404L/L514S/E659R/ | |
| | | D660H | |
| 489/490 | H218T | R38G/N138R/V173Y/H218T/A311G/K336S/ | +++ |
| | | K340W/A388K/V401R/Y404L/L514S/E659R/ | |
| | | D660H | |
| 491/492 | L227V | R38G/N138R/V173Y/L227V/A311G/K336S/ | +++ |
| | | K340W/A388K/V401R/Y404L/L514S/E659R/ | |
| | | D660H | |
| 493/494 | E115L | R38G/E115L/N138R/V173Y/A311G/K336S/ | +++ |
| | | K340W/A388K/V401R/Y404L/L514S/E659R/ | |
| | | D660H | |
| 495/496 | M274G | R38G/N138R/V173Y/M274G/A311G/K336S/ | +++ |
| | | K340W/A388K/V401R/Y404L/L514S/E659R/ | |
| | | D660H | |
| 497/498 | I81C | R38G/I81C/N138R/V173Y/A311G/K336S/ | +++ |
| | | K340W/A388K/V401R/Y404L/L514S/E659R/ | |
| | | D660H | |
| 499/500 | A122I | R38G/A122I/N138R/V173Y/A311G/K336S/ | +++ |
| | | K340W/A388K/V401R/Y404L/L514S/E659R/ | |
| | | D660H | |
| 501/502 | M61Y | R38G/M61Y/N138R/V173Y/A311G/K336S/ | +++ |
| | | K340W/A388K/V401R/Y404L/L514S/E659R/ | |
| | | D660H | |
| 503/504 | A32S | A32S/R38G/N138R/V173Y/A311G/K336S/ | +++ |
| | | K340W/A388K/V401R/Y404L/L514S/E659R/ | |
| | | D660H | |
| 505/506 | T139R | R38G/N138R/T139R/V173Y/A311G/K336S/ | ++ |
| | | K340W/A388K/V401R/Y404L/L514S/E659R/ | |
| | | D660H | |
| 507/508 | A75Y | R38G/A75Y/N138R/V173Y/A311G/K336S/ | ++ |
| | | K340W/A388K/V401R/Y404L/L514S/E659R/ | |
| | | D660H | |
| 509/510 | Q239R | R38G/N138R/V173Y/Q239R/A311G/K336S/ | ++ |
| | | K340W/A388K/V401R/Y404L/L514S/E659R/ | |
| | | D660H | |
| 511/512 | Y257L | R38G/N138R/V173Y/Y257L/A311G/K336S/ | ++ |
| | | K340W/A388K/V401R/Y404L/L514S/E659R/ | |
| | | D660H | |
| 513/514 | N240M | R38G/N138R/V173Y/N240M/A311G/K336S/ | ++ |
| | | K340W/A388K/V401R/Y404L/L514S/E659R/ | |
| | | D660H | |
| 515/516 | G69S/I161R | R38G/G69S/N138R/I161R/V173Y/A311G/ | ++ |
| | | K336S/K340W/A388K/V401R/Y404L/L514S/ | |
| | | E659R/D660H | |
| 517/518 | L253Y | R38G/N138R/V173Y/L253Y/A311G/K336S/ | ++ |
| | | K340W/A388K/V401R/Y404L/L514S/E659R/ | |
| | | D660H | |
| 519/520 | A14Y | A14Y/R38G/N138R/V173Y/A311G/K336S/ | ++ |
| | | K340W/A388K/V401R/Y404L/L514S/E659R/ | |
| | | D660H | |
| 521/522 | V221C | R38G/N138R/V173Y/V221C/A311G/K336S/ | ++ |
| | | K340W/A388K/V401R/Y404L/L514S/E659R/ | |
| | | D660H | |
| 523/524 | L84A | R38G/L84A/N138R/V173Y/A311G/K336S/ | ++ |
| | | K340W/A388K/V401R/Y404L/L514S/E659R/ | |
| | | D660H | |
| 525/526 | E115V | R38G/E115V/N138R/V173Y/A311G/K336S/ | ++ |
| | | K340W/A388K/V401R/Y404L/L514S/E659R/ | |
| | | D660H | |
| 527/528 | L133C/R150Q | R38G/L133C/N138R/R150Q/V173Y/A311G/ | ++ |
| | | K336S/K340W/A388K/V401R/Y404L/L514S/ | |
| | | E659R/D660H | |
| 529/530 | K60L | R38G/K60L/N138R/V173Y/A311G/K336S/ | ++ |
| | | K340W/A388K/V401R/Y404L/L514S/E659R/ | |
| | | D660H | |
| 531/532 | K60G | R38G/K60G/N138R/V173Y/A311G/K336S/ | ++ |
| | | K340W/A388K/V401R/Y404L/L514S/E659R/ | |
| | | D660H | |
| 533/534 | A32V | A32V/R38G/N138R/V173Y/A311G/K336S/ | ++ |
| | | K340W/A388K/V401R/Y404L/L514S/E659R/ | |
| | | D660H | |
| 535/536 | F275A | R38G/N138R/V173Y/F275A/A311G/K336S/ | ++ |
| | | K340W/A388K/V401R/Y404L/L514S/E659R/ | |
| | | D660H | |
| 537/538 | I89Q | R38G/I89Q/N138R/V173Y/A311G/K336S/ | ++ |
| | | K340W/A388K/V401R/Y404L/L514S/E659R/ | |
| | | D660H | |
| 539/540 | Y257V | R38G/N138R/V173Y/Y257V/A311G/K336S/ | + |
| | | K340W/A388K/V401R/Y404L/L514S/E659R/ | |
| | | D660H | |
| 541/542 | M274L | R38G/N138R/V173Y/M274L/A311G/K336S/ | + |
| | | K340W/A388K/V401R/Y404L/L514S/E659R/ | |
| | | D660H | |
| 543/544 | K15T | K15T/R38G/N138R/V173Y/A311G/K336S/ | + |
| | | K340W/A388K/V401R/Y404L/L514S/E659R/ | |
| | | D660H | |
| 545/546 | A254S | R38G/N138R/V173Y/A254S/A311G/K336S/ | + |
| | | K340W/A388K/V401R/Y404L/L514S/E659R/ | |
| | | D660H | |
| 547/548 | M61W | R38G/M61W/N138R/V173Y/A311G/K336S/ | + |
| | | K340W/A388K/V401R/Y404L/L514S/E659R/ | |
| | | D660H | |
| 549/550 | G625C | R38G/N138R/V173Y/A311G/K336S/K340W/ | + |
| | | A388K/V401R/Y404L/L514S/G625C/E659R/ | |
| | | D660H | |
| 551/552 | A14Q/K67R | A14Q/R38G/K67R/N138R/V173Y/A311G/ | + |
| | | K336S/K340W/A388K/V401R/Y404L/L514S/ | |
| | | E659R/D660H | |
| 553/554 | I21G | I21G/R38G/N138R/V173Y/A311G/K336S/ | + |
| | | K340W/A388K/V401R/Y404L/L514S/E659R/ | |
| | | D660H | |
| 555/556 | A254G | R38G/N138R/V173Y/A254G/A311G/K336S/ | + |
| | | K340W/A388K/V401R/Y404L/L514S/E659R/ | |
| | | D660H | |
| 557/558 | N93Y | R38G/N93Y/N138R/V173Y/A311G/K336S/ | + |
| | | K340W/A388K/V401R/Y404L/L514S/E659R/ | |
| | | D660H | |
| 559/560 | T250R | R38G/N138R/V173Y/T250R/A311G/K336S/ | + |
| | | K340W/A388K/V401R/Y404L/L514S/E659R/ | |
| | | D660H | |
| 561/562 | I217V | R38G/N138R/V173Y/I217V/A311G/K336S/ | + |
| | | K340W/A388K/V401R/Y404L/L514S/E659R/ | |
| | | D660H | |
| 563/564 | K60M | R38G/K60M/N138R/V173Y/A311G/K336S/ | + |
| | | K340W/A388K/V401R/Y404L/L514S/E659R/ | |
| | | D660H | |
| 565/566 | Q239L | R38G/N138R/V173Y/Q239L/A311G/K336S/ | + |
| | | K340W/A388K/V401R/Y404L/L514S/E659R/ | |
| | | D660H | |
| 567/568 | A75S | R38G/A75S/N138R/V173Y/A311G/K336S/ | + |
| | | K340W/A388K/V401R/Y404L/L514S/E659R/ | |
| | | D660H | |
| 569/570 | A32T | A32T/R38G/N138R/V173Y/A311G/K336S/ | + |
| | | K340W/A388K/V401R/Y404L/L514S/E659R/ | |
| | | D660H | |
| 571/572 | S19E | S19E/R38G/N138R/V173Y/A311G/K336S/ | + |
| | | K340W/A388K/V401R/Y404L/L514S/E659R/ | |
| | | D660H | |
| 573/574 | W95Y | R38G/W95Y/N138R/V173Y/A311G/K336S/ | + |
| | | K340W/A388K/V401R/Y404L/L514S/E659R/ | |
| | | D660H | |
| 575/576 | I81L | R38G/I81L/N138R/V173Y/A311G/K336S/ | + |
| | | K340W/A388K/V401R/Y404L/L514S/E659R/ | |
| | | D660H | |
| 577/578 | K60R | R38G/K60R/N138R/V173Y/A311G/K336S/ | + |
| | | K340W/A388K/V401R/Y404L/L514S/E659R/ | |
| | | D660H | |
| 579/580 | L759M | R38G/N138R/V173Y/A311G/K336S/K340W/ | + |
| | | A388K/V401R/Y404L/L514S/E659R/D660H/ | |
| | | L759M | |
| 581/582 | F62T | R38G/F62T/N138R/V173Y/A311G/K336S/ | + |
| | | K340W/A388K/V401R/Y404L/L514S/E659R/ | |
| | | D660H | |
| 583/584 | T140R | R38G/N138R/T140R/V173Y/A311G/K336S/ | + |
| | | K340W/A388K/V401R/Y404L/L514S/E659R/ | |
| | | D660H | |

Levels of increased activity were determined relative to the reference polypeptide of SEQ ID NO: 238 and defined as follows: “+” 1.00 to 1.45 (first 50%), “++” >1.45 (next 30%), “+++” >1.78 (top 20%)

### Example 15

### Improvements Over RNA Polymerase of SEQ ID NO: 394

SEQ ID NO: 394 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 11 kb-DNA template, 40 mM Tris-HCl pH 8.0, 42 mM magnesium acetate, 10 mM ATP, 10 mM GTP, 10 mM CTP, 10 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 394 (Activity FIOP) was calculated as the RNA produced by variant over the RNA produced by SEQ ID NO: 394. The result is shown in Table 15.1.

TABLE 15.1
Activities of Variants Relative to SEQ ID: 394

| | Amino Acid | Amino Acid | FIOP Product |
|---|---|---|---|
| SEQ ID | Differences | Differences | Relative to |
| NO: | (Relative to | (Relative to | SEQ ID NO: |
| (nt/aa) | SEQ ID NO: 394) | SEQ ID NO: 2) | 394 |
| 585/586 | T609E/H779R | R38G/N138R/K170L/A311G/K336S/K340W/R357E/ | +++ |
| | | A388K/R394K/V401R/Y404L/L514S/T609E/ | |
| | | E659R/D660H/W664P/H779R | |
| 587/588 | Q246K/T609E | R38G/N138R/K170L/Q246K/A311G/K336S/ | ++ |
| | | K340W/R357E/A388K/R394K/V401R/Y404L/L514S/ | |
| | | T609E/E659R/D660H/W664P | |
| 589/590 | Q246K | R38G/N138R/K170L/Q246K/A311G/K336S/K340W/ | ++ |
| | | R357E/A388K/R394K/V401R/Y404L/L514S/ | |
| | | E659R/D660H/W664P | |
| 591/592 | T609E/E775K/H779R | R38G/N138R/K170L/A311G/K336S/K340W/R357E/ | ++ |
| | | A388K/R394K/V401R/Y404L/L514S/T609E/ | |
| | | E659R/D660H/W664P/E775K/H779R | |
| 593/594 | K105R | R38G/K105R/N138R/K170L/A311G/K336S/K340W/ | + |
| | | R357E/A388K/R394K/V401R/Y404L/L514S/ | |
| | | E659R/D660H/W664P | |
| 595/596 | A143N/T609E/E775K | R38G/N138R/A143N/K170L/A311G/K336S/K340W/ | + |
| | | R357E/A388K/R394K/V401R/Y404L/L514S/ | |
| | | T609E/E659R/D660H/W664P/E775K | |
| 597/598 | K105R/S135K/T609E/ | R38G/K105R/S135K/N138R/K170L/A311G/K336S/ | + |
| | E775K | K340W/R357E/A388K/R394K/V401R/Y404L/L514S/ | |
| | | T609E/E659R/D660H/W664P/E775K | |
| 599/600 | Q111K/T609E | R38G/Q111K/N138R/K170L/A311G/K336S/K340W/ | + |
| | | R357E/A388K/R394K/V401R/Y404L/L514S/ | |
| | | T609E/E659R/D660H/W664P | |
| 601/602 | T609E | R38G/N138R/K170L/A311G/K336S/K340W/R357E/ | + |
| | | A388K/R394K/V401R/Y404L/L514S/T609E/ | |
| | | E659R/D660H/W664P | |

Levels of increased activity were determined relative to the reference polypeptide of SEQ ID NO: 394 and defined as follows: “+” 1.06 to 2.44 (first 50%), “++” >2.44 (next 30%), “+++” >3.37 (top 20%)

### Example 16

### Improvements Over RNA Polymerase of SEQ ID NO: 588

SEQ ID NO: 588 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 11 kb-DNA template, 40 mM Tris-HCl pH 8.0, 50 mM magnesium acetate, 12 mM ATP, 12 mM GTP, 12 mM CTP, 12 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 588 (Activity FIOP) was calculated as the RNA produced by variant over the RNA produced by SEQ ID NO: 588. The result is shown in Table 16.1.

TABLE 16.1
Activities of Variants Relative to SEQ ID NO: 588

| | Amino Acid | Amino Acid | FIOP Product |
|---|---|---|---|
| SEQ ID | Differences | Differences | Relative to |
| NO: | (Relative to | (Relative to | SEQ ID NO: |
| (nt/aa) | SEQ ID NO: 588) | SEQ ID NO: 2) | 588 |
| 603/604 | A32V | A32V/R38G/N138R/K170L/Q246K/A311G/K336S/ | +++ |
| | | K340W/R357E/A388K/R394K/V401R/Y404L/L514S/ | |
| | | T609E/E659R/D660H/W664P | |
| 605/606 | H531Q/L533S | R38G/N138R/K170L/Q246K/A311G/K336S/K340W/ | + |
| | | R357E/A388K/R394K/V401R/Y404L/L514S/H531Q/ | |
| | | L533S/T609E/E659R/D660H/W664P | |
| 607/608 | E115L | R38G/E115L/N138R/K170L/Q246K/A311G/K336S/ | + |
| | | K340W/R357E/A388K/R394K/V401R/Y404L/L514S/ | |
| | | T609E/E659R/D660H/W664P | |
| 609/610 | T83K | R38G/T83K/N138R/K170L/Q246K/A311G/K336S/ | + |
| | | K340W/R357E/A388K/R394K/V401R/Y404L/L514S/ | |
| | | T609E/E659R/D660H/W664P | |
| 611/612 | A32V/K60M/M61W | A32V/R38G/K60M/M61W/N138R/K170L/Q246K/ | + |
| | | A311G/K336S/K340W/R357E/A388K/R394K/V401R/ | |
| | | Y404L/L514S/T609E/E659R/D660H/W664P | |
| 613/614 | E55D | R38G/E55D/N138R/K170L/Q246K/A311G/K336S/ | +++ |
| | | K340W/R357E/A388K/R394K/V401R/Y404L/L514S/ | |
| | | T609E/E659R/D660H/W664P | |
| 615/616 | L589C | R38G/N138R/K170L/Q246K/A311G/K336S/K340W/ | +++ |
| | | R357E/A388K/R394K/V401R/Y404L/L514S/L589C/ | |
| | | T609E/E659R/D660H/W664P | |
| 617/618 | E357P | R38G/N138R/K170L/Q246K/A311G/K336S/K340W/ | +++ |
| | | R357P/A388K/R394K/V401R/Y404L/L514S/T609E/ | |
| | | E659R/D660H/W664P | |
| 619/620 | K617R | R38G/N138R/K170L/Q246K/A311G/K336S/K340W/ | +++ |
| | | R357E/A388K/R394K/V401R/Y404L/L514S/T609E/ | |
| | | K617R/E659R/D660H/W664P | |
| 621/622 | N172G | R38G/N138R/K170L/N172G/Q246K/A311G/K336S/ | +++ |
| | | K340W/R357E/A388K/R394K/V401R/Y404L/L514S/ | |
| | | T609E/E659R/D660H/W664P | |
| 623/624 | D598A | R38G/N138R/K170L/Q246K/A311G/K336S/K340W/ | +++ |
| | | R357E/A388K/R394K/V401R/Y404L/L514S/D598A/ | |
| | | T609E/E659R/D660H/W664P | |
| 625/626 | A56G/V193G | R38G/A56G/N138R/K170L/V193G/Q246K/A311G/ | ++ |
| | | K336S/K340W/R357E/A388K/R394K/V401R/Y404L/ | |
| | | L514S/T609E/E659R/D660H/W664P | |
| 627/628 | A56T | R38G/A56T/N138R/K170L/Q246K/A311G/K336S/ | ++ |
| | | K340W/R357E/A388K/R394K/V401R/Y404L/L514S/ | |
| | | T609E/E659R/D660H/W664P | |
| 629/630 | N536V | R38G/N138R/K170L/Q246K/A311G/K336S/K340W/ | ++ |
| | | R357E/A388K/R394K/V401R/Y404L/L514S/N536V/ | |
| | | T609E/E659R/D660H/W664P | |
| 631/632 | E52T | R38G/E52T/N138R/K170L/Q246K/A311G/K336S/ | ++ |
| | | K340W/R357E/A388K/R394K/V401R/Y404L/L514S/ | |
| | | T609E/E659R/D660H/W664P | |
| 633/634 | D17N | D17N/R38G/N138R/K170L/Q246K/A311G/K336S/ | ++ |
| | | K340W/R357E/A388K/R394K/V401R/Y404L/L514S/ | |
| | | T609E/E659R/D660H/W664P | |
| 635/636 | A56N | R38G/A56N/N138R/K170L/Q246K/A311G/K336S/ | ++ |
| | | K340W/R357E/A388K/R394K/V401R/Y404L/L514S/ | |
| | | T609E/E659R/D660H/W664P | |
| 637/638 | D598K | R38G/N138R/K170L/Q246K/A311G/K336S/K340W/ | ++ |
| | | R357E/A388K/R394K/V401R/Y404L/L514S/D598K/ | |
| | | T609E/E659R/D660H/W664P | |
| 639/640 | I261M | R38G/N138R/K170L/Q246K/I261M/A311G/K336S/ | ++ |
| | | K340W/R357E/A388K/R394K/V401R/Y404L/L514S/ | |
| | | T609E/E659R/D660H/W664P | |
| 641/642 | A56E | R38G/A56E/N138R/K170L/Q246K/A311G/K336S/ | ++ |
| | | K340W/R357E/A388K/R394K/V401R/Y404L/L514S/ | |
| | | T609E/E659R/D660H/W664P | |
| 643/644 | E55S | R38G/E55S/N138R/K170L/Q246K/A311G/K336S/ | ++ |
| | | K340W/R357E/A388K/R394K/V401R/Y404L/L514S/ | |
| | | T609E/E659R/D660H/W664P | |
| 645/646 | N536E | R38G/N138R/K170L/Q246K/A311G/K336S/K340W/ | ++ |
| | | R357E/A388K/R394K/V401R/Y404L/L514S/N536E/ | |
| | | T609E/E659R/D660H/W664P | |
| 647/648 | D598L | R38G/N138R/K170L/Q246K/A311G/K336S/K340W/ | ++ |
| | | R357E/A388K/R394K/V401R/Y404L/L514S/D598L/ | |
| | | T609E/E659R/D660H/W664P | |
| 649/650 | K78S | R38G/K78S/N138R/K170L/Q246K/A311G/K336S/ | + |
| | | K340W/R357E/A388K/R394K/V401R/Y404L/L514S/ | |
| | | T609E/E659R/D660H/W664P | |
| 651/652 | A56V | R38G/A56V/N138R/K170L/Q246K/A311G/K336S/ | + |
| | | K340W/R357E/A388K/R394K/V401R/Y404L/L514S/ | |
| | | T609E/E659R/D660H/W664P | |
| 653/654 | A629R | R38G/N138R/K170L/Q246K/A311G/K336S/K340W/ | + |
| | | R357E/A388K/R394K/V401R/Y404L/L514S/T609E/ | |
| | | A629R/E659R/D660H/W664P | |
| 655/656 | D598R | R38G/N138R/K170L/Q246K/A311G/K336S/K340W/ | + |
| | | R357E/A388K/R394K/V401R/Y404L/L514S/D598R/ | |
| | | T609E/E659R/D660H/W664P | |
| 657/658 | E63S | R38G/E63S/N138R/K170L/Q246K/A311G/K336S/ | + |
| | | K340W/R357E/A388K/R394K/V401R/Y404L/L514S/ | |
| | | T609E/E659R/D660H/W664P | |
| 659/660 | T82S | R38G/T82S/N138R/K170L/Q246K/A311G/K336S/ | + |
| | | K340W/R357E/A388K/R394K/V401R/Y404L/L514S/ | |
| | | T609E/E659R/D660H/W664P | |
| 661/662 | A207S | R38G/N138R/K170L/A207S/Q246K/A311G/K336S/ | + |
| | | K340W/R357E/A388K/R394K/V401R/Y404L/L514S/ | |
| | | T609E/E659R/D660H/W664P | |
| 663/664 | D598V | R38G/N138R/K170L/Q246K/A311G/K336S/K340W/ | + |
| | | R357E/A388K/R394K/V401R/Y404L/L514S/D598V/ | |
| | | T609E/E659R/D660H/W664P | |
| 665/666 | I666M | R38G/N138R/K170L/Q246K/A311G/K336S/K340W/ | + |
| | | R357E/A388K/R394K/V401R/Y404L/L514S/T609E/ | |
| | | E659R/D660H/W664P/I666M | |
| 667/668 | R150Y | R38G/N138R/R150Y/K170L/Q246K/A311G/K336S/ | + |
| | | K340W/R357E/A388K/R394K/V401R/Y404L/L514S/ | |
| | | T609E/E659R/D660H/W664P | |
| 669/670 | A45K | R38G/A45K/N138R/K170L/Q246K/A311G/K336S/ | + |
| | | K340W/R357E/A388K/R394K/V401R/Y404L/L514S/ | |
| | | T609E/E659R/D660H/W664P | |
| 671/672 | E55W | R38G/E55W/N138R/K170L/Q246K/A311G/K336S/ | + |
| | | K340W/R357E/A388K/R394K/V401R/Y404L/L514S/ | |
| | | T609E/E659R/D660H/W664P | |
| 673/674 | S135G | R38G/S135G/N138R/K170L/Q246K/A311G/K336S/ | + |
| | | K340W/R357E/A388K/R394K/V401R/Y404L/L514S/ | |
| | | T609E/E659R/D660H/W664P | |
| 675/676 | I13N | I13N/R38G/N138R/K170L/Q246K/A311G/K336S/ | + |
| | | K340W/R357E/A388K/R394K/V401R/Y404L/L514S/ | |
| | | T609E/E659R/D660H/W664P | |
| 677/678 | D33E | D33E/R38G/N138R/K170L/Q246K/A311G/K336S/ | + |
| | | K340W/R357E/A388K/R394K/V401R/Y404L/L514S/ | |
| | | T609E/E659R/D660H/W664P | |
| 679/680 | G245C | R38G/N138R/K170L/G245C/Q246K/A311G/K336S/ | + |
| | | K340W/R357E/A388K/R394K/V401R/Y404L/L514S/ | |
| | | T609E/E659R/D660H/W664P | |

Levels of increased activity were determined relative to the reference polypeptide of SEQ ID NO: 588 and defined as follows: “+” 1.01 to 1.35 (first 50%), “++” >1.35 (next 30%), “+++” >1.83 (top 20%)

### Example 17

### Improvements Over RNA Polymerase of SEQ ID NO: 604

SEQ ID NO: 604 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 11 kb-DNA template, 40 mM Tris-HCl pH 8.0, 50 mM magnesium acetate, 12 mM ATP, 12 mM GTP, 12 mM CTP, 12 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 604 (Activity FIOP) was calculated as the RNA produced by variant over the RNA produced by SEQ ID NO: 604. The result is shown in Table 17.1.

TABLE 17.1
Activities of Variants Relative to SEQ ID NO: 604

| | Amino Acid | Amino Acid | |
|---|---|---|---|
| SEQ ID | Differences | Differences | FIOP Product |
| NO: | (Relative to | (Relative to | Relative to SEQ |
| (nt/aa) | SEQ ID NO: 604) | SEQ ID NO: 2) | ID NO: 604 |
| 681/682 | A207S/G245C/A629R | A32V/R38G/N138R/K170L/A207S/G245C/ | +++ |
| | | Q246K/A311G/K336S/K340W/R357E/A388K/ | |
| | | R394K/V401R/Y404L/L514S/T609E/A629R/ | |
| | | E659R/D660H/W664P | |
| 683/684 | D17N/A56E/R150Y/ | D17N/A32V/R38G/A56E/N138R/R150Y/K170L/ | ++ |
| | D598A | Q246K/A311G/K336S/K340W/R357E/A388K/ | |
| | | R394K/V401R/Y404L/L514S/D598A/T609E/ | |
| | | E659R/D660H/W664P | |
| 685/686 | A56E/A629R | A32V/R38G/A56E/N138R/K170L/Q246K/ | ++ |
| | | A311G/K336S/K340W/R357E/A388K/R394K/ | |
| | | V401R/Y404L/L514S/T609E/A629R/E659R/ | |
| | | D660H/W664P | |
| 687/688 | N536V | A32V/R38G/N138R/K170L/Q246K/A311G/ | ++ |
| | | K336S/K340W/R357E/A388K/R394K/V401R/ | |
| | | Y404L/L514S/N536V/T609E/E659R/D660H/ | |
| | | W664P | |
| 689/690 | Y185P | A32V/R38G/N138R/K170L/Y185P/Q246K/ | ++ |
| | | A311G/K336S/K340W/R357E/A388K/R394K/ | |
| | | V401R/Y404L/L514S/T609E/E659R/D660H/ | |
| | | W664P | |
| 691/692 | A56T/R150Y | A32V/R38G/A56T/N138R/R150Y/K170L/ | + |
| | | Q246K/A311G/K336S/K340W/R357E/A388K/ | |
| | | R394K/V401R/Y404L/L514S/T609E/E659R/ | |
| | | D660H/W664P | |
| 693/694 | D17N/A56E/R150Y/ | D17N/A32V/R38G/A56E/N138R/R150Y/K170L/ | + |
| | A207S/G245C/D598A/ | A207S/G245C/Q246K/A311G/K336S/K340W/ | |
| | K617R/A629R | R357E/A388K/R394K/V401R/Y404L/L514S/ | |
| | | D598A/T609E/K617R/A629R/E659R/D660H/ | |
| | | W664P | |
| 695/696 | A56E/R150Y/N536V/ | A32V/R38G/A56E/N138R/R150Y/K170L/ | + |
| | D598K | Q246K/A311G/K336S/K340W/R357E/A388K/ | |
| | | R394K/V401R/Y404L/L514S/N536V/D598K/ | |
| | | T609E/E659R/D660H/W664P | |
| 697/698 | A56V/N536V | A32V/R38G/A56V/N138R/K170L/Q246K/ | + |
| | | A311G/K336S/K340W/R357E/A388K/R394K/ | |
| | | V401R/Y404L/L514S/N536V/T609E/E659R/ | |
| | | D660H/W664P | |
| 699/700 | Y185P/I666M | A32V/R38G/N138R/K170L/Y185P/Q246K/ | + |
| | | A311G/K336S/K340W/R357E/A388K/R394K/ | |
| | | V401R/Y404L/L514S/T609E/E659R/D660H/ | |
| | | W664P/I666M | |
| 701/702 | R150Y | A32V/R38G/N138R/R150Y/K170L/Q246K/ | +++ |
| | | A311G/K336S/K340W/R357E/A388K/R394K/ | |
| | | V401R/Y404L/L514S/T609E/E659R/D660H/ | |
| | | W664P | |
| 703/704 | A143N/R150Y/K246V/ | A32V/R38G/N138R/A143N/R150Y/K170L/ | + |
| | E782V | Q246V/A311G/K336S/K340W/R357E/A388K/ | |
| | | R394K/V401R/Y404L/L514S/T609E/E659R/ | |
| | | D660H/W664P/E782V | |

Levels of increased activity were determined relative to the reference polypeptide of SEQ ID NO: 604 and
defined as follows: “+” 1.01 to 1.06 (first 50%), “++” > 1.06 (next 30%), “+++” > 1.17 (top 20%)

### Example 18

### Improvements Over RNA Polymerase of SEQ ID NO: 686

SEQ ID NO: 686 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 11 kb-DNA template, 40 mM Tris-HCl pH 8.0, 50 mM magnesium acetate, 12 mM ATP, 12 mM GTP, 12 mM CTP, 12 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 686 (Activity FIOP) was calculated as the RNA produced by variant over the RNA produced by SEQ ID NO: 686. The result is shown in Table 18.1.

TABLE 18.1
Activities of Variants Relative to SEQ ID NO: 686

| | Amino Acid | Amino Acid | |
|---|---|---|---|
| SEQ ID | Differences | Differences | FIOP Product |
| NO: | (Relative to | (Relative to | Relative to SEQ |
| (nt/aa) | SEQ ID NO: 686) | SEQ ID NO: 2) | ID NO: 686 |
| 705/706 | A143N | A32V/R38G/A56E/N138R/A143N/K170L/Q246K/ | +++ |
| | | A311G/K336S/K340W/R357E/A388K/R394K/ | |
| | | V401R/Y404L/L514S/T609E/A629R/E659R/ | |
| | | D660H/W664P | |
| 707/708 | K246R/P424A/ | A32V/R38G/A56E/N138R/K170L/Q246R/A311G/ | +++ |
| | S648F/E782V | K336S/K340W/R357E/A388K/R394K/V401R/ | |
| | | Y404L/P424A/L514S/T609E/A629R/S648F/E659R/ | |
| | | D660H/W664P/E782V | |
| 709/710 | K246R/R659E/ | A32V/R38G/A56E/N138R/K170L/Q246R/A311G/ | ++ |
| | E782V | K336S/K340W/R357E/A388K/R394K/V401R/Y404L/ | |
| | | L514S/T609E/A629R/D660H/W664P/E782V | |
| 711/712 | E56T/R150Y/R629A/ | A32V/R38G/A56T/N138R/R150Y/K170L/Q246K/ | ++ |
| | H660W/E782V | A311G/K336S/K340W/R357E/A388K/R394K/V401R/ | |
| | | Y404L/L514S/T609E/E659R/D660W/W664P/E782V | |
| 713/714 | K721T/E782V | A32V/R38G/A56E/N138R/K170L/Q246K/A311G/ | + |
| | | K336S/K340W/R357E/A388K/R394K/V401R/Y404L/ | |
| | | L514S/T609E/A629R/E659R/D660H/W664P/K721T/ | |
| | | E782V | |
| 715/716 | E782V | A32V/R38G/A56E/N138R/K170L/Q246K/A311G/ | + |
| | | K336S/K340W/R357E/A388K/R394K/V401R/Y404L/ | |
| | | L514S/T609E/A629R/E659R/D660H/W664P/E782V | |
| 717/718 | D196G/R659E/E782V | A32V/R38G/A56E/N138R/K170L/D196G/Q246K/ | + |
| | | A311G/K336S/K340W/R357E/A388K/R394K/V401R/ | |
| | | Y404L/L514S/T609E/A629R/D660H/W664P/E782V | |
| 719/720 | K720S/K721A/E782V | A32V/R38G/A56E/N138R/K170L/Q246K/A311G/ | + |
| | | K336S/K340W/R357E/A388K/R394K/V401R/Y404L/ | |
| | | L514S/T609E/A629R/E659R/D660H/W664P/K720S/ | |
| | | K721A/E782V | |

Levels of increased activity were determined relative to the reference polypeptide of SEQ ID NO: 686 and defined as follows: “+” 1.13 to 1.25 (first 50%), “++” >1.25 (next 30%), “+++” >1.40 (top 20%)

### Example 19

### Improvements Over RNA Polymerase of SEQ ID NO: 692

SEQ ID NO: 692 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 11 kb-DNA template, 40 mM Tris-HCl pH 8.0, 50 mM magnesium acetate, 12 mM ATP, 12 mM GTP, 12 mM CTP, 12 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Catalog #Q10213). Activity relative to SEQ ID NO: 692 (Activity FIOP) was calculated as the RNA produced by variant over the RNA produced by SEQ ID NO: 692. The result is shown in Table 19.1.

TABLE 18.1
Activities of Variants Relative to SEQ ID NO: 692

| | Amino Acid | Amino Acid | |
|---|---|---|---|
| SEQ ID | Differences | Differences | FIOP Product |
| NO: | (Relative to | (Relative to | Relative to SEQ |
| (nt/aa) | SEQ ID NO: 692) | SEQ ID NO: 2) | ID NO: 692 |
| 721/722 | A143N/R659E/ | A32V/R38G/A56T/N138R/A143N/R150Y/K170L/ | + |
| | E782V | Q246K/A311G/K336S/K340W/R357E/A388K/R394K/ | |
| | | V401R/Y404L/L514S/T609E/D660H/W664P/E782V | |
| 723/724 | A166T/K171E/ | A32V/R38G/A56T/N138R/R150Y/A166T/K170L/ | + |
| | K246R/G311A | K171E/Q246R/K336S/K340W/R357E/A388K/R394K/ | |
| | | V401R/Y404L/L514S/T609E/E659R/D660H/W664P | |
| 725/726 | A143N/K721T | A32V/R38G/A56T/N138R/A143N/R150Y/K170L/ | + |
| | | Q246K/A311G/K336S/K340W/R357E/A388K/R394K/ | |
| | | V401R/Y404L/L514S/T609E/E659R/D660H/W664P/ | |
| | | K721T | |
| 727/728 | E782V | A32V/R38G/A56T/N138R/R150Y/K170L/Q246K/ | + |
| | | A311G/K336S/K340W/R357E/A388K/R394K/V401R/ | |
| | | Y404L/L514S/T609E/E659R/D660H/W664P/E782V | |
| 729/730 | A143N/K171E/ | A32V/R38G/A56T/N138R/A143N/R150Y/K170L/ | + |
| | R659E/E782V | K171E/Q246K/A311G/K336S/K340W/R357E/A388K/ | |
| | | R394K/V401R/Y404L/L514S/T609E/D660H/W664P/ | |
| | | E782V | |

Levels of increased activity were determined relative to the reference polypeptide of SEQ ID NO: 692 and defined as follows: “+” from 1.24 to 1.42

### Example 20

### Improvements Over RNA Polymerase of SEQ ID NO: 2

The polypeptides encoded by the following genes were generated and purified from the shake flask cultures as described in Example 4. Each variant was assayed in a 200 μL reaction that comprised 0.05 μg/μL 9 kb-DNA template, 30 mM Tris-HCl pH 8.0, 26.9 mM magnesium chloride, 5 mM ATP, 5 mM GTP, 5 mM CTP, 5 mM UTP, 1 U/μL Ribolock RNase Inhibitor (Thermo Scientific, Catalog #E00382), 0.002 U/μL Pyrophosphatase (Thermo Scientific, Catalog #EF0221), and a final concentration of 20 ng/μL of variants. The IVI reactions were initiated by adding the variants, incubated in Bio-Rad Hard-Shell® 96-Well PCR plates at 25° C. for 2 h, and quenched with 25 μL of 0.5 MEDA. Quenched mixtures were diluted 40-fold with TE buffer (10 mM Tris-HCl pH 7.0 and 0.1 mM EDTA) and analyzed on the Fragment Analyzer (Agilent 5300 Fragment Analyzer) with the RNA Kit (Agilent DNF-471-1000). Integrity relative to SEQ ID NO: 2 (Integrity FIOP) was calculated as the integrity of RNA produced by variant over the integrity of RNA produced by SEQ ID NO: 2. The result is shown in Table 20.1.

TABLE 20.1
Integrities of Variants Relative to SEQ ID NO: 2

| | Amino Acid | |
|---|---|---|
| SEQ ID | Differences | FIOP Product |
| NO: | (Relative to | Relative to SEQ ID |
| (nt/aa) | SEQ ID NO: 2) | NO: 2 |
| 31/32 | N138R/A311G/K336S/K340W/A388K/V401R/Y404L | + |
| 41/42 | N138R/A311G/K336S/K340W/A388K/V401R/Y404L/L514S | + |
| 43/44 | R38G/N138R/A311G/K336S/K340W/A388K/V401R/Y404L/L514S | + |
| 153/154 | N138R/K170Q/A311G/K336S/A388K/V401R/Y404L/L514S/E659R/ | + |
| | D660H/G810K | |
| 149/150 | N138R/V173Y/A311G/K336S/K340W/A388K/V401R/Y404L/L514S/ | + |
| | E659R/D660H | |

Levels of increased activity were determined relative to the reference polypeptide of SEQ ID NO: 2 and defined as follows: “+” from 2.72 to 3.33

### Example 21

### Improvements Over RNA Polymerase of SEQ ID NO: 150

The polypeptides encoded by the following genes were generated and purified from the shake flask cultures as described in Example 4. Each variant was assayed in a 200 μL reaction that comprised 0.05 μg/μL 8 kb-DNA template, 40 mM Tris-HCl pH 8.0, 30 mM magnesium acetate, 7.5 mM ATP, 7.5 mM GTP, 7.5 mM CTP, 7.5 mM UTP, 1 U/μL Ribolock RNase Inhibitor (Thermo Scientific, Catalog #E00382), 0.002 U/μL Pyrophosphatase (Thermo Scientific, Catalog #EF0221), and a final concentration of 20 ng/μL of variants. The IVT reactions were initiated by adding the variants, incubated in Bio-Rad Hard-Shell® 96-Well PCR plates at 25° C. for 2 h, and quenched with 25 μL of 0.5 M EDTA. Quenched mixtures were diluted 40-fold with TE buffer (10 mM Tris-HCl pH 7.0 and 0.1 mM EDTA) and analyzed on the Fragment Analyzer (Agilent 5300 Fragment Analyzer) with the RNA Kit (Agilent DNF-471-1000). Integrity relative to SEQ ID NO: 150 (Integrity FIOP) was calculated as the integrity of RNA produced by variant over the integrity of RNA produced by SEQ ID NO: 150. The result is shown in Table 21.1.

TABLE 21.1
Integrities of Variants Relative to SEQ ID NO: 150

| | | Amino Acid | FIOP Product |
|---|---|---|---|
| SEQ ID | Amino Acid Differences | Differences | Relative to |
| NO: | (Relative to | (Relative to | SEQ ID NO: |
| (nt/aa) | SEQ ID NO: 150) | SEQ ID NO: 2) | 150 |
| 393/394 | R38G/K170L/Y173V/ | R38G/N138R/K170L/A311G/K336S/K340W/ | + |
| | R357E/R394K/W664P | R357E/A388K/R394K/V401R/Y404L/L514S/E659R/ | |
| | | D660H/W664P | |
| 587/588 | R38G/K170L/Y173V/ | R38G/N138R/K170L/Q246K/A311G/K336S/K340W/ | + |
| | Q246K/R357E/R394K/ | R357E/A388K/R394K/V401R/Y404L/L514S/ | |
| | T609E/W664P | T609E/E659R/D660H/W664P | |
| 603/604 | A32V/R38G/K170L/ | A32V/R38G/N138R/K170L/Q246K/A311G/K336S/ | + |
| | Y173V/Q246K/R357E/ | K340W/R357E/A388K/R394K/V401R/Y404L/ | |
| | R394K/T609E/W664P | L514S/T609E/E659R/D660H/W664P | |

Levels of increased activity were determined relative to the reference polypeptide of SEQ ID NO: 150 and defined as follows: “+” from 1.11 to 1.43

### Example 22

### Improvements Over SEQ ID NO: 150 in RNA Polymerase Integrity

The polypeptides encoded by the following genes were generated and purified from the shake flask cultures as described in Example 4. Each variant was assayed in a 200 μL reaction that comprised 0.05 μg/μL 11 kb-DNA template, 40 mM Tris-HCl pH 8.0, 40 mM magnesium acetate, 10 mM ATP, 10 mM GTP, 10 mM CTP, 10 mM UTP, 1 U/μL Ribolock RNase Inhibitor (Thermo Scientific, Catalog #E00382), 0.002 U/μL Pyrophosphatase (Thermo Scientific, Catalog #EF0221), and a final concentration of 20 ng/μL of variants. The IVT reactions were initiated by adding the variants, incubated in Bio-Rad Hard-Shell® 96-Well PCR plates at 25° C. for 2 h, and quenched with 25 μL of 0.5 M EDTA. Quenched mixtures were diluted 40-fold with TE buffer (10 mM Tris-HCl pH 7.0 and 0.1 mM EDTA) and analyzed on the Fragment Analyzer (Agilent 5300 Fragment Analyzer) with the RNA Kit (Agilent DNF-471-1000). Integrity relative to SEQ ID NO: 150 (Integrity FIOP) was calculated as the integrity of RNA produced by variant over the integrity of RNA produced by SEQ ID NO: 150. The result is shown in Table 22.1.

TABLE 22.1
Integrities of Variants Relative to SEQ ID NO: 150

| | Amino Acid | Amino Acid | FIOP Product |
|---|---|---|---|
| SEQ ID | Differences | Differences | Relative to |
| NO: | (Relative to | (Relative to | SEQ ID NO: |
| (nt/aa) | SEQ ID NO: 150) | SEQ ID NO: 2) | 150 |
| 393/394 | R38G/K170L/Y173V/ | R38G/N138R/K170L/A311G/K336S/K340W/ | + |
| | R357E/R394K/W664P | R357E/A388K/R394K/V401R/Y404L/L514S/E659R/ | |
| | | D660H/W664P | |
| 587/588 | R38G/K170L/Y173V/ | R38G/N138R/K170L/Q246K/A311G/K336S/K340W/ | + |
| | Q246K/R357E/R394K/ | R357E/A388K/R394K/V401R/Y404L/L514S/T609E/ | |
| | T609E/W664P | E659R/D660H/W664P | |
| 603/604 | A32V/R38G/K170L/ | A32V/R38G/N138R/K170L/Q246K/A311G/K336S/ | ++ |
| | Y173V/Q246K/R357E/ | K340W/R357E/A388K/R394K/V401R/Y404L/L514S/ | |
| | R394K/T609E/W664P | T609E/E659R/D660H/W664P | |
| 685/686 | A32V/R38G/A56E/K170L/ | A32V/R38G/A56E/N138R/K170L/Q246K/A311G/ | ++ |
| | Y173V/Q246K/R357E/ | K336S/K340W/R357E/A388K/R394K/V401R/Y404L/ | |
| | R394K/T609E/A629R/ | L514S/T609E/A629R/E659R/D660H/W664P | |
| | W664P | | |
| 705/706 | A32V/R38G/A56E/A143N/ | A32V/R38G/A56E/N138R/A143N/K170L/Q246K/ | +++ |
| | K170L/Y173V/Q246K/ | A311G/K336S/K340W/R357E/A388K/R394K/ | |
| | R357E/R394K/T609E/ | V401R/Y404L/L514S/T609E/A629R/E659R/D660H/ | |
| | A629R/W664P | W664P | |
| 727/728 | A32V/R38G/A56T/R150Y/ | A32V/R38G/A56T/N138R/R150Y/K170L/Q246K/ | + |
| | K170L/Y173V/Q246K/ | A311G/K336S/K340W/R357E/A388K/R394K/ | |
| | R357E/R394K/T609E/ | V401R/Y404L/L514S/T609E/E659R/D660H/W664P/ | |
| | W664P/E782V | E782V | |

Levels of increased activity were determined relative to the reference polypeptide of SEQ ID NO: 2 and defined as follows: “+” .98 to 1.12 (first 50%), “++” >1.12 (next 30%), “+++” >2.24 (top 20%)

### Example 23

### Improvements Over SEQ ID NO: 712 in RNA Polymerase Integrity

SEQ ID NO: 712 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HTP as described in Example 2 and purified as described in Example 3. Each variant was diluted 4-fold in RNase-free water and screened in a 1 μL reaction that comprised 0.05 μg/μL 11 kb-DNA template, 40 mM Tris-HCl pH 8.0, 50 mM magnesium acetate, 12 mM ATP, 12 mM GTP, 12 mM CTP, 12 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 712 (Activity FIOP) was calculated as the RNA produced by variant over the RNA produced by SEQ ID NO: 712. The result is shown in Table 23.1.

TABLE 23.1
Activities of Variants Relative to SEQ ID NO: 712

| | Amino Acid | Amino Acid | |
|---|---|---|---|
| SEQ ID | Differences | Differences | FIOP Product |
| NO: | (Relative to SEQ | (Relative to | Relative to SEQ |
| (nt/aa) | ID NO: 712) | SEQ ID NO: 2) | ID NO: 712 |
| 731/732 | L367P | A32V/R38G/A56T/N138R/R150Y/K170L/Q246K/A311G/ | +++ |
| | | K336S/K340W/R357E/L367P/A388K/R394K/V401R/ | |
| | | Y404L/L514S/T609E/E659R/D660W/W664P/E782V | |
| 733/734 | T382R | A32V/R38G/A56T/N138R/R150Y/K170L/Q246K/A311G/ | +++ |
| | | K336S/K340W/R357E/T382R/A388K/R394K/V401R/ | |
| | | Y404L/L514S/T609E/E659R/D660W/W664P/E782V | |
| 735/736 | K670R | A32V/R38G/A56T/N138R/R150Y/K170L/Q246K/A311G/ | +++ |
| | | K336S/K340W/R357E/A388K/R394K/V401R/Y404L/ | |
| | | L514S/T609E/E659R/D660W/W664P/K670R/E782V | |
| 737/738 | T382L | A32V/R38G/A56T/N138R/R150Y/K170L/Q246K/A311G/ | +++ |
| | | K336S/K340W/R357E/T382L/A388K/R394K/V401R/ | |
| | | Y404L/L514S/T609E/E659R/D660W/W664P/E782V | |
| 739/740 | L672P | A32V/R38G/A56T/N138R/R150Y/K170L/Q246K/A311G/ | +++ |
| | | K336S/K340W/R357E/A388K/R394K/V401R/Y404L/ | |
| | | L514S/T609E/E659R/D660W/W664P/L672P/E782V | |
| 741/742 | E357P | A32V/R38G/A56T/N138R/R150Y/K170L/Q246K/A311G/ | +++ |
| | | K336S/K340W/R357P/A388K/R394K/V401R/Y404L/ | |
| | | L514S/T609E/E659R/D660W/W664P/E782V | |
| 743/744 | K388R | A32V/R38G/A56T/N138R/R150Y/K170L/Q246K/A311G/ | +++ |
| | | K336S/K340W/R357E/A388R/R394K/V401R/Y404L/ | |
| | | L514S/T609E/E659R/D660W/W664P/E782V | |
| 745/746 | S693R | A32V/R38G/A56T/N138R/R150Y/K170L/Q246K/A311G/ | ++ |
| | | K336S/K340W/R357E/A388K/R394K/V401R/Y404L/ | |
| | | L514S/T609E/E659R/D660W/W664P/S693R/E782V | |
| 747/748 | L672S | A32V/R38G/A56T/N138R/R150Y/K170L/Q246K/A311G/ | ++ |
| | | K336S/K340W/R357E/A388K/R394K/V401R/Y404L/ | |
| | | L514S/T609E/E659R/D660W/W664P/L672S/E782V | |
| 749/750 | A397S | A32V/R38G/A56T/N138R/R150Y/K170L/Q246K/A311G/ | ++ |
| | | K336S/K340W/R357E/A388K/R394K/A397S/V401R/ | |
| | | Y404L/L514S/T609E/E659R/D660W/W664P/E782V | |
| 751/752 | D375P | A32V/R38G/A56T/N138R/R150Y/K170L/Q246K/A311G/ | ++ |
| | | K336S/K340W/R357E/D375P/A388K/R394K/V401R/ | |
| | | Y404L/L514S/T609E/E659R/D660W/W664P/E782V | |
| 753/754 | L672G | A32V/R38G/A56T/N138R/R150Y/K170L/Q246K/A311G/ | ++ |
| | | K336S/K340W/R357E/A388K/R394K/V401R/Y404L/ | |
| | | L514S/T609E/E659R/D660W/W664P/L672G/E782V | |
| 755/756 | D137G/T382R/ | A32V/R38G/A56T/D137G/N138R/R150Y/K170L/Q246K/ | ++ |
| | V782E | A311G/K336S/K340W/R357E/T382R/A388K/R394K/ | |
| | | V401R/Y404L/L514S/T609E/E659R/D660W/W664P | |
| 757/758 | Q655R | A32V/R38G/A56T/N138R/R150Y/K170L/Q246K/A311G/ | ++ |
| | | K336S/K340W/R357E/A388K/R394K/V401R/Y404L/ | |
| | | L514S/T609E/Q655R/E659R/D660W/W664P/E782V | |
| 759/760 | D137G/T382R/ | A32V/R38G/A56T/D137G/N138R/R150Y/K170L/Q246K/ | ++ |
| | Y392A | A311G/K336S/K340W/R357E/T382R/A388K/Y392A/ | |
| | | R394K/V401R/Y404L/L514S/T609E/E659R/D660W/ | |
| | | W664P/E782V | |
| 761/762 | V391F | A32V/R38G/A56T/N138R/R150Y/K170L/Q246K/A311G/ | ++ |
| | | K336S/K340W/R357E/A388K/V391F/R394K/V401R/ | |
| | | Y404L/L514S/T609E/E659R/D660W/W664P/E782V | |
| 763/764 | D137G/Q655M/ | A32V/R38G/A56T/D137G/N138R/R150Y/K170L/Q246K/ | + |
| | K721T/R763S | A311G/K336S/K340W/R357E/A388K/R394K/V401R/ | |
| | | Y404L/L514S/T609E/Q655M/E659R/D660W/W664P/ | |
| | | K721T/R763S/E782V | |
| 765/766 | V696L | A32V/R38G/A56T/N138R/R150Y/K170L/Q246K/A311G/ | + |
| | | K336S/K340W/R357E/A388K/R394K/V401R/Y404L/ | |
| | | L514S/T609E/E659R/D660W/W664P/V696L/E782V | |
| 767/768 | L672R | A32V/R38G/A56T/N138R/R150Y/K170L/Q246K/A311G/ | + |
| | | K336S/K340W/R357E/A388K/R394K/V401R/Y404L/ | |
| | | L514S/T609E/E659R/D660W/W664P/L672R/E782V | |
| 769/770 | Q663L | A32V/R38G/A56T/N138R/R150Y/K170L/Q246K/A311G/ | + |
| | | K336S/K340W/R357E/A388K/R394K/V401R/Y404L/ | |
| | | L514S/T609E/E659R/D660W/Q663L/W664P/E782V | |
| 771/772 | L367T | A32V/R38G/A56T/N138R/R150Y/K170L/Q246K/A311G/ | + |
| | | K336S/K340W/R357E/L367T/A388K/R394K/V401R/ | |
| | | Y404L/L514S/T609E/E659R/D660W/W664P/E782V | |
| 773/774 | L367F | A32V/R38G/A56T/N138R/R150Y/K170L/Q246K/A311G/ | + |
| | | K336S/K340W/R357E/L367F/A388K/R394K/V401R/ | |
| | | Y404L/L514S/T609E/E659R/D660W/W664P/E782V | |
| 775/776 | D137G/T382G | A32V/R38G/A56T/D137G/N138R/R150Y/K170L/Q246K/ | + |
| | | A311G/K336S/K340W/R357E/T382G/A388K/R394K/ | |
| | | V401R/Y404L/L514S/T609E/E659R/D660W/W664P/ | |
| | | E782V | |
| 777/778 | D137G/T382G/ | A32V/R38G/A56T/D137G/N138R/R150Y/K170L/Q246K/ | + |
| | Y392R/E650M/ | A311G/K336S/K340W/R357E/T382G/A388K/Y392R/ | |
| | Q655M/V782E | R394K/V401R/Y404L/L514S/T609E/E650M/Q655M/ | |
| | | E659R/D660W/W664P | |
| 779/780 | D137G/T382R/ | A32V/R38G/A56T/D137G/N138R/R150Y/K170L/Q246K/ | + |
| | A629R/W660Y/ | A311G/K336S/K340W/R357E/T382R/A388K/R394K/ | |
| | L672T | V401R/Y404L/L514S/T609E/A629R/E659R/D660Y/ | |
| | | W664P/L672T/E782V | |
| 781/782 | V391S | A32V/R38G/A56T/N138R/R150Y/K170L/Q246K/ | + |
| | | A311G/K336S/K340W/R357E/A388K/V391S/R394K/ | |
| | | V401R/Y404L/L514S/T609E/E659R/D660W/W664P/ | |
| | | E782V | |
| 783/784 | V616A | A32V/R38G/A56T/N138R/R150Y/K170L/Q246K/A311G/ | + |
| | | K336S/K340W/R357E/A388K/R394K/V401R/Y404L/ | |
| | | L514S/T609E/V616A/E659R/D660W/W664P/E782V | |
| 785/786 | E690S | A32V/R38G/A56T/N138R/R150Y/K170L/Q246K/A311G/ | + |
| | | K336S/K340W/R357E/A388K/R394K/V401R/Y404L/ | |
| | | L514S/T609E/E659R/D660W/W664P/E690S/E782V | |
| 787/788 | K179N | A32V/R38G/A56T/N138R/R150Y/K170L/K179N/Q246K/ | + |
| | | A311G/K336S/K340W/R357E/A388K/R394K/V401R/ | |
| | | Y404L/L514S/T609E/E659R/D660W/W664P/E782V | |
| 789/790 | D137G | A32V/R38G/A56T/D137G/N138R/R150Y/K170L/Q246K/ | + |
| | | A311G/K336S/K340W/R357E/A388K/R394K/V401R/ | |
| | | Y404L/L514S/T609E/E659R/D660W/W664P/E782V | |
| 791/792 | E357P/T382R/ | A32V/R38G/A56T/N138R/R150Y/K170L/Q246K/A311G/ | ++ |
| | K670R | K336S/K340W/R357P/T382R/A388K/R394K/V401R/ | |
| | | Y404L/L514S/T609E/E659R/D660W/W664P/K670R/ | |
| | | E782V | |
| 793/794 | K388R/K670R/ | A32V/R38G/A56T/N138R/R150Y/K170L/Q246K/A311G/ | ++ |
| | L672P | K336S/K340W/R357E/A388R/R394K/V401R/Y404L/ | |
| | | L514S/T609E/E659R/D660W/W664P/K670R/L672P/ | |
| | | E782V | |
| 795/796 | E357P/D375P | A32V/R38G/A56T/N138R/R150Y/K170L/Q246K/A311G/ | + |
| | | K336S/K340W/R357P/D375P/A388K/R394K/V401R/ | |
| | | Y404L/L514S/T609E/E659R/D660W/W664P/E782V | |
| 797/798 | E357P/L672G | A32V/R38G/A56T/N138R/R150Y/K170L/Q246K/A311G/ | + |
| | | K336S/K340W/R357P/A388K/R394K/V401R/Y404L/ | |
| | | L514S/T609E/E659R/D660W/W664P/L672G/E782V | |
| 799/800 | A629K | A32V/R38G/A56T/N138R/R150Y/K170L/Q246K/A311G/ | + |
| | | K336S/K340W/R357E/A388K/R394K/V401R/Y404L/ | |
| | | L514S/T609E/A629K/E659R/D660W/W664P/E782V | |
| 801/802 | Q655R/K670R | A32V/R38G/A56T/N138R/R150Y/K170L/Q246K/A311G/ | + |
| | | K336S/K340W/R357E/A388K/R394K/V401R/Y404L/ | |
| | | L514S/T609E/Q655R/E659R/D660W/W664P/K670R/ | |
| | | E782V | |

Levels of increased activity were determined relative to the reference polypeptide of SEQ ID NO: 712 and defined as follows: “+” 1.01 to 1.17 (first 50%), “++” >1.17 (next 30%), “+++” >1.42 (top 20%)

### Example 24

### Improvements Over SEQ ID NO: 800 in RNA Polymerase Processivity

SEQ ID NO: 800 was selected as the parent enzyme in the in vitro transcription (IVT) assay. Libraries of engineered genes were produced using known techniques (e.g., saturation mutagenesis, recombination of previously identified beneficial mutations). The polypeptides encoded by each gene were produced in HIP as described in Example 2 and purified as described in Example 3. Each variant was diluted 4-fold in RNase-free water and screened in a 1 μL reaction that comprised 0.05 μg/μL 11 kb-DNA template, 40 mM Tris-HCl pH 8.0, 50 mM magnesium acetate, 12 mM ATP, 12 mM GTP, 12 mM CTP, 12 mM UTP, 1 U/μL Ribolock RNase Inhibitor (Thermo Scientific, Catalog #E00382), and 0.002 U/μL Pyrophosphatase (Thermo Scientific, Catalog #EF0221). The reactions were initiated by adding 150 nL of the HIP purified variants, incubated in Bio-Rad Hard-Shell® 96-Well PCR plates at 25° C. for 2 h and quenched with 125 nL of 0.5 M EDTA. Quenched mixtures were diluted 100-fold with RNase-free water, and RNA concentrations were measure by using the Quant-iT™ RNA Assay Kits—Broad Range (Thermo Fisher, Catalog #Q10213). Activity relative to SEQ ID NO: 800 (Activity FIOP) was calculated as the RNA produced by variant over the RNA produced by SEQ ID NO: 800. The result is shown in Table 24.1.

TABLE 24.1
Activities of Variants Relative to SEQ ID NO: 800

| | Amino Acid | Amino Acid | |
|---|---|---|---|
| SEQ ID | Differences | Differences | FIOP Product |
| NO: | (Relative to SEQ | (Relative to | Relative to SEQ |
| (nt/aa) | ID NO: 800) | SEQ ID NO: 2) | ID NO: 800 |
| 803/804 | D137G | A32V/R38G/A56T/D137G/N138R/R150Y/K170L/Q246K/ | +++ |
| | | A311G/K336S/K340W/R357E/A388K/R394K/V401R/ | |
| | | Y404L/L514S/T609E/A629K/E659R/D660W/W664P/ | |
| | | E782V | |
| 805/806 | T56E/G311A/ | A32V/R38G/A56E/N138R/R150Y/K170L/Q246K/K336S/ | +++ |
| | T382R | K340W/R357E/T382R/A388K/R394K/V401R/Y404L/ | |
| | | L514S/T609E/A629K/E659R/D660W/W664P/E782V | |
| 807/808 | T56E/D137G/ | A32V/R38G/A56E/D137G/N138R/A143N/R150Y/K170L/ | ++ |
| | A143N/K721A | Q246K/A311G/K336S/K340W/R357E/A388K/R394K/ | |
| | | V401R/Y404L/L514S/T609E/A629K/E659R/D660W/ | |
| | | W664P/K721A/E782V | |
| 809/810 | D137G/Y392A | A32V/R38G/A56T/D137G/N138R/R150Y/K170L/Q246K/ | ++ |
| | | A311G/K336S/K340W/R357E/A388K/Y392A/R394K/ | |
| | | V401R/Y404L/L514S/T609E/A629K/E659R/D660W/ | |
| | | W664P/E782V | |
| 811/812 | T56E/D137G/ | A32V/R38G/A56E/D137G/N138R/R150Y/K170L/ | ++ |
| | Y392A | Q246K/A311G/K336S/K340W/R357E/A388K/Y392A/ | |
| | | R394K/V401R/Y404L/L514S/T609E/A629K/E659R/ | |
| | | D660W/W664P/E782V | |
| 813/814 | T56A/D137G/ | A32V/R38G/D137G/N138R/A143N/R150Y/K170L/ | + |
| | A143N/L404Y/ | Q246K/A311G/K336S/K340W/R357E/A388K/R394K/ | |
| | K720S | V401R/L514S/T609E/A629K/E659R/D660W/W664P/ | |
| | | K720S/E782V | |
| 815/816 | T56E/A143N/ | A32V/R38G/A56E/N138R/A143N/R150Y/K170L/ | ++ |
| | K246R | Q246R/A311G/K336S/K340W/R357E/A388K/R394K/ | |
| | | V401R/Y404L/L514S/T609E/A629K/E659R/D660W/ | |
| | | W664P/E782V | |
| 817/818 | T56E/Y392R | A32V/R38G/A56E/N138R/R150Y/K170L/Q246K/ | + |
| | | A311G/K336S/K340W/R357E/A388K/Y392R/R394K/ | |
| | | V401R/Y404L/L514S/T609E/A629K/E659R/D660W/ | |
| | | W664P/E782V | |
| 819/820 | Y392R/L404Y | A32V/R38G/A56T/N138R/R150Y/K170L/Q246K/ | + |
| | | A311G/K336S/K340W/R357E/A388K/Y392R/R394K/ | |
| | | V401R/L514S/T609E/A629K/E659R/D660W/ | |
| | | W664P/E782V | |
| 821/822 | Y392R | A32V/R38G/A56T/N138R/R150Y/K170L/Q246K/ | + |
| | | A311G/K336S/K340W/R357E/A388K/Y392R/R394K/ | |
| | | V401R/Y404L/L514S/T609E/A629K/E659R/D660W/ | |
| | | W664P/E782V | |
| 823/824 | D137G/Y392R | A32V/R38G/A56T/D137G/N138R/R150Y/K170L/Q246K/ | + |
| | | A311G/K336S/K340W/R357E/A388K/Y392R/R394K/ | |
| | | V401R/Y404L/L514S/T609E/A629K/E659R/D660W/ | |
| | | W664P/E782V | |
| 825/826 | T56E/D137G | A32V/R38G/A56E/D137G/N138R/R150Y/K170L/Q246K/ | + |
| | | A311G/K336S/K340W/R357E/A388K/R394K/V401R/ | |
| | | Y404L/L514S/T609E/A629K/E659R/D660W/W664P/ | |
| | | E782V | |
| 827/828 | T56E/D196S/ | A32V/R38G/A56E/N138R/R150Y/K170L/D196S/Q246K/ | + |
| | Y392A | A311G/K336S/K340W/R357E/A388K/Y392A/R394K/ | |
| | | V401R/Y404L/L514S/T609E/A629K/E659R/D660W/ | |
| | | W664P/E782V | |

Levels of increased activity were determined relative to the reference polypeptide of SEQ ID NO: 800 and defined as follows: “+” 1.13 to 1.44 (first 50%), “++” >1.44 (next 30%), “+++” >1.73 (top 20%)

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. An engineered RNA polymerase, or a functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 8 to 890 of SEQ ID NO: 2, 4, 32, 44, 156, 234, 238, 386, 394, 588, 604, 686, 692, 712, or 800, or to a reference sequence corresponding to SEQ ID NO: 2, 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 2, 4, 32, 44, 156, 234, 238, 386, 394, 588, 604, 686, 692, 712, or 800, or relative to the reference sequence corresponding to SEQ ID NO: 2, 4, 32, 44, 156, 234, 238, 386, 394, 588, 604, 686, 692, 712, or 800.

2. The engineered RNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 2, or to the reference sequence corresponding to SEQ ID NO: 2, wherein the amino acid sequence comprises one or more substitutions relative to the reference sequence corresponding to residues 8 to 890 of SEQ ID NO: 2, or relative to the reference sequence corresponding to SEQ ID NO: 2.

3. The engineered RNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 2, or relative to the reference sequence corresponding to SEQ ID NO: 2.

4. The engineered RNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 8 to 890 of an even numbered SEQ ID NO. of SEQ ID NOs: 4-828, or to a reference sequence corresponding to an even numbered SEQ ID NO. of SEQ ID NOs: 4-828, wherein the amino acid sequence comprises one or more substitutions relative to the reference sequence corresponding to residues 8 to 890 of SEQ ID NO: 2, or relative to the reference sequence corresponding to SEQ ID NO: 2.

5. The engineered RNA polymerase of claim 1, wherein the amino acid sequence of the engineered RNA polymerase comprises at least a substitution at amino acid position 13, 14, 15, 17, 19, 21, 32, 33, 38, 45, 52, 54, 55, 56, 56, 57, 60, 61, 62, 63, 67, 69, 72, 75, 78, 81, 82, 83, 84, 89, 93, 95, 100, 105, 111, 115, 122, 124, 127, 131, 133, 135, 137, 138, 139, 140, 141, 143, 150, 160, 161, 162, 164, 166, 168, 169, 170, 171, 172, 173, 179, 185, 187, 193, 196, 207, 212, 217, 218, 221, 227, 237, 239, 240, 245, 246, 250, 253, 254, 257, 261, 272, 274, 275, 311, 336, 340, 342, 351, 357, 367, 375, 379, 382, 388, 391, 392, 394, 397, 401, 402, 403, 404, 419, 424, 437, 440, 494, 510, 514, 517, 527, 529, 530, 531, 533, 536, 589, 594, 598, 607, 609, 615, 616, 617, 625, 629, 648, 650, 655, 659, 660, 663, 664, 666, 670, 672, 690, 693, 696, 718, 720, 721, 749, 759, 763, 773, 775, 779, 782, 782, 810, 854, 857, 867, 875, or 878, or combinations thereof, wherein the amino acid positions are relative to the reference sequence corresponding to residues 8 to 890 of SEQ ID NO: 2, or relative to the reference sequence corresponding to SEQ ID NO: 2.

6. The engineered RNA polymerase claim 5, wherein the amino acid sequence of the engineered RNA polymerase comprises at least a substitution or an amino acid residue 13N, 14Q/Y, 15T, 17N, 19E, 21G, 32S/T/V, 33E, 38G, 45K, 52T, 54H, 55D/S/W, 56E/G/N/T/V, 57G, 60G/L/M/R, 61W/Y, 62T, 63S, 67R, 69S, 72R, 75S/Y, 78S, 81C/L, 82S, 83K, 84A, 89Q, 93Y, 95Y, 100R, 105R, 111K, 115L/V, 122I, 124F, 127G, 131T, 133C, 135G/K, 137G, 138R, 139R, 140R, 141A, 143N/Q/R/V, 150Q/Y, 160F/L, 161R, 162V, 164W, 166D/R/T/V, 168K/L/R/S/T/V/W, 169G/L, 170L/Q/R/S, 171E/F/L/Y, 172A/E/G/K/R/V, 173D/H/L/Y, 179N, 185I/L/P, 187G/H/L, 193G, 196A/C/G/S/V, 207S, 212R/Y, 217P/V, 218Q/T, 221C, 227V, 237L, 239L/R, 240M, 245C, 246K/R/V, 250R, 253Y, 254G/S, 257L/V, 261M, 272N, 274G/L, 275A/Y, 311G, 336L/M/S/V, 340E/W, L342Q, 351L, 357E/P, 367F/P/T, 375F/P, 379A/S, 382G/L/R, 388K/R, 391F/S, 392A/M/R, 394K, 397S, 401R, 402T, 403V, 404I/L/S/Y, 419R/S, 424A, 437P, 440K, 494G, 510V, 514I/S, 517R, 527E, 529R, 530N, 531Q, 533S, 536E/V, 589C, 594G, 598A/K/L/R/V, 607Y, 609C/E/F/P/W, 615A, 616A, 617R, 625C, 629D/K/R, 648F, 650M, 655M/R, 659R, 660H/W/Y, 663L, 664K/P, 666M, 670Q/R, 672G/P/R/S/T, 690S, 693R, 696L, 718F/N/S/V/Y, 720A/G/I/P/S, 721A/E/M/S/T/Y, 749R, 759M, 763S, 773A, 775K/R, H779R, 782R/V, 810K, 854T, 857V, 867Q, 875C, or 878M, or combinations thereof, wherein the amino acid positions are relative to the reference sequence corresponding to residues 8 to 890 of SEQ ID NO: 2, or relative to the reference sequence corresponding to SEQ ID NO: 2.

7. The engineered RNA polymerase of claim 1, wherein the amino acid sequence of the engineered RNA polymerase comprises at least a substitution at amino acid position 32, 38, 56, 138, 150, 170, 171, 173, 246, 311, 336, 340, 357, 388, 394, 401, 404, 437, 514, 536, 609, 629, 659, 660, 664, 779, or 782, or combinations thereof, wherein the amino acid positions are relative to the reference sequence corresponding to residues 8 to 890 of SEQ ID NO: 2, or relative to the reference sequence corresponding to SEQ ID NO: 2.

8. (canceled)

9. (canceled)

10. (canceled)

11. (canceled)

12. (canceled)

13. The engineered RNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800.

14. (canceled)

15. The engineered RNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, 32, 44, 156, 234, 238, 386, 394, 588, 604, 686, 692, 712, or 800, or to the reference sequence corresponding to SEQ ID NO: 4, 32, 44, 156, 234, 238, 386, 394, 588, 604, 686, 692, 712, or 800, wherein the amino acid sequence comprises one or more substitutions relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

16. The engineered RNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of an even-numbered SEQ ID NO. of SEQ ID NOs: 4-828, or to the reference sequence corresponding to an even-numbered SEQ ID NO. of SEQ ID NOs: 4-828, wherein the amino acid sequence comprises one or more substitutions relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

17. The engineered RNA polymerase of claim 16, wherein the amino acid sequence of the engineered RNA polymerase comprises at least a substitution at amino acid position 13, 14, 15, 17, 19, 21, 32, 33, 38, 45, 52, 54, 55, 56, 56, 57, 60, 61, 62, 63, 67, 69, 72, 75, 78, 81, 82, 83, 84, 89, 93, 95, 100, 105, 111, 115, 122, 124, 127, 131, 133, 135, 137, 138, 139, 140, 141, 143, 150, 160, 161, 162, 164, 166, 168, 169, 170, 171, 172, 173, 179, 185, 187, 193, 196, 207, 212, 217, 218, 221, 227, 237, 239, 240, 245, 246, 250, 253, 254, 257, 261, 272, 274, 275, 311, 336, 340, 342, 351, 357, 367, 375, 379, 382, 388, 391, 392, 394, 397, 401, 402, 403, 404, 419, 424, 437, 440, 494, 510, 514, 517, 527, 529, 530, 531, 533, 536, 589, 594, 598, 607, 609, 615, 616, 617, 625, 629, 648, 650, 655, 659, 660, 663, 664, 666, 670, 672, 690, 693, 696, 718, 720, 721, 749, 759, 763, 773, 775, 779, 782, 782, 810, 854, 857, 867, 875, or 878 or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

18. The engineered RNA polymerase of claim 15, wherein the amino acid sequence of the engineered RNA polymerase comprises at least a substitution or an amino acid residue 13N, 14Q/Y, 15T, 17N, 19E, 21G, 32S/TV, 33E, 38G/R, 45K, 52T, 54H, 55D/S/W, 56A/E/G/N/T/V, 57G, 60G/L/M/R, 61W/Y, 62T, 63S, 67R, 69S, 72R, 75S/Y, 78S, 81C/L, 82S, 83K, 84A, 89Q, 93Y, 95Y, 100R, 105R, 111K, 115L/V, 122I, 124F, 127G, 131T, 133C, 135G/K, 137G, 138N/R, 139R, 140R, 141A, 143N/Q/R/V, 150Q/Y, 160F/L, 161R, 162V, 164W, 166D/R/T/V, 168K/L/R/S/T/V/W, 169G/L, 170L/Q/R/S, 171E/F/L/Y, 172A/E/G/K/R/V, 173D/H/L/Y/V, 179N, 185I/L/P, 187G/H/L, 193G, 196A/C/G/S/V, 207S, 212R/Y, 217P/V, 218Q/T, 221C, 227V, 237L, 239L/R, 240M, 245C, 246K/RN, 250R, 253Y, 254G/S, 257L/V, 261M, 272N, 274G/L, 275A/Y, 311A/G, 336K/L/M/S/V, 340E/K/W, 342Q, 351L, 357E/P, 367F/P/T, 375F/P, 379A/S, 382G/L/R, 388A/K/R, 391F/S, 392A/M/R, 394K, 397S, 401R, 402T, 403V, 404I/L/S/Y, 419R/S, 424A, 437P/S, 440K, 494G, 510V, 514I/L/S, 517R, 527E, 529R, 530N, 531Q, 533S, 536E/V, 589C, 594G, 598A/K/L/R/V, 607Y, 609C/E/F/P/W, 615A, 616A, 617R, 625C, 629A/D/K/R, 648F, 650M, 655M/R, 659E/R, 660H/W/Y, 663L, 664K/P, 666M, 670Q/R, 672G/P/R/S/T, 690S, 693R, 696L, 718F/N/S/V/Y, 720A/G/I/P/S, 721A/E/M/S/T/Y, 749R, 759M, 763S, 773A, 775K/R, 779R, 782E/R, 782V, 810K, 854T, 857V, 867Q, 875C, or 878M, or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO:
4, 32, 44, 156, 234, 238, 386, 394, 588, 604, 686, 692, 712, or 800.

19. The engineered RNA polymerase of claim 15, wherein the amino acid sequence of the engineered RNA polymerase comprises at least a substitution at amino acid position 32, 38, 56, 138, 150, 170, 171, 173, 246, 311, 336, 340, 357, 388, 394, 401, 404, 437, 514, 536, 609, 629, 659, 660, 664, 779, or 782, or combinations thereof, wherein the amino acid positions are relative to the reference sequence corresponding to residues 8 to 890 of SEQ ID NO: 4, 32, 44, 156, 234, 238, 386, 394, 588, 604, 686, 692, 712, or 800, or relative to the reference sequence corresponding to SEQ ID NO: 4, 32, 44, 156, 234, 238, 386, 394, 588, 604, 686, 692, 712, or 800.

20. (canceled)

21. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 4, or to the reference sequence corresponding to SEQ ID NO: 4, wherein the amino acid sequence comprises one or more substitutions relative to the reference sequence corresponding to residues 8 to 890 of SEQ ID NO: 4, or relative to the reference sequence corresponding to SEQ ID NO: 4.

22. The engineered RNA polymerase of claim 21, wherein the amino acid sequence of the engineered RNA polymerase comprises at least a substitution or substitution set at amino acid position(s) 311/388/404/440, 138/340, 138/311/336/340/388, 311/388/440, 138/311/340/388/404/440, 138/336/342, or 351/388, wherein the amino acid positions are relative to the reference sequence corresponding to residues 8 to 890 of SEQ ID NO: 4, or relative to the reference sequence corresponding to SEQ ID NO: 4.

23. (canceled)

24. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 32, or to the reference sequence corresponding to SEQ ID NO: 32, wherein the amino acid sequence comprises one or more substitutions relative to the reference sequence corresponding to residues 8 to 890 of SEQ ID NO: 32, or relative to the reference sequence corresponding to SEQ ID NO: 32.

25. The engineered RNA polymerase of claim 24, wherein the amino acid sequence of the engineered RNA polymerase comprises at least a substitution or substitution set at amino acid position(s) 514, 38/514, 38/394/514, 38/394/514/664, 394/514/875, 394/514, 38/124/394/514, 171/878, 170, 173, 172, 170/629, 171, 168, 166, 166/272/274/275, 169, 207, 168/217/218, 171/530, 382, 773, 670, 379, 392, 419, 57, 164, 810, 659, 54, 172/173/514/659/660, 170/171/172/514/659, 173/514/659/660, 173/514/659/810, 170/340/514/659/660/810, 170/171/173/336/340/514/659/660, 57/171/173/514/659/660/810, 170/173/514/659/660/810, 57/171/172/336/340/514/659, 173/514, 171/172/173/514/659/660, 57/170/171/173/514/659/660/810, 57/173/514, 172/173/514/857, 57/173/340/514, 170/171/173/336/514, 171/514, 57/170/173/336/514, 170/336, 173/514/659, 57/514, or 173/810, wherein the amino acid positions are relative to the reference sequence corresponding to residues 8 to 890 of SEQ ID NO: 32, or relative to the reference sequence corresponding to SEQ ID NO: 32.

26. (canceled)

27. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 44, or to the reference sequence corresponding to SEQ ID NO: 44, wherein the amino acid sequence comprises one or more substitutions relative to the reference sequence corresponding to residues 8 to 890 of SEQ ID NO: 44, or relative to the reference sequence corresponding to SEQ ID NO: 44.

28. The engineered RNA polymerase of claim 27, wherein the amino acid sequence of the engineered RNA polymerase comprises at least a substitution or substitution set at amino acid position(s) 336, 143/336/340/782, or 143/336/340/357/514, wherein the amino acid positions are relative to the reference sequence corresponding to residues 8 to 890 of SEQ ID NO: 44, or relative to the reference sequence corresponding to SEQ ID NO: 44.

29. (canceled)

30. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 156, or to the reference sequence corresponding to SEQ ID NO: 156, wherein the amino acid sequence comprises one or more substitutions relative to the reference sequence corresponding to residues 8 to 890 of SEQ ID NO: 156, or relative to the reference sequence corresponding to SEQ ID NO: 156.

31. The engineered RNA polymerase of claim 30, wherein the amino acid sequence of the engineered RNA polymerase comprises at least a substitution or substitution set at amino acid position(s) 207, 419, 207/336/340, 166/169, 207/670, 166/336/340, 207/419/670, 207/336/340/382/419/670, 207/382, 670, 336/670, 340/670, 382, 127/141/207/336/382/670, 207/336/382/419/670, 419/670, 166/168/207/336/340/419/670, 166/340, or 207/336/340/670, wherein the amino acid positions are relative to the reference sequence corresponding to residues 8 to 890 of SEQ ID NO: 156, or relative to the reference sequence corresponding to SEQ ID NO: 156.

32. (canceled)

33. The engineered RNA polymerase of claim 30, wherein the amino acid sequence of the engineered RNA polymerase comprises at least a substitution or substitution set at amino acid position(s) 38/437/779, 38/437, 38, 38/207/437/779, 517/779, 38/437/527/664, 38/207/379/437, 664/670/779, 38/779, 38/437/517, 437/779, 38/437/607, 38/379/779, or 517/670/779, wherein the amino acid positions are relative to the reference sequence corresponding to SEQ ID NO: 156.

34. (canceled)

35. The engineered RNA polymerase of claim 30, wherein the amino acid sequence of the engineered RNA polymerase comprises at least a substitution set at amino acid positions 357/394/664 or 138/311/336/340/357/388/394/404/664, wherein the amino acid positions are relative to the reference sequence corresponding to residues 8 to 890 of SEQ ID NO: 156, or relative to the reference sequence corresponding to SEQ ID NO: 156.

36. (canceled)

37. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 234, or to the reference sequence corresponding to SEQ ID NO: 234, wherein the amino acid sequence comprises one or more substitutions relative to the reference sequence corresponding to residues 8 to 890 of SEQ ID NO: 234, or relative to the reference sequence corresponding to SEQ ID NO: 234.

38. The engineered RNA polymerase of claim 37, wherein the amino acid sequence of the engineered RNA polymerase comprises at least a substitution or substitution set at amino acid position(s) 720, 196, 609, 246, 185, 594, 212, 494, 375, 721, 72, 867, 160, 718, 187, 170/173, 615, 137/138, 854, 131, 38/185/187/437, 185/187, 437/609, 38/609, 38/185/187/437/609, 437/609/720, 185/187/609, 185/187/437, 437/721, 609/720, 185/187/246, or 185/187/437/609/720, wherein the amino acid positions are relative to the reference sequence corresponding to residues 8 to 890 of SEQ ID NO: 234, or relative to the reference sequence corresponding to SEQ ID NO: 234.

39. (canceled)

40. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 386, or to the reference sequence corresponding to SEQ ID NO: 386, wherein the amino acid sequence comprises one or more substitutions relative to the reference sequence corresponding to residues 8 to 890 of SEQ ID NO: 386, or relative to the reference sequence corresponding to SEQ ID NO: 386.

41. The engineered RNA polymerase of claim 40, wherein the amino acid sequence of the engineered RNA polymerase comprises at least a substitution or substitution set at amino acid position(s) 170/609, 38/185/187/609, 38/185/187, 38/170/173, 38/173/185/187/720, 170/185/187/609, 609, 170/185/187/609/721, 38/609, 38/170/246/609, 185/187/609/718, 38/185/187/609/718/720, 38/170/246/609/718, 720, 246/609, 38/185/187/246/720, 38/609/721, 38/721, 38/720/721, 38/170/609/718/720, 38/718, 38/185/187/609/718/721, 529/609, 38/609/718, 185/187/609/718/720, 185/187/609/718/721, 38/170/718/720/721, 170/185/187/718/720, 38/173/185/187/246/609/720, 609/718/720, 170/185/187/718/720/721, 38/170/173/246/609/718, 246/721, 38/718/720, or 38/609/718/720, wherein the amino acid positions are relative to the reference sequence corresponding to residues 8 to 890 of SEQ ID NO: 386, or relative to the reference sequence corresponding to SEQ ID NO: 386.

42. (canceled)

43. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 238, or to the reference sequence corresponding to SEQ ID NO: 238, wherein the amino acid sequence comprises one or more substitutions relative to the reference sequence corresponding to residues 8 to 890 of SEQ ID NO: 238, or relative to the reference sequence corresponding to SEQ ID NO: 238.

44. The engineered RNA polymerase of claim 43, wherein the amino acid sequence of the engineered RNA polymerase comprises at least a substitution or substitution set at amino acid position(s) 143, 105, 775/779, 135, 100, 246, 779/782, 749, 237, 162, 83, 218, 227, 115, 274, 81, 122, 61, 32, 139, 75, 239, 257, 240, 69/161, 253, 14, 221, 84, 133/150, 60, 275, 89, 15, 254, 625, 14/67, 21, 93, 250, 217, 19, 95, 759, 62, or 140, wherein the amino acid positions are relative to the reference sequence corresponding to residues 8 to 890 of SEQ ID NO: 238, or relative to the reference sequence corresponding to SEQ ID NO: 238.

45. (canceled)

46. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 394, or to the reference sequence corresponding to SEQ ID NO: 394, wherein the amino acid sequence comprises one or more substitutions relative to the reference sequence corresponding to residues 8 to 890 of SEQ ID NO: 394, or relative to the reference sequence corresponding to SEQ ID NO: 394.

47. The engineered RNA polymerase of claim 46, wherein the amino acid sequence of the engineered RNA polymerase comprises at least a substitution or substitution set at amino acid position(s) 609/779, 246/609, 246, 609/775/779, 105, 143/609/775, 105/135/609/775, or 111/609, 609, wherein the amino acid positions are relative to the reference sequence corresponding to residues 8 to 890 of SEQ ID NO: 394, or relative to the reference sequence corresponding to SEQ ID NO: 394.

48. (canceled)

49. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 588, or to the reference sequence corresponding to SEQ ID NO: 588, wherein the amino acid sequence comprises one or more substitutions relative to the reference sequence corresponding to residues 8 to 890 of SEQ ID NO: 588, or relative to the reference sequence corresponding to SEQ ID NO: 588.

50. The engineered RNA polymerase of claim 49, wherein the amino acid sequence of the engineered RNA polymerase comprises at least a substitution or substitution set at amino acid position(s) 32, 531/533, 115, 83, 32/60/61, 55, 589, 357, 617, 172, 598, 56/193, 56, 536, 52, 17, 261, 78, 629, 63, 82, 207, 666, 150, 45, 135, 13, 33, or 245, wherein the amino acid positions are relative to the reference sequence corresponding to residues 8 to 890 of SEQ ID NO: 588, or relative to the reference sequence corresponding to SEQ ID NO: 588.

51. (canceled)

52. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 604, or to the reference sequence corresponding to SEQ ID NO: 604, wherein the amino acid sequence comprises one or more substitutions relative to the reference sequence corresponding to residues 8 to 890 of SEQ ID NO: 604, or relative to the reference sequence corresponding to SEQ ID NO: 604.

53. The engineered RNA polymerase of claim 52, wherein the amino acid sequence of the engineered RNA polymerase comprises at least a substitution or substitution set at amino acid position(s) 207/245/629, 17/56/150/598, 56/629, 536, 185, 56/150, 17/56/150/207/245/598/617/629, 56/150/536/598, 56/536, 185/666, 150, or 143/150/246/782, wherein the amino acid positions are relative to the reference sequence corresponding to residues 8 to 890 of SEQ ID NO: 604, or relative to the reference sequence corresponding to SEQ ID NO: 604.

54. (canceled)

55. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 686, or to the reference sequence corresponding to SEQ ID NO: 686, wherein the amino acid sequence comprises one or more substitutions relative to the reference sequence corresponding to residues 8 to 890 of SEQ ID NO: 686, or relative to the reference sequence corresponding to SEQ ID NO: 686.

56. The engineered RNA polymerase of claim 55, wherein the amino acid sequence of the engineered RNA polymerase comprises at least a substitution or substitution set at amino acid position(s) 143, 246/424/648/782, 246/659/782, 56/150/629/660/782, 721/782, 782, 196/659/782, or 720/721/782, wherein the amino acid positions are relative to the reference sequence corresponding to residues 8 to 890 of SEQ ID NO: 686, or relative to the reference sequence corresponding to SEQ ID NO: 686.

57. (canceled)

58. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 692, or to the reference sequence corresponding to SEQ ID NO: 692, wherein the amino acid sequence comprises one or more substitutions relative to the reference sequence corresponding to residues 8 to 890 of SEQ ID NO: 692, or relative to the reference sequence corresponding to SEQ ID NO: 692.

59. The engineered RNA polymerase of claim 58, wherein the amino acid sequence of the engineered RNA polymerase comprises at least a substitution or substitution set at amino acid position(s) 143/659/782, 166/171/246/311, 143/721, 782, or 143/171/659/782, wherein the amino acid positions are relative to the reference sequence corresponding to residues 8 to 890 of SEQ ID NO: 692, or relative to the reference sequence corresponding to SEQ ID NO: 692.

60. (canceled)

61. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 712, or to the reference sequence corresponding to SEQ ID NO: 712, wherein the amino acid sequence comprises one or more substitutions relative to the reference sequence corresponding to residues 8 to 890 of SEQ ID NO: 712, or relative to the reference sequence corresponding to SEQ ID NO: 712.

62. The engineered RNA polymerase of claim 61, wherein the amino acid sequence of the engineered RNA polymerase comprises at least a substitution or substitution set at amino acid position(s) 367, 382, 670, 672, 357, 388, 693, 397, 375, 137/382/782, 655, 137/382/392, 391, 137/655/721/763, 696, 663, 137/382, 137/382/392/650/655/782, 137/382/629/660/672, 616, 690, 179, 137, 357/382/670, 388/670/672, 357/375, 357/672, 629, or 655/670, wherein the amino acid positions are relative to the reference sequence corresponding to residues 8 to 890 of SEQ ID NO: 712, or relative to the reference sequence corresponding to SEQ ID NO: 712.

63. (canceled)

64. The engineered RNA polymerase of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 8 to 890 of SEQ ID NO: 800, or to the reference sequence corresponding to SEQ ID NO: 800, wherein the amino acid sequence comprises one or more substitutions relative to the reference sequence corresponding to residues 8 to 890 of SEQ ID NO: 800, or relative to the reference sequence corresponding to SEQ ID NO: 800.

65. The engineered RNA polymerase of claim 64, wherein the amino acid sequence of the engineered RNA polymerase comprises at least a substitution or substitution set at amino acid position(s) 137, 56/311/382, 56/137/143/721, 137/392, 56/137/392, 56/137/143/404/720, 56/143/246, 56/392, 392/404, 392, 56/137, or 56/196/392, wherein the amino acid positions are relative to the reference sequence corresponding to residues 8 to 890 of SEQ ID NO: 800, or relative to the reference sequence corresponding to SEQ ID NO: 800.

66. (canceled)

67. (canceled)

68. (canceled)

69. (canceled)

70. The engineered RNA polymerase of claim 1, wherein the amino acid sequence of the engineered RNA polymerase comprises residues 8 to 890 of an even numbered SEQ ID NO. of SEQ ID NOs: 4-828, or comprises an even numbered SEQ ID NO. of SEQ ID NOs: 4-828, optionally wherein the amino acid sequence has 1, 2, 3, 4, 5, 6, 7, 8, 9, or up to 10 substitutions.

71. (canceled)

72. (canceled)

73. The engineered RNA polymerase of claim 1, wherein the engineered RNA polymerase exhibits an improved property compared to a reference RNA polymerase, wherein the improved property is-selected from i) increased activity, ii) increased thermostability, iii) increased processivity, and iv) increased RNA product integrity, or any combination of i), ii), iii) and iv), and wherein the reference RNA polymerase has the sequence corresponding to residues 8 to 890 of SEQ ID NO: 2, 4, 32, 44, 156, 234, 238, 386, 394, 588, 604, 686, 692, 712, or 800, or the sequence corresponding to SEQ ID NO: 2, 4, 32, 44, 156, 234, 238, 386, 394, 588, 604, 686, 692, 712, or 800.

74. (canceled)

75. (canceled)

76. A recombinant polynucleotide encoding an engineered RNA polymerase of claim 1.

77. (canceled)

78. (canceled)

79. (canceled)

80. (canceled)

81. (canceled)

82. An expression vector comprising at least one recombinant polynucleotide of claim 76.

83. (canceled)

84. (canceled)

85. A host cell comprising an expression vector of claim 82.

86. (canceled)

87. A method of producing an engineered RNA polymerase polypeptide in a host cell comprising culturing a host cell of claim 85, under suitable culture conditions, such that at least one engineered RNA polymerase is produced.

88. (canceled)

89. (canceled)

90. (canceled)

91. (canceled)

92. (canceled)

93. (canceled)

94. (canceled)

95. (canceled)

96. A method of producing RNA in vitro, comprising contacting a target DNA template with an engineered RNA polymerase of claim 1 in presence of one or more nucleotide triphosphates under conditions suitable for transcription of the DNA template.

97. (canceled)

98. (canceled)

99. (canceled)

100. (canceled)

101. A kit comprising an engineered RNA polymerase of claim 1.

102. (canceled)
